(19) United States
(12) Patent Application Publication
(10) Pub. No.: US 2026/0234240 A1
(43) Pub. Date: Aug. 13, 2026

(54) IL-10 RECEPTOR ANTIBODIES AND METHODS OF USE THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US); Tri-Institutional Therapeutics Discovery Institute, Inc., New York, NY (US)

(72) Inventors: Jedd D WOLCHOK, New York, NY (US); Taha MERGHOUB, New York, NY (US); Danny Nejad KHALIL, New York, NY (US); Elizabeth NYAKATURA, New York, NY (US)

(21) Appl. No.: 19/472,835

(22) PCT Filed: Apr. 8, 2024
(86) PCT No.: PCT/US2024/023601

§ 371 Date: Oct. 6, 2025

Related U.S. Application Data
(60) Provisional application No. 63/457,651, filed on Apr. 6, 2023.

(51) Int. Cl. C07K 16/24; A61K 38/00; A61K 45/06; A61P 35/04; C07K 16/28; C12N 15/63; G01N 33/543; G01N 33/575; G01N 33/68

(52) U.S. Cl. CPC C07K 16/244; A61K 38/00; A61K 45/06; A61P 35/04; C07K 16/2866; C12N 15/63; G01N 33/54386; G01N 33/5758; G01N 33/6893; C07K 2317/21; C07K 2317/24; C07K 2317/31; C07K 2317/55; C07K 2317/565; C07K 2317/622; C07K 2317/76; C07K 2317/92; G01N 2333/5428; G01N 2474/10; G01N 2474/20

(57) ABSTRACT

The invention is directed to human monoclonal antibodies that bind to Interleukin 10 (IL-10) and methods of use thereof.

This application claims priority to U.S. Provisional Application No. 63/457,651, filed on Apr. 6, 2023, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The application contains a Sequence Listing which has been submitted electronically in ASCII format and is hereby incorporated by reference in its entirety. Said ASCII copy, created on [ ], is named [ ] and is [ ] bytes in size.

### FIELD OF THE INVENTION

This invention is directed to antibodies against IL-10 (also known as Interleukin 10), and methods of use thereof.

### BACKGROUND OF THE INVENTION

Cancers refractory to immune checkpoint (PD-1, PD-L1, and CTLA-4) blockade fail to mount significant anti-tumor T lymphocyte responses.

### SUMMARY OF THE INVENTION

Aspects of the invention are directed to isolated monoclonal antibodies or fragments thereof that specifically bind to Interleukin 10 (IL-10) or IL-10 receptor. In embodiments, the monoclonal antibody comprises a heavy chain, light chain or a combination thereof.

In embodiments, the antibody is an IL-10 blocking antibody or an IL-10 receptor blocking antibody.

In embodiments, IL-10 is human IL-10.

In embodiments, the antibody is fully human or humanized.

In embodiments, the antibody is monospecific, bispecific, or multispecific.

In embodiments, the antibody is an IgG.

In embodiments, the antibody is selected from the group consisting of 49G08_HIL2, 49G08, 48C11, 49H07, 48C06, 53D07, 53H08, 42B02, 43C05, 43F05, 15A02, and 21B08.

In embodiments, the antibody competes with the binding of 49G08_HIL2, 49G08, 48C11, 49H07, 48C06, 53D07, 53H08, 42B02, 43C05, 43F05, 15A02, or 21B08.

In embodiments, the antibody comprises a heavy chain variable region (V_{H}) comprising an amino acid sequence as indicated below, or a sequence at least 90% identical thereto:

a.
EVQLVESGGGLVQPGGSLRLSCAASGFTFSDYYMYWVRQAPGKGLE
WVSTISDGGSYTYYPDSVKGRFTISRDNAKNNLYLQMNSLRAEDTAV
YYCARGYYYGLFTYWGQGTLVTVSS;
b.
EVQLVESGGGLVKPGGSLKLSCAASGFTFSDYYMYWVRQTPEKRLE
WVATISDGGSYTYYPDSVKGRFTISRDNAKNNLYLQMSSLKSEDTAM
FYCARGYYYGLFTYWGQGTLVTVSA;
c.
EVQLQQSGPDLVKPGASVRMSCKASGYTFTNYVMHWVKQRPGQGLE
WIGYISPYNDGTKYNEKFKGKATLTSDKSSSTAYMELNSLTSEDSAVY
YCAGGDYSYAMDYWGQGTSVTVSS;
d.
EVQLQQSGPDLVKPGASVKMSCKASGYTFTHYVMHWVKQKPGQGL
EWIGYISPYNDGIKYNEKFKGKATLTSDKSSSTAYMELSSLTSEDSAVY
YCAGGDYSYAMDYWGQGTSVTVSS;
e.
EVQLVESGGGLVKPGGSLKLSCAASGFTFSDYYIYWVRQTPEKRLEW
VATISDGGSYTYYPDSVKGRFTISRDNAKNNLYLQMSSLKSEDTAMY
YCARGYYYGLFTYWGQGTLVTVSA;
f.
QVQLKQSGPSLVQPSQSLSITCTVSGFSLTTYGVHWVRQSPGKGLEWL
GVIWRGGSTDYNAGFMSRLSITKDNSKSQVFFKMNSLQADDTAIYYC
AKNSYYYGGNYYFDYWGQGTTLTVSS;
g.
QVQLKQSGPSLVQPSQSLSINCTVSGFSLTTYGVHWLRQSPGKGLEWL
GVIWRGGSTDYNAAFMSRLSITKDNSKSQVFFKMNSLQADDTAIYYC
AKPYYVYGAMDYWGQGTSVTVSS:
h.
QVQLQESGPGLVKPSETLSLTCAVSGGSLSSDYWTWIRQPAGKGLEWI
GRIFATGTTNYNPSLKSRVTMSIDTSKNQFSLKLNSVTAADTAVYYCA
RDDWYFDLWGRGTLVTVSS;
i.
QVQLQESGPGLVKPSETLSLTCTVSGGSISSDYWSWIRQPAGKRLEWI
GRIFTSGSTNYNPSLKSRVTMSVDTSKNQFSLKLNSLTAADTAVYYCA
RDDWYFDLWGRGTLVTVSS;
j.
QVQLQESGPGLVKPSETLSLTCTVSGGSISRDYWSWVRQPAGKGLEWI
GRIFTNGVTNYNPSLKSRVTMSVDTSKTQFSLKLSSVTAADTAVYFCA
RDDWYLDLWGRGTLVTVSS;
k.
QVQMKQSGPGLVQPSQSLSITCTVSGFSLTTYGVHWVRQSPGKGLEW
LGVIWSGGSTYYNAAFISRLSISKDNSKSQVIFKMNSLQVDDTAIYYCA
RWDGYSFYYGMDYWGQGTSVTVSS;
or
l.
QVQLKQSGPGLVQPSQSLSITCTVSGFSLTSYGVHWVRQSPGKGLEWL
GVIWSGGSTDYNAAFISRLSISKDNSKSQVFFKMNSLQADDTAIYYCA
RWDGDSFYYAMDYWGQGTSVTVSS.

In embodiments, the antibody comprises a light chain variable region (V_{L}) comprising an amino acid sequence as indicated below, or a sequence of at least 90% identical thereto:

a.
DIQMTQSPSSLSASVGDRVTITCRASQDVNTGLNWYQQKPGQAPK
LLIYWASSRQSGVPSRFSGSGSGTDFTLTISSLQPEDFATYYCQQ
HYSTPYTFGQGTKLEIK;
b.
DIVMTQSHKFMSTSVGDRVSITCKASQDVNTGVAWYQQKPGQSPK
LLIYWASTRHTGVPDRFTGSGSGTDYILTISSVQAEDLALYYCQQ
HYSTPYTFGGGTKLEIK;
c.
DIQMTQSPASLSVSVGETVTITCRVSENIYSNLAWYHQKQGKSPQ
LLVYAATNLADGVPSRFSGSKSGTQYSLKISSLQSEDFGSYYCQH
FWGTLTFGAGTKLELK;
d.
DIQMTQSPASLSVSVGETVTITCRVSENIYSNLAWYQQKQGKSPQ
LLVYAATNLADGVSSRFSGSVSGTQYSLKINSLQSEDFGSYYCQH
FWGTLTFGAGTKLELK;
e.
DIVMTQSHKFMSTSVGDRVSITCKASQDVSTGVAWYRQKPGRSPK
LLIYWASTRHTGVPDRFTGSGSGTDYILTISSVQAEDLALYYCQQ
HYSTPYTFGGGTKLEIK;
f.
DVVMTQTPFSLPVSLGDQASISCRSSQSLVHSNGNTHLHWYLQKP
GQSPKLLIYKVSNRFSGVPDRFSGSGSGTDFTLKISRVEAEDLGV
YFCSQSTHVPPTFGGGTKLEIK;
g.
DVLMTQTPLSLPVSLGDQASISCRSSESIVHSNGNTYLEWFLQKP
GQSPKLLIYKVSNRFSGVPDRFSGSGSGTDFTLKISRVEAEDLGV
YYCFQGSHVPLTFGAGTKLEIK;
h.
EIVMTQSPATLSVSPGERATLSCRASQSVSNNLAWYQERPGQAPR
LLIYGASTRTTGIPARFSGSGSGTEFILTISSLQPEDFAIYYCQQ
YNEWPWTFGQGTKVEIK;
i.
EIVMTQSPATLSVSPGERATLSCRASQSVSNNLAWYQQKPGQAPR
FLIYGASTRATGMPARFSGSGSGTEFTLTISSLQSEDFAVYFCQQ
YNEWPWTFGQGTKVEIK;
j.
EIVMTQSPATLSVSPGERATLSCRASQSVSNNLAWYQQTPGQAPR
FLIYGASTGATGIPARFSGSGSGTEFTLTITSLQSEDFAVYFCQQ
YNDWPWTFGQGTKVEIK;
k.
QAVVTQESALTTSPGETVTLTCRSSTGAVTTSNYANWVQEKPDHL
FTGLIGSTNNRAPGVPARFSGSLIGDKAALIITGAQTEDEARYFC
ALWYSNHLVFGGGTKLTVL;
or
1.
QAVVIQESALTTSPGETVTLTCRSSTGAVTTSNYASWVQEKPDHL
FIGLIGGTNNRAPGVPARFSGSLIGDKAALTITGAQTEDEAIYFC
ALWYSNHLVFGGGTKLTVL.

In embodiments, the monoclonal antibody comprises:
a. heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
b. a heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
c. a heavy chain with three CDRs comprising the amino acid sequences GYTFTNYV, ISPYNDGT, and AGGDYSYAMDY respectively, and/or a light chain with three CDRs comprising the amino acid sequences ENIYSN, AAT, and QHFWGTLT respectively;
d. a heavy chain with three CDRs comprising the amino acid sequences GYTFTHYV, ISPYNDGI, and AGGDYSYAMDY respectively, and/or a light chain with three CDRs comprising the amino acid sequences ENIYSN, AAT, and QHFWGTLT respectively;
e. a heavy chain with three CDRs comprising the amino acid sequences GFTFSDYY, ISDGGSYT, and ARGYYYGLFTY respectively, and/or a light chain with three CDRs comprising the amino acid sequences QDVSTG, WAS, and QQHYSTPYT respectively;
f. a heavy chain with three CDRs comprising the amino acid sequences GFSLTTYG, IWRGGST, and AKNSYYYGGNYYFDY respectively, and/or a light chain with three CDRs comprising the amino acid sequences QSLVHSNGNTH, KVS, and SQSTHVPPT respectively;
g. a heavy chain with three CDRs comprising the amino acid sequences GFSLTTYG, IWRGGST, and AKPYYVYGAMDY respectively, and/or a light chain with three CDRs comprising the amino acid sequences ESIVHSNGNTY, KVS, and FQGSHVPLT respectively;
h. a heavy chain with three CDRs comprising the amino acid sequences GGSLSSDY, IFATGTT, and ARDDWYFDL respectively, and/or a light chain with three CDRs comprising the amino acid sequences QSVSNN, GAS, and QQYNEWPWT respectively;
i. a heavy chain with three CDRs comprising the amino acid sequences GGSISSDY, IFTSGST, and ARDDWYFDL respectively, and/or a light chain with three CDRs comprising the amino acid sequences QSVSNN, GAS, and QQYNEWPWT respectively;
j. a heavy chain with three CDRs comprising the amino acid sequences GGSISRDY, IFTNGVT, and ARDDWYLDL respectively, and/or a light chain with three CDRs comprising the amino acid sequences QSVSNN, GAS, and QQYNDWPWT respectively;
k. a heavy chain with three CDRs comprising the amino acid sequences GFSLTTYG, IWSGGST, and ARWDGYSFYYGMDY respectively, and/or a light chain with three CDRs comprising the amino acid sequences TGAVTTSNY, STN, and ALWYSNHLV respectively; or
l. a heavy chain with three CDRs comprising the amino acid sequences GFSLTSYG, IWSGGST, and ARWDGDSFYYAMDY respectively, and/or a light chain with three CDRs comprising the amino acid sequences TGAVTTSNY, GTN, and ALWYSNHLV respectively.

In embodiments, the monoclonal antibody comprises:
a. a V_{H} according to the amino acid sequence of EVOLVESGGGLVQPGGSLRLSCAASGFTFSDYYMYWVRQAPGKG LEWVSTISDGGSYTYYPDSVKGRFTISRDNAKNNLYLQMNSLRAE DTAVYYCARGYYYGLFTYWGQGTLVTVSS or a sequence at least 90% identical thereto, and a V_{L} according to the amino acid sequence of DIQMTQSPSSLSASVGDRVTITCRASQDVNTGLNWYQQKPGQAPK LLIYWASSRQSGVPSRFSGSGSGTDFTLTISSLQPEDFATYYCQQHY STPYTFGQGTKLEIK or a sequence at least 90% identical thereto;
b. a V_{H} according to the amino acid sequence of EVQLVESGGGLVKPGGSLKLSCAASGFTFSDYYMYWVRQTPEKR LEWVATISDGGSYTYYPDSVKGRFTISRDNAKNNLYLQMSSLKSE DTAMFYCARGYYYGLFTYWGQGTLVTVSA or a sequence at least 90% identical thereto, and a V_{L} according to the amino acid sequence of DIVMTQSHKFMSTSVGDRVSITCKASQDVNTGVAWYQQKPGQSP KLLIYWASTRHTGVPDRFTGSGSGTDYILTISSVQAEDLALYYCQQ HYSTPYTFGGGTKLEIK or a sequence at least 90% identical thereto;
c. a V_{H} according to the amino acid sequence of EVQLQQSGPDLVKPGASVRMSCKASGYTFTNYVMHWVKQRPGQ GLEWIGYISPYNDGTKYNEKFKGKATLTSDKSSSTAYMELNSLTSE DSAVYYCAGGDYSYAMDYWGQGTSVTVSS or a sequence at least 90% identical thereto, and a V_{L} according to the amino acid sequence of DIQMTQSPASLSVSVGETVTITCRVSENIYSNLAWYHQKQGKSPQL LVYAATNLADGVPSRFSGSKSGTQYSLKISSLQSEDFGSYYCQHFW GTLTFGAGTKLELK or a sequence at least 90% identical thereto;
d. a V_{H} according to the amino acid sequence of EVQLQQSGPDLVKPGASVKMSCKASGYTFTHYVMHWVKQKPGQ GLEWIGYISPYNDGIKYNEKFKGKATLTSDKSSSTAYMELSSLTSE DSAVYYCAGGDYSYAMDYWGQGTSVTVSS or a sequence at least 90% identical thereto, and a V_{L} according to the amino acid sequence of DIQMTQSPASLSVSVGETVTITCRVSENIYSNLAWYQQKQGKSPQL LVYAATNLADGVSSRFSGSVSGTQYSLKINSLQSEDFGSYYCQHF WGTLTFGAGTKLELK or a sequence at least 90% identical thereto;
e. a V_{H} according to the amino acid sequence of EVOLVESGGGLVKPGGSLKLSCAASGFTFSDYYIYWVRQTPEKRL EWVATISDGGSYTYYPDSVKGRFTISRDNAKNNLYLQMSSLKSED TAMYYCARGYYYGLFTYWGQGTLVTVSA or a sequence at least 90% identical thereto, and a V_{L} according to the amino acid sequence of DIVMTQSHKFMSTSVGDRVSITCKASQDVSTGVAWYROKPGRSPK LLIYWASTRHTGVPDRFTGSGSGTDYILTISSVQAEDLALYYCQQH YSTPYTFGGGTKLEIK or a sequence at least 90% identical thereto;
f. a V_{H} according to the sequence of amino acid QVQLKQSGPSLVQPSQSLSITCTVSGFSLTTYGVHWVRQSPGKGLE WLGVIWRGGSTDYNAGFMSRLSITKDNSKSQVFFKMNSLQADDT AIYYCAKNSYYYGGNYYFDYWGQGTTLTVSS or a sequence at least 90% identical thereto, and a V_{L} according to the amino acid sequence of DVVMTQTPFSLPVSLGDQASISCRSSQSLVHSNGNTHLHWYLQKP GQSPKLLIYKVSNRFSGVPDRFSGSGSGTDFTLKISRVEAEDLGVYF CSQSTHVPPTFGGGTKLEIK or a sequence at least 90% identical thereto;
g. a V_{H} according to the amino acid sequence of QVQLKQSGPSLVQPSQSLSINCTVSGFSLTTYGVHWLRQSPGKGLE WLGVIWRGGSTDYNAAFMSRLSITKDNSKSQVFFKMNSLQADDT AIYYCAKPYYVYGAMDYWGQGTSVTVSS or a sequence at least 90% identical thereto, and a V_{L} according to the amino acid sequence of DVLMTQTPLSLPVSLGDQASISCRSSESIVHSNGNTYLEWFLQKPG QSPKLLIYKVSNRFSGVPDRFSGSGSGTDFTLKISRVEAEDLGVYYC FQGSHVPLTFGAGTKLEIK or a sequence at least 90% identical thereto;
h. a V_{H} according to the amino acid sequence of QVQLQESGPGLVKPSETLSLTCAVSGGSLSSDYWTWIRQPAGKGL EWIGRIFATGTTNYNPSLKSRVTMSIDTSKNQFSLKLNSVTAADTA VYYCARDDWYFDLWGRGTLVTVSS or a sequence at least 90% identical thereto, and a V_{L} according to the amino acid sequence of EIVMTQSPATLSVSPGERATLSCRASQSVSNNLAWYQERPGQAPRL LIYGASTRTTGIPARFSGSGSGTEFILTISSLQPEDFAIYYCQQYNEW PWTFGQGTKVEIK or a sequence at least 90% identical thereto;
i. a V_{H} according to the amino acid sequence of QVQLQESGPGLVKPSETLSLTCTVSGGSISSDYWSWIRQPAGKRLE WIGRIFTSGSTNYNPSLKSRVTMSVDTSKNQFSLKLNSLTAADTAV YYCARDDWYFDLWGRGTLVTVSS or a sequence at least 90% identical thereto, and a V_{H} according to the amino acid sequence of EIVMTQSPATLSVSPGERATLSCRASQSVSNNLAWYQQKPGQAPR FLIYGASTRATGMPARFSGSGSGTEFTLTISSLQSEDFAVYFCQQYN EWPWTFGQGTKVEIK or a sequence at least 90% identical thereto;
j. a V_{H} according to the amino acid sequence of QVQLQESGPGLVKPSETLSLTCTVSGGSISRDYWSWVRQPAGKGL EWIGRIFTNGVTNYNPSLKSRVTMSVDTSKTQFSLKLSSVTAADTA VYFCARDDWYLDLWGRGTLVTVSS or a sequence at least 90% identical thereto, and a V_{L} according to the amino acid sequence of EIVMTQSPATLSVSPGERATLSCRASQSVSNNLAWYQQTPGQAPRF LIYGASTGATGIPARFSGSGSGTEFTLTITSLQSEDFAVYFCQQYND WPWTFGQGTKVEIK or a sequence at least 90% identical thereto;
k. a V_{H} according to the amino acid sequence of QVQMKQSGPGLVQPSQSLSITCTVSGFSLTTYGVHWVRQSPGKGL EWLGVIWSGGSTYYNAAFISRLSISKDNSKSQVIFKMNSLQVDDTA IYYCARWDGYSFYYGMDYWGQGTSVTVSS or a sequence at least 90% identical thereto, and a V_{L} according to the amino acid sequence of QAVVTQESALTTSPGETVTLTCRSSTGAVTTSNYANWVQEKPDHL FTGLIGSTNNRAPGVPARFSGSLIGDKAALIITGAQTEDEARYFCAL WYSNHLVFGGGTKLTVL or a sequence at least 90% identical thereto; or
l. a V_{H} according to the amino acid sequence of QVQLKQSGPGLVQPSQSLSITCTVSGFSLTSYGVHWVRQSPGKGLE WLGVIWSGGSTDYNAAFISRLSISKDNSKSQVFFKMNSLQADDTAI YYCARWDGDSFYYAMDYWGQGTSVTVSS or a sequence at least 90% identical thereto, and a V_{H} according to the amino acid sequence of QAVVIQESALTTSPGETVTLTCRSSTGAVTTSNYASWVQEKPDHLF IGLIGGTNNRAPGVPARFSGSLIGDKAALTITGAQTEDEAIYFCALW YSNHLVFGGGTKLTVL or a sequence at least 90% identical thereto.

In embodiments, the antibody is linked to a therapeutic agent. For example, the therapeutic agent is a toxin, a radiolabel, a siRNA, a small molecule, or a cytokine.

In embodiments, the antibody is a single chain variable fragment (scFv).

Aspects of the invention are further drawn to a nucleic acid encoding the antibody described herein.

Still further, aspects of the invention are drawn to a vector comprising the nucleic acid described herein.

Still further, aspects of the invention are drawn to a cell comprising the nucleic acid described herein.

Aspects of the invention are further drawn to a cell producing the monoclonal antibody described herein.

Aspects of the invention are further drawn to a kit comprising the antibody described herein. In embodiments, the kit comprises a syringe, needle, or applicator for administration of the at least one antibody to a subject; and instructions for use.

Aspects of the invention are further drawn to an isolated multi-specific antibody comprising the monoclonal antibody described herein or a fragment thereof and at least one additional monoclonal antibody or fragment thereof.

In embodiments, the antibody is a bi-specific antibody.

In embodiments, the at least one additional antigen-binding fragment has specificity to a molecule on an immune cell. For example, the molecule is selected from the group consisting of B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, and KIR.

In embodiments, the monoclonal antibody or fragment thereof and the at least one additional antibody or fragment thereof is independently selected from a Fab fragment, a single-chain variable fragment (scFv), or a single-domain antibody.

In embodiments, the antibody is an IL-10 receptor blocking antibody or an IL-10 blocking antibody.

Aspects of the invention are further drawn to a pharmaceutical composition comprising one or more compositions as described herein, and a pharmaceutically acceptable carrier or excipient.

In embodiments, the pharmaceutical composition further comprises at least one additional therapeutic agent. For example, the therapeutic agent is a toxin, a radiolabel, a siRNA, a small molecule, or a cytokine.

Still further, aspects of the invention are drawn to a nanoparticle. In embodiments, the nanoparticles comprise a CD40 agonist antibody, an antibody specific for IL-10 or IL-10R, and monophosphoryl lipid A (MPL).

In embodiments, the CD40 agonist antibody is on the surface of the nanoparticle.

In embodiments, MPL is inside the nanoparticle.

In embodiments, the CD40 agonist antibody is selected from the group consisting of FGK45, CP-870,984, CP-870,983, APX005M, dacetuzumab, and ChiLob 7/4.

In embodiments, the IL-10 antibody is an IL-10 blocking antibody or an IL-10 receptor blocking antibody. For example, the IL-10 or IL-10R antibody is selected from the group consisting of 49G08_HIL2, 49G08, 48C11, 49H07, 48C06, 53D07, 53H08, 42B02, 43C05, 43F05, 15A02, and 21B08.

Aspects of the invention are further drawn to an engineered cell. In embodiments, the engineered cell comprises a chimeric antigen receptor. In embodiments, the chimeric antigen receptor comprises an extracellular ligand binding domain that is specific for Interleukin-10 (IL-10) or IL-10R as described herein.

In embodiments, the extracellular ligand binding domain comprises an antibody or fragment thereof.

In embodiments, the cell comprises a T cell, an NK cell, or an NKT cell. For example, the T cell is CD4+, CD8+, CD3+ pan T cells, or any combination thereof.

Still further, aspects of the invention are drawn to methods of detecting the presence of a cancer in a sample. In embodiments, method comprises contacting the sample with the monoclonal antibody described herein or the multi-specific antibody described herein; and detecting the presence or absence of an antibody-antigen complex, thereby detecting the presence of IL-10 in the sample.

In embodiments, contacting comprises immunohistochemistry. For example, immunohistochemistry comprises precipitation, immunofluorescence, western blot, or ELISA.

In embodiments, the sample is whole blood, a blood component, a body fluid, a biopsy, a tissue, serum or one or more cells.

In embodiments, the sample comprises a normal sample or a cancerous sample.

In embodiments, the one or more cells comprise an in vitro culture. For example, the one or more cells comprise IL-10-expressing cells.

In embodiments, the sample is an in vitro sample.

In embodiments, the method further comprises the step of obtaining a sample from a subject.

In embodiments, the cancer expresses IL-10. For example, IL-10 comprises full-length IL-10.

In embodiments, the cancer comprises a solid tumor. For example, the solid tumor comprises a melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor.

Aspects of the invention are further drawn to a method of treating cancer in a subject. In embodiments, the method comprises contacting a sample obtained from a subject with a monoclonal antibody described herein or the multispecific antibody described herein; detecting the presence or absence of an antibody-antigen complex, wherein the presence of an antibody-antigen complex indicates the presence of cancer in the subject; and administering to the subject an anticancer agent, thereby treating cancer in the subject.

Aspects of the invention are further drawn to a method of treating cancer in a subject. In embodiments, the method comprises administering to a subject in need thereof a monoclonal antibody, a multispecific antibody, a nanoparticle, or an engineered cell, as described herein.

In embodiments, the composition is administered locally, such as intratumorally.

In embodiments, the method further comprises administering to the subject an anti-cancer agent.

Still further, aspects of the invention are drawn to a method of decreasing metastasis in a subject. In embodiments, the method comprises administering to a subject in need thereof a monoclonal antibody, a multispecific antibody, a nanoparticle, or an engineered cell, as described herein.

In embodiments, the subject is afflicted with a cancer. For example, the cancer expresses IL-10. For example, the cancer comprises a solid tumor. For example, the solid tumor comprises a melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor.

Other objects and advantages of this invention will become readily apparent from the ensuing description.

### BRIEF DESCRIPTION OF THE FIGURES

FIG. 1 provides data showing poor binding of αIL-10R candidate to the cynomolgus IL10R. 100× difference in binding affinity prohibits preclinical toxicology assessment.

FIG. 2 represents an evaluation of backup molecules.

FIG. 3 provides sequences showing different nonhuman primate (NHP) species for preclinical toxicology assessment. Sequence similarity above 94% and most mutations are conserved amongst. Similar binding deficiency as for cynomolgus IL10R observed with African green monkey IL10R.

FIG. 4 provides data showing humanized αIL10 candidate blocks STAT3 signaling pathway more efficiently than the benchmark.

FIG. 5 provides data showing the binding affinity of humanized the αIL10 candidate.

FIG. 6 illustrates the humanized αIL10 candidate performs similarly to the benchmark in DC activation assay. MAB2171: anti-human IL-10 (R&D); MC: Maturation cocktail (GM-CSF, IL-4, IL-1b, IL-6, TNFa, PGE-2); MPL: monophosphoryl lipid A, 10 μg/mL; LPS: lipopolysaccharide, 0.1 μg/mL.

FIG. 7 provides data showing optimization of h49G08. 49G08 CDR-H2 contains an aspartate isomerization site in CDRH2; the motif is not present in the germline. D53E mutant was generated to engineer out liability site. hu49G08_D53E variant passed forced stress developability assessment and is currently being evaluated for manufacturability

FIG. 8 provides a schematic showing the generation of tolerogenic and immunogenic dendritic cells from monocytic precursors (adapted from Hubo et al, Frontiers Immunol 4, 82 (2013)).

FIG. 9 provides a schematic showing exemplary animal studies, including humanized mouse models to test in vivo efficacy of IL10/IL10R mAb lead candidates (adapted from The Jackson Laboratory).

FIG. 10 provides data showing the validation of the therapeutic concept with mouse tool mAbs. The validation included syngeneic B16F10 melanoma model, bilateral tumor injection (treatment on one side), and an injection of Monophosphoryl Lipid A (MPL), CD40 agonist Ab, IL-10R blocking Ab, and PD-1 blocking Ab.

FIG. 11 provides a schematic showing the IL-10R & IL-10 antibody discovery strategy.

FIG. 12 shows a schematic of the dendritic cell activation assay. The assay included healthy donor PBMCs, differentiation of monocyte-derived dendritic cells (moDCs), and 48 h stimulation with 0.1 μg/mL LPS and IL-10R1/IL-10 mAbs.

FIG. 13 shows data for the IL-10 antibodies: Mouse Dendritic Cell Activation Assay. Stimulation with LPS in the presence of anti-IL-10 antibodies (0.01-100 μg/ml). Controls: anti-mouse IL-10R mAb (1B1.3A), anti-mouse IL-10 mAb (JES5-2A5). TDI IL-10 antibodies show minimal or no activity in mouse DC assay.

FIG. 14 provides the IL-10R/IL-10 top hits and candidate selection.

FIG. 15 shows graphs of the effects of treatments on tumor size. Panel A provides a schematic depicting the treatment schedule. Panel B provides graph data depicting tumor growth comparing MPL and αCD40 with constituent monotherapies. Panel C provides graph data depicting growth of treated and distant tumors upon addition of αPD-1. Panel D provides data depicting viability of B16F10 cells treated in vitro with MPL, αCD40, or gemcitabine (positive control) for 72 h. Panel E provides data showing bone marrow-derived DCs treated for 16 h and incubated with FITC-labeled latex beads. Panel F provides data depicting CD86 MFI in CD11c^{high} cells in tumor-draining lymph node 24 h after treatment. Panels G-I provide graph data showing treatment as in (Panel A) with treated tumor varied as shown.

FIG. 16 provides graph data showing individual B16F10 tumor growth curves in wild type (WT) and Rag1^{-/-} mice.

FIG. 17 provides graph data showing the efficacy of αCD40/MPL/αIL10R (CMI) is retained at the treated tumor in Rag1^{-/-} mice across the depletions shown, but not in Rag2^{-/-}gc^{-/-} mice. Depletions conformed with flow cytometry. Blue, isotype/control; red, CMI treated.

FIG. 18 shows various bar graphs. Panel A provides graph data depicting the viability of B16F10 cells treated in vitro with αIL10R-containing regimens, or gemcitabine (positive control) for 72 h. Panel B provides graph data showing multi-analyte profiling of tumor cells harvested at 24 h from mice treated with MPL, αCD40, MPL+αCD40, and CMI.

FIG. 19 shows photographs of microscopy images. Panel A provides Hemotoxylin & Eosin staining data showing intratumoral αCD40/MPL/αIL10R (CMI) causes melanin-containing cells with a dendritic morphology to accumulate in the T-cell-rich periarteriolar lymphoid sheath (PALS) of the spleen 24 h after treatment. Such cells were not detected in control mice treated with αCD40+MPL. Panel B provides immunofluorescence assay data showing immunofluorescent labelling of CD4 (green), foxp3 (a regulatory T cell marker, yellow), and CD8 (red) at 6 weeks after treatment with CM (top panels) or CMI (bottom panels).

FIG. 20 Panel A provides data showing expression profiling on distant tumors in bilateral tumor-bearing mice after 1 week of CMP treatment. Shown are transcriptional changes induced by αPD-1 monotherapy or CMP therapy relative to isotype control. Panel B provides heatmap data generated using unsupervised clustering based on annotated gene sets from the Molecular Signatures Database defined as genes upregulated in effector-versus-exhausted CD8+ T cells (GAGE FDR q-value 7.35E-11). Panel C provides flow cytometry data depicting distant tumors from bilateral B16F10 tumor-bearing mice for fraction of PD-1^{high}Eomes^{high} terminally exhausted CD8+ T cells 1 week after isotype or CMP treatment. Panel D provides data showing PD-1^{high} fraction of CD8+ T cells in distant tumors in response to CMP and each constituent monotherapy (in contour plots y-axis, CD8; x-axis, PD-1). Panel E provides graph data showing PD-1^{high} fraction of CD8+ T cells in distant tumors at 6 weeks with isotype or CMP. Panel F provides data depicting the quantification of endogenous gp100-specific CD8+
cells relative to the total CD8+ T-cell population from B16F10 tumor-bearing mice after 1 week of CMP treatment. T cells were stained with H2-Db MHC-I multimers bearing peptides from the melanoma differentiation antigen gp100.

FIG. 21 shows bar graphs. Panel A provides graph data showing human monocyte-derived dendritic cells were treated with isotype, LPS, or α-human CD40 and then assessed by multi-analyte profiling of the supernatant. Shown is secretion of IL10. Panels B-C provide graph data showing flow cytometric quantification of APC activation markers CD80 and CD86 after treatment with LPS, α-human CD40, α-human IL10R, and a combination of both. LPS, lipopolysaccharide (positive control); MFI, median fluorescence intensity.

FIG. 22 provides data showing the binding affinity of 49G08_HIL2. K_{D} (equilibrium binding constant) was determined using surface plasmon resonance (SPR). Samples were captured onto a Cl sensor chip to minimize avidity effects at three different surface densities, and replicated three times.

FIG. 23 provides data showing 49G08_HIL2 is not prone to isomerization. 49G08_HIL2 was stressed for 24 hr at pH3.5 and then dialyzed in PBS. Peptide digestion followed by MS analysis revealed that less than 1% of 49G08_HIL2 isomerizes in acidic stress conditions. Panel A provides data showing in silco sequence analysis. Panel B provides data showing 49G08 CDR-H2 contains an aspartate isomerization site in CDRH2, and that the motif is not present in the germline. Panel C provides mass spectrometry data showing that less than 1% of 49G08_HIL2 isomerizes in acidic stress conditions.

FIG. 24 provides data showing the productivity assessment of 49G08_HIL2. A two-step purification process (Protein A and AEX) was applied, and residual host cell protein and DNA levels were tested for. Panel A provides data showing the productivity of 49G08_HIL2. Panel B provides data showing SDS-Page results of U1694805G0-1 after AEX (anion exchange chromatography) purification. Panel C provides data showing SEC-HPLC (size exclusion-high performance liquid chromatography) results.

FIG. 25 provides data showing the developability assessment of 49G08_HIL2.

### DETAILED DESCRIPTION OF THE INVENTION

Aspects of the invention are drawn to antibodies that specifically bind to Interleukin-10 (IL-10). The antibodies can detect the IL-10 protein in cancer cell lines and tumor samples in which the full-length receptor is expressed using a variety of techniques, including immunoblotting, immunofluorescence and immunoprecipitation. The antibodies can detect the cleaved/shed IL-10 extracellular domain in serum samples of patients with full-length IL-10-expressing tumors. Thus, the clinical utility of these antibodies includes, but is not limited to, the diagnosis of full-length IL-10 expressing tumors, the monitoring response to treatment and early identification of tumor progression and/or relapse. These antibodies also can be utilized in various forms of immunotherapy in combination with other targeted agents.

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to refer to approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

Interleukin 10 (IL-10) is a cytokine with pleiotropic effects in immunoregulation and inflammation. It is produced by mast cells, counteracting the inflammatory effect that these cells have at the site of an allergic reaction. While it can inhibit the synthesis of pro-inflammatory cytokines such as IFN-γ, IL-2, IL-3, TNFα and GM-CSF, IL-10 is also stimulatory towards certain T cells and mast cells and stimulates B-cell maturation, proliferation and antibody production. IL-10 can block NF-κB activity and can be involved in the regulation of the JAK-STAT signaling pathway. It can also induce the cytotoxic activity of CD8+ T-cells and the antibody production of B-cells, and it can suppress macrophage activity and tumor-promoting inflammation. The regulation of CD8+ T-cells is dose-dependent, wherein higher doses induce stronger cytotoxic responses.

Human IL-10 is a homodimer with a molecular mass of 37 kDa, wherein each 18.5 kDa monomer comprises 178 amino acids, the first 18 of which comprise a signal peptide, and two cysteine residues that form two intramolecular disulfide bonds. The IL-10 dimer becomes biologically inactive upon disruption of the non-covalent interactions between the two monomer subunits.

As used herein, the terms “IL-10”, “IL-10 polypeptide(s), “IL-10 molecule(s)”, “IL-10 agent(s)” and the like can refer to, for example, human and non-human IL-10-related polypeptides, including but not limited to homologs, variants (including muteins), and fragments thereof, as well as IL-10 polypeptides having, for example, a leader sequence (e.g., the signal peptide), and modified versions of the foregoing. In other embodiments, IL-10, IL-10 polypeptide(s), and IL-10 agent(s) can be agonists.

As used herein, the terms “homolog” or “antibody homolog” can refer to any molecule which specifically binds IL-10 or IL-10R as described herein. An antibody homolog can include a native or recombinant antibody, whether modified or not, portions of antibodies that retain the biological properties of interest, such as binding IL-10 or IL-10R, a single chain antibody, a polypeptide carrying one or more CDR regions and so on. The amino acid sequence of the homolog need not be identical to that of the naturally occurring antibody, but can be altered or modified to carry substitute amino acids, inserted amino acids, deleted amino acids, amino acids other than the twenty normally found in proteins and so on to obtain a polypeptide with enhanced or other beneficial properties.

As used herein, the term “variant” can refer to a molecule that shows significant structural identity with a reference molecule but can differ structurally from the reference molecule. In embodiments, a variant polypeptide or nucleic acid can differ from a reference polypeptide or nucleic acid as a result of one or more differences in amino acid or nucleotide sequence.

As used herein, the terms “fragment” or “antibody fragment” can refer to a portion of an intact or a full-length chain or an antibody, for example the target binding or variable region. Examples of antibody fragments include, but are not limited to, F_{ab}, F_{ab′}, F_{(ab′)2} and Fᵥ fragments.

The IL-10 receptor, a type II cytokine receptor, consists of alpha and beta subunits, which can be referred to as R1 and R2, respectively. Receptor activation requires binding to both alpha and beta. One homodimer of an IL-10 polypeptide binds to the alpha subunit and the other homodimer of the same IL-10 polypeptide binds to the beta.

The utility of recombinant human IL-10 is frequently limited by its relatively short serum half-life, which can be due to, for example, renal clearance, proteolytic degradation and monomerization in the blood stream. As a result, various approaches have been explored to improve the pharmacokinetic profile of IL-10 without disrupting its dimeric structure and thus adversely affecting its activity. Pegylation of IL-10 results in improvement of certain pharmacokinetic parameters (e.g., serum half-life) and/or enhancement of activity.

Aspects of the invention provide isolated monoclonal antibodies specific to IL-10 and/or IL-10R. The term “isolated”, for example as used herein with respect to cells, proteins, and/or nucleic acids, such as DNA or RNA, can refer to molecules separated from other cells, proteins, and/or DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and cannot be found in the natural state. “Isolated” can also refer to cells or polypeptides which are isolated from other cellular proteins or tissues. Isolated polypeptides can include both purified and recombinant polypeptides.

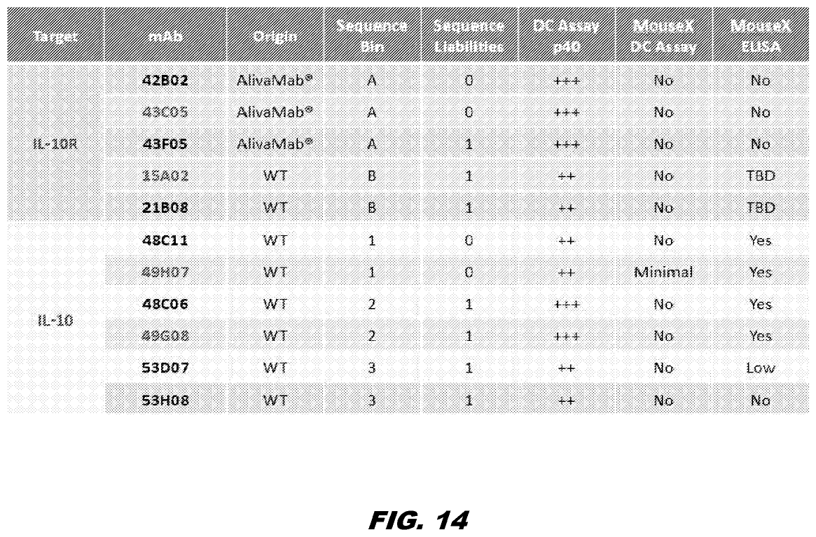

Unique recombinant monoclonal IL-10 and IL-10R antibodies are described herein. These include clones specific for IL-10 (e.g., 49G08_HIL2, 49G08, 48C11, 49H07, 48C06, 53D07, 53H08), and clones specific for IL-10R (42B02, 43C05, 43F05, 15A02, and 21B08). See, for example, FIG. 14. “Recombinant”, for example as it pertains to polypeptides (such as antibodies) or polynucleotides, can refer to a form of the polypeptide or polynucleotide that does not exist naturally, a non-limiting example of which can be created by combining polynucleotides or polypeptides that cannot normally occur together.

The nucleic acid and amino acid sequence of the monoclonal IL-10/IL-10R antibodies are provided below. Specifically, the amino acid sequences of the heavy and light chain complementary determining regions (CDRs) of the IL-10 antibodies are underlined (CDR1), underlined and bolded (CDR2), or underlined, italicized, and bolded (CDR3).

TABLE 1
Ab 49G08_H1L2 Variable Region amino
acid sequences

V_{H} chain of Ab_49G08_H1L2
EVQLVESGGGLVQPGGSLRLSCAASGFTFSDYYMYWVR
QAPGKGLEWVSTISDGGSYTYYPDSVKGRFTISRDNAK
NNLYLQMNSLRAEDTAVYYCARGYYYGLFTYWGQGTLV
TVSS
V_{L} chain of Ab_49G08_H1L2
DIQMTQSPSSLSASVGDRVTITCRASQDVNTGLNWYQQ
KPGQAPKLLIYWASSRQSGVPSRESGSGSGTDFTLTIS
SLQPEDFATYYCQQHYSTPYTFGQGTKLEIK

TABLE 2
Ab 49G08 Variable Region amino acid sequences

V_{H} chain of Ab 49G08
EVQLVESGGGLVKPGGSLKLSCAASGFTFSDYYMYWVRQTPEKRLE
WVATISDGGSYTYYPDSVKGRFTISRDNAKNNLYLQMSSLKSEDTA
MFYCARGYYYGLFTYWGQGTLVTVSA
V_{L} chain of Ab 49G08
DIVMTQSHKFMSTSVGDRVSITCKASQDVNTGVAWYQQKPGQSPKL
LIYWASTRHTGVPDRFTGSGSGTDYILTISSVQAEDLALYYCQQHY
STPYTFGGGTKLEIK

TABLE 3
Ab 48C11 Variable Region amino acid sequences

V_{H} chain of Ab 48C11
EVQLQQSGPDLVKPGASVRMSCKASGYTFTNYVMHWVKQRPGQGL
EWIGYISPYNDGTKYNEKFKGKATLTSDKSSSTAYMELNSLTSED
SAVYYCAGGDYSYAMDYWGQGTSVTVSS
V_{L} chain of Ab 48C11
DIQMTQSPASLSVSVGETVTITCRVSENIYSNLAWYHQKQGKSPQ
LLVYAATNLADGVPSRESGSKSGTQYSLKISSLQSEDFGSYYCQH
EWGTLTFGAGTKLELK

TABLE 4
Ab 49H07 Variable Region amino acid sequences

V_{H} chain of Ab 49H07
EVQLQQSGPDLVKPGASVKMSCKASGYTFTHYVMHWVKQKPGQGL
EWIGYISPYNDGIKYNEKFKGKATLTSDKSSSTAYMELSSLTSED
SAVYYCAGGDYSYAMDYWGQGTSVTVSS
V_{L} chain of Ab 49H07
DIQMTQSPASLSVSVGETVTITCRVSENIYSNLAWYQQKQGKSPQ
LLVYAATNLADGVSSRESGSVSGTQYSLKINSLQSEDFGSYYCQH
EWGTLTFGAGTKLELK

TABLE 5
Ab 48C06 Variable Region amino acid sequences

V_{H} chain of Ab 48C06
EVQLVESGGGLVKPGGSLKLSCAASGFTESDYYIYWVRQT
PEKRLEWVATISDGGSYTYYPDSVKGRFTISRDNAKNNLY
LQMSSLKSEDTAMYYCARGYYYGLFTYWGQGTLVTVSA
V_{L} chain of Ab 48C06
DIVMTQSHKFMSTSVGDRVSITCKASQDVSTGVAWYRQKP
GRSPKLLIYWASTRHTGVPDRFTGSGSGTDYILTISSVQA
EDLALYYCQQHYSTPYTFGGGTKLEIK

TABLE 6
Ab 53D07 Variable Region amino acid sequences

V_{H} chain of Ab 53D07
QVQLKQSGPSLVQPSQSLSITCTVSGFSLTTYGVHWVRQS
PGKGLEWLGVIWRGGSTDYNAGFMSRLSITKDNSKSQVFF
KMNSLQADDTAIYYCAKNSYYYGGNYYFDYWGQGTTLTVS
S
V_{L} chain of Ab 53D07
DVVMTQTPFSLPVSLGDQASISCRSSQSLVHSNGNTHLHW
YLQKPGQSPKLLIYKVSNRFSGVPDRFSGSGSGTDFTLKI
SRVEAEDLGVYFCSQSTHVPPTFGGGTKLEIK

TABLE 7
Ab 53H08 Variable Region amino acid sequences

V_{H} chain of Ab 53H08
QVQLKQSGPSLVQPSQSLSINCTVSGESLTTYGVHWLRQS
PGKGLEWLGVIWRGGSTDYNAAFMSRLSITKDNSKSQVFF
KMNSLQADDTAIYYCAKPYYVYGAMDYWGQGTSVTVSS
V_{L} chain of Ab 53H08
DVLMTQTPLSLPVSLGDQASISCRSSESIVHSNGNTYLEW
FLQKPGQSPKLLIYKVSNRFSGVPDRFSGSGSGTDFTLKI
SRVEAEDLGVYYCFQGSHVPLTFGAGTKLEIK

TABLE 8
Ab 42B02 Variable Region amino acid sequences

VH chain of Ab 42B02
QVQLQESGPGLVKPSETLSLTCAVSGGSLSSDYWTWIRQP
AGKGLEWIGRIFATGTTNYNPSLKSRVTMSIDTSKNQFSL
KLNSVTAADTAVYYCARDDWYFDLWGRGTLVTVSS
V_{L} chain of Ab 42B02
EIVMTQSPATLSVSPGERATLSCRASQSVSNNLAWYQERP
GQAPRLLIYGASTRTTGIPARFSGSGSGTEFILTISSLQP
EDFAIYYCQQYNEWPWTFGQGTKVEIK

TABLE 9
Ab 43C05 Variable Region amino acid sequences

V_{H} chain of Ab 43C05
QVQLQESGPGLVKPSETLSLTCTVSGGSISSDYWSWIRQP
AGKRLEWIGRIFTSGSTNYNPSLKSRVTMSVDTSKNQFSL
KLNSLTAADTAVYYCARDDWYFDLWGRGTLVTVSS
V_{L} chain of Ab 43C05
EIVMTQSPATLSVSPGERATLSCRASQSVSNNLAWYQQKP
GQAPRFLIYGASTRATGMPARESGSGSGTEFTLTISSLQS
EDFAVYFCQQYNEWPWTFGQGTKVEIK

TABLE 10
Ab 43F05 Variable Region amino acid sequences

V_{H} chain of Ab 43F05
QVQLQESGPGLVKPSETLSLTCTVSGGSISRDYWSWVRQP
AGKGLEWIGRIFTNGVTNYNPSLKSRVTMSVDTSKTQFSL
KLSSVTAADTAVYFCARDDWYLDLWGRGTLVTVSS
V_{L} chain of Ab 43F05
EIVMTQSPATLSVSPGERATLSCRASQSVSNNLAWYQQTP
GQAPRFLIYGASTGATGIPARESGSGSGTEFTLTITSLQS
EDFAVYFCQQYNDWPWTFGQGTKVEIK

TABLE 11
Ab 15A02 Variable Region amino acid sequences

V_{H} chain of Ab 15A02
QVQMKQSGPGLVQPSQSLSITCTVSGFSLTTYGVHWVRQS
PGKGLEWLGVIWSGGSTYYNAAFISRLSISKDNSKSQVIF
KMNSLQVDDTAIYYCARWDGYSFYYGMDYWGQGTSVTVSS
V_{L} chain of Ab 15A02
QAVVTQESALTTSPGETVTLTCRSSTGAVTTSNYANWVQE
KPDHLFTGLIGSTNNRAPGVPARFSGSLIGDKAALIITGA
QTEDEARYFCALWYSNHLVEGGGTKLTVL

TABLE 12
Ab 21B08 Variable Region amino acid sequences

V_{H} chain of Ab 21B08
QVQLKQSGPGLVQPSQSLSITCTVSGFSLTSYGVHWVRQS
PGKGLEWLGVIWSGGSTDYNAAFISRLSISKDNSKSQVFF
KMNSLQADDTAIYYCARWDGDSFYYAMDYWGQGTSVTVSS
V_{L} chain of Ab 21B08
QAVVIQESALTTSPGETVTLTCRSSTGAVTTSNYASWVQE
KPDHLFIGLIGGTNNRAPGVPARFSGSLIGDKAALTITGA
QTEDEAIYFCALWYSNHLVFGGGTKLTVL

The amino acid sequences of the heavy and light chain complementary determining regions of the IL-10/IL-10R antibodies are shown below:

TABLE 13A

| 49G08_H1L2 CDR VH | Protein Sequence |
|---|---|
| VH CDR-1 | GFTFSDYY |
| VH CDR-2 | ISDGGSYT |
| VH CDR-3 | ARGYYYGLFTY |

TABLE 13B

| 49G08_H1L2 CDR VL | Protein Sequence |
|---|---|
| VL CDR-1 | QDVNTG |
| VL CDR-2 | WAS |
| VL CDR-3 | QQHYSTPYT |

TABLE 14A

| 49G08 CDR VH | Protein Sequence |
|---|---|
| VH CDR-1 | GFTFSDYY |
| VH CDR-2 | ISDGGSYT |
| VH CDR-3 | ARGYYYGLFTY |

TABLE 14B

| 49G08 CDR VL | Protein Sequence |
|---|---|
| VL CDR-1 | QDVNTG |
| VL CDR-2 | WAS |
| VL CDR-3 | QQHYSTPYT |

TABLE 15A

| 48C11 CDR VH | Protein Sequence |
|---|---|
| VH CDR-1 | GYTFTNYV |
| VH CDR-2 | ISPYNDGT |
| VH CDR-3 | AGGDYSYAMDY |

TABLE 15B

| 48C11 CDR V_{L} | Protein Sequence |
|---|---|
| VL CDR-1 | ENIYSN |
| VL CDR-2 | AAT |
| VL CDR-3 | QHFWGTLT |

TABLE 16A

| 49H07 CDR VH | Protein Sequence |
|---|---|
| VH CDR-1 | GYTFTHYV |
| VH CDR-2 | ISPYNDGI |
| VH CDR-3 | AGGDYSYAMDY |

TABLE 16B

| 49H07 CDR V_{L} | Protein Sequence |
|---|---|
| VL CDR-1 | ENIYSN |
| VL CDR-2 | AAT |
| VL CDR-3 | QHFWGTLT |

TABLE 17A

| 48C06 CDR VH | Protein Sequence |
|---|---|
| VH CDR-1 | GFTFSDYY |
| VH CDR-2 | ISDGGSYT |
| VH CDR-3 | ARGYYYGLFTY |

TABLE 17B

| 48C06 CDR V_{L} | Protein Sequence |
|---|---|
| VL CDR-1 | QDVSTG |
| VL CDR-2 | WAS |
| VL CDR-3 | QQHYSTPYT |

TABLE 18A

| 53D07 CDR VH | Protein Sequence |
|---|---|
| VH CDR-1 | GFSLTTYG |
| VH CDR-2 | IWRGGST |
| VH CDR-3 | AKNSYYYGGNYYFDY |

TABLE 18B

| 53D07 CDR V_{L} | Protein Sequence |
|---|---|
| VL CDR-1 | QSLVHSNGNTH |
| VL CDR-2 | KVS |
| VL CDR-3 | SQSTHVPPT |

TABLE 19A

| 53H08 CDR VH | Protein Sequence |
|---|---|
| VH CDR-1 | GFSLTTYG |
| VH CDR-2 | IWRGGST |
| VH CDR-3 | AKPYYVYGAMDY |

TABLE 19B

| 53H08 CDR V_{L} | Protein Sequence |
|---|---|
| VL CDR-1 | ESIVHSNGNTY |
| VL CDR-2 | KVS |
| VL CDR-3 | FQGSHVPLT |

TABLE 20A

| 42B02 CDR VH | Protein Sequence |
|---|---|
| VH CDR-1 | GGSLSSDY |
| VH CDR-2 | IFATGTT |
| VH CDR-3 | ARDDWYFDL |

TABLE 20B

| 42B02 CDR V_{L} | Protein Sequence |
|---|---|
| VL CDR-1 | QSVSNN |
| VL CDR-2 | GAS |
| VL CDR-3 | QQYNEWPWT |

TABLE 21A

| 43C05 CDR VH | Protein Sequence |
|---|---|
| VH CDR-1 | GGSISSDY |
| VH CDR-2 | IFTSGST |
| VH CDR-3 | ARDDWYFDL |

TABLE 21B
43C05 CDR VL

| | Protein Sequence |
|---|---|
| VL CDR-1 | QSVSNN |
| VL CDR-2 | GAS |
| VL CDR-3 | QQYNEWPWT |

TABLE 22A
43F05 CDR VH

| | Protein Sequence |
|---|---|
| VH CDR-1 | GGSISRDY |
| VH CDR-2 | IFTNGVT |
| VH CDR-3 | ARDDWYLDL |

TABLE 22B
43F05 CDR VL

| | Protein Sequence |
|---|---|
| VL CDR-1 | QSVSNN |
| VL CDR-2 | GAS |
| VL CDR-3 | QQYNDWPWT |

TABLE 23A
15A02 CDR VH

| | Protein Sequence |
|---|---|
| VH CDR-1 | GFSLTTYG |
| VH CDR-2 | IWSGGST |
| VH CDR-3 | ARWDGYSFYYGMDY |

TABLE 23B
15A02 CDR VL

| | Protein Sequence |
|---|---|
| VL CDR-1 | TGAVTTSNY |
| VL CDR-2 | STN |
| VL CDR-3 | ALWYSNHLV |

TABLE 24A
21B08 CDR VH

| | Protein Sequence |
|---|---|
| VH CDR-1 | GFSLTSYG |
| VH CDR-2 | IWSGGST |
| VH CDR-3 | ARWDGDSFYYAMDY |

TABLE 24B
21B08 CDR VL

| | Protein Sequence |
|---|---|
| VL CDR-1 | TGAVTTSNY |
| VL CDR-2 | GTN |
| VL CDR-3 | ALWYSNHLV |

The IL-10 antibodies described herein bind to IL-10, and the IL-10R antibodies described herein bind to IL-10R. In one embodiment, the IL-10 antibodies have high affinity and high specificity for IL-10. In one embodiment, the IL-10R antibodies have high affinity and high specificity for IL-10R. In some embodiments, the IL-10 antibodies do not cross react with IL-10R, and the IL-10R antibodies do not cross react with IL-10.

Embodiments feature antibodies that have a specified percentage identity or similarity to the amino acid or nucleotide sequences of the anti-IL-10/anti-IL-10R antibodies described herein. For example, “homology” or “identity” or “similarity” can refer to sequence similarity between two peptides or between two nucleic acid molecules. Homology can be determined by comparing a position in each sequence, which can be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-IL-10 antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-IL-10/anti-IL-10R antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

“Polypeptide” as used herein can encompass a singular “polypeptide” as well as plural “polypeptides,” and can refer to a molecule composed of monomers (amino acids) linearly linked by amide bonds (also known as peptide bonds). The term “polypeptide” can refer to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It can be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In embodiments the alteration results in the substitution of an amino acid with a chemically similar amino acid. Conservative substitution tables providing functionally similar amino acids are well known in the art.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide can be replaced with another amino acid residue from the same side chain family. In another embodiment, a string of amino acids can be replaced with a structurally similar string that differs in order and/or composition of side chain family members.

An “antibody” or “antigen-binding polypeptide” can refer to a polypeptide or a polypeptide complex that specifically recognizes and binds to an antigen, such as IL-10. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. “Specifically binds” or “immunoreacts with” can refer to the antibody reacting with one or more antigenic determinants of the target antigen and does not react with other polypeptides.

The terms “antibody fragment” or “antigen-binding fragment” can refer to a portion of an antibody such as F_{(ab)2}, F_{(ab)2}, F_{ab}′, F_{ab}, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising a VL or VH domain, fragments produced by a Fab expression library, and anti-idiotypic (anti-Id) antibodies.

A “single-chain variable fragment” or “scFv” can refer to a fusion protein of the variable regions of the heavy (V_{H}) and light chains (V_{L}) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85 (16): 5879-5883). In embodiments the regions are connected with a short linker peptide, such as a short linker peptide of about ten to about 25 amino acids. The linker can be rich in glycine for flexibility, as well as serine or threonine for solubility, and can connect the N-terminus of the V_{H} with the C-terminus of the V_{L}, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglubulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains can be joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as kappa or lambda (κ, λ). Each heavy chain class can be bound with a kappa or lambda light chain. The light and heavy chains are covalently bonded to each other, and the “tail” portions of the two heavy chains are bonded to each other by covalent disulfide linkages or non-covalent linkages when the immunoglobulins are generated by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” VH and VL regions, which contain the CDRs, as well as frameworks (FRs) of the IL-10 antibodies are shown in Table 1-Table 11.

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions can adopt a B-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services. (1983); and Chothia and Lesk, J. Mol. Biol. 196:901-917 (1987)).

Where there are two or more definitions of a term which is used and/or accepted within the art, the definition of the term as used herein is intended to include such meanings unless explicitly stated to the contrary. A specific example is the use of the term “complementarity determining region” (“CDR”) to describe the non-contiguous antigen combining sites found within the variable region of both heavy and light chain polypeptides. This region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a CDR will vary depending on the sequence and size of the CDR. Those skilled in the art can routinely determine which residues comprise a CDR given the variable region amino acid sequence of the antibody.

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” can refer to the numbering system set forth by Kabat et al., U.S. Dept. of Health and Human Services, “Sequence of Proteins of Immunological Interest” (1983).

As used herein, the term “epitope” can include any protein determinant that specifically binds to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants consist of chemically active surface groupings of molecules such as amino acids or sugar side chains and can have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3).

In one embodiment, the antibodies can be directed to IL-10 (Accession no. P22301).

The amino acid sequence for IL-10 protein comprises:

MHSSALLCCLVLLTGVRASPGQGTQSENSCTHFPGNLPNMLRDLRDAFS
RVKTFFQMKDQLDNLLLKESLLEDFKGYLGCQALSEMIQFYLEEVMPQA
ENQDPDIKAHVNSLGENLKTLRLRLRRCHRFLPCENKSKAVEQVKNAFN
KLQEKGIYKAMSEFDIFINYIEAYMTMKIRN

In one embodiment, the antibodies can be directed to IL-10R (Accession no. Q13651).

The amino acid sequence for IL-10R protein comprises:

MLPCLVVLLAALLSLRLGSDAHGTELPSPPSVWFEAEFFHHILHWTPIP
NQSESTCYEVALLRYGIESWNSISNCSQTLSYDLTAVTLDLYHSNGYRA
RVRAVDGSRHSNWTVTNTRFSVDEVTLTVGSVNLEIHNGFILGKIQLPR
PKMAPANDTYESIFSHFREYEIAIRKVPGNFTFTHKKVKHENFSLLTSG
EVGEFCVQVKPSVASRSNKGMWSKEECISLTRQYFTVTNVIIFFAFVLL
LSGALAYCLALQLYVRRRKKLPSVLLFKKPSPFIFISQRPSPETQDTIH
PLDEEAFLKVSPELKNLDLHGSTDSGFGSTKPSLQTEEPQFLLPDPHPQ
ADRTLGNREPPVLGDSCSSGSSNSTDSGICLQEPSLSPSTGPTWEQQVG
SNSRGQDDSGIDLVQNSEGRAGDTQGGSALGHHSPPEPEVPGEEDPAAV
AFQGYLRQTRCAEEKATKTGCLEEESPLTDGLGPKFGRCLVDEAGLHPP
ALAKGYLKQDPLEMTLASSGAPTGQWNQPTEEWSLLALSSCSDLGISDW
SFAHDLAPLGCVAAPGGLLGSFNSDLVTLPLISSLQSSE

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the equilibrium binding constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ allows for the cancellation of parameters not related to affinity, and is equal to the equilibrium binding constant, K_{D}. (See, generally, Davies et al. (1990) Annual Rev Biochem 59:439-473). An antibody of the present invention can specifically bind to a IL-10 epitope when the equilibrium binding constant (K_{D}) is ≤1 μM, ≤10 μM, ≤10 nM, ≤10 μM, or ≤100 pM to about 1 μM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the Ko is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it can bind to a random, unrelated epitope.

For example, the IL-10 antibody can be monovalent or bivalent, and comprises a single or double chain. Functionally, the binding affinity of the IL-10 antibody is within the range of 10⁻⁵M to 10⁻¹²M. For example, the binding affinity of the IL-10 antibody is from 10⁻⁶ M to 10⁻¹² M, from 10⁻⁷M to 10⁻¹² M, from 10⁻⁸ M to 10⁻¹²M, from 10⁻⁹M to 10⁻¹² M, from 10⁻⁵M to 10⁻¹¹ M, from 10⁻⁶M to 10⁻¹¹M, from 10⁻⁷M to 10⁻¹¹ M, from 10⁻⁸M to 10⁻¹¹ M, from 10⁻⁹M to 10⁻¹¹ M, from 10⁻¹⁰ M to 10⁻¹¹ M, from 10⁻⁵ M to 10⁻¹⁰M, from 10⁻⁶M to 10⁻¹⁰ M, from 10⁻⁷M to 10⁻¹⁰M, from 10⁻⁸ M to 10⁻¹⁰M, from 10⁻⁹M to 10⁻¹⁰ M, from 10⁻⁵ M to 10⁻⁹M, from 10⁻⁶ M to 10⁻⁹M, from 10⁻⁷M to 10⁻⁹M, from 10⁻⁸M to 10⁻⁹M, from 10⁻⁵ M to 10⁻⁸ M, from 10⁻⁶ M to 10⁻⁸ M, from 10⁻⁷M to 10⁻⁸ M, from 10⁻⁵ M to 10⁻⁷M, from 10⁻⁶ M to 10⁻⁷M, or from 10⁻⁵ M to 10⁻⁶M.

An IL-10 protein or IL-10R protein, or a derivative, fragment, analog, homolog or ortholog thereof, can be utilized as an immunogen in the generation of antibodies that immunospecifically bind these protein components. For example, the immunogen can comprise IL10 ECD (Met1-Asn178). For example, the immunogen can comprise IL10R1-huFc (His22-Asn235-huFc) final boost with IL10R1 ECD (His22-Asn235).

As used herein, the term “derivative” can refer to a polypeptide related to a wild type polypeptide, for example by amino acid sequence, structure (e.g., secondary and/or tertiary), activity (e.g., enzymatic activity) and/or function. Derivatives can comprise one or more amino acid variations (e.g., mutations, insertions, and deletions), truncations, modifications, or combinations thereof compared to a wild type polypeptide.

As used herein, the term “analog” can refer a proteinaceous agent (e.g., protein, polypeptide, and antibody) that possesses a similar or identical function as a second proteinaceous agent but does not necessarily comprise a similar or identical amino acid sequence of the second proteinaceous agent, or possess a similar or identical structure of the second proteinaceous agent.

As used herein, an “ortholog” of a protein can refer to a protein of a different species which performs the same or a similar function as the protein it is an ortholog of.

As used herein, the term “immunogen” can refer to a substance, such as a protein, that can induce an immune response in a subject. “Immunogen” can also refer to a protein that is immunologically active in the sense that once administered to a subject (e.g., directly or by administering to the subject a nucleotide sequence or vector that encodes the protein) is able to evoke an immune response of the humoral and/or cellular type directed against that protein.

In embodiments, the immunogen comprises the extracellular domain.

An IL-10 protein or an IL-10R protein or a derivative, fragment, analog, homolog, or ortholog thereof, coupled to a proteoliposome can be utilized as an immunogen in the generation of antibodies that immunospecifically bind these protein components.

Those skilled in the art will recognize that one can determine, without undue experimentation, if a human monoclonal antibody has the same specificity as a human monoclonal antibody of the invention by ascertaining whether the former prevents the latter from binding to IL-10. For example, if the human monoclonal antibody being tested competes with the human monoclonal antibody of the invention, as shown by a decrease in binding by the human monoclonal antibody of the invention, then the two monoclonal antibodies can bind to the same, or to a closely related, epitope.

Another way to determine whether a human monoclonal antibody has the specificity of a human monoclonal antibody of the invention is to pre-incubate the human monoclonal antibody of the invention with the IL-10 or IL-10R protein, with which it is normally reactive, and then add the human monoclonal antibody being tested to determine if the human monoclonal antibody being tested is inhibited in its ability to bind IL-10 or IL-10R. If the human monoclonal antibody being tested is inhibited then, it can have the same, or functionally equivalent, epitopic specificity as the monoclonal antibody of the invention. Screening of human monoclonal antibodies of the invention can be also carried out by utilizing IL-10 and determining whether the test monoclonal antibody is able to neutralize IL-10.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen, which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in the molecules of the population. MAbs contain an antigen binding site that immunoreacts with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is immunized with an immunizing agent to elicit lymphocytes that produce or can produce antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are of interest, or spleen cells or lymph node cells can be used if non-human mammalian sources are of interest. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, for example, myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas will include hypoxanthine, aminopterin, and thymidine (“HAT medium”), which substances prevent the growth of HGPRT-deficient cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, California) and the American Type Culture Collection (Manassas, Virginia). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the hybridoma cells of interest are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that bind specifically to genes encoding the heavy and light chains of murine antibodies). The hybridoma cells as described herein can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “human antibodies” or “fully human antibodies” herein. Human monoclonal antibodies can be prepared by using trioma technique; the human Bcell hybridoma technique (see Kozbor, et al., 1983 Immunol Today 4:72); and the EBV hybridoma technique to produce human monoclonal antibodies (see Cole, et al., 1985 In: MONOCLONAL ANTIBODIES AND CANCER THERAPY. Alan R. Liss. Inc., pp. 7796). Human monoclonal antibodies can be utilized and can be produced by using human hybridomas (see Cote, et al., 1983. Proc Natl Acad Sci USA 80:20262030) or by transforming human Bcells with Epstein Barr Virus in vitro (see Cole, et al., 1985 In: MONOCLONAL ANTIBODIES AND CANCER THERAPY, Alan R. Liss, Inc., pp. 7796).

“Humanized antibodies” can be antibodies from non-human species (such as a mouse) whose light chain and heavy chain protein sequences have been modified to increase their similarity to antibody variants produced in humans. Humanized antibodies are antibody molecules derived from a non-human species antibody that bind the antigen of interest having one or more complementarity determining regions (CDRs) from the non-human species and framework regions from a human immunoglobulin molecule. Often, framework residues in the human framework regions will be substituted with the corresponding residue from the CDR donor antibody to alter, or for example improve, antigen-binding. These framework substitutions are identified by methods well known in the art, e.g., by modeling of the interactions of the CDR and framework residues to identify framework residues important for antigen-binding and sequence comparison to identify unusual framework residues at certain positions. (See, e.g., Queen et al., U.S. Pat. No. 5,585,089; Riechmann et al., Nature 332:323 (1988), which are incorporated herein by reference in their entireties.) For example, the non-human part of the antibody (such as the CDR(s) of a light chain and/or heavy chain) can bind to the target antigen.

Antibodies can be humanized using a variety of techniques known in the art including, for example, CDR-grafting (EP 239.400; PCT publication WO 91/09967; U.S. Pat. Nos. 5,225,539; 5,530,101; and 5,585,089), veneering or resurfacing (EP 592,106; EP 519,596; Padlan, Molecular Immunology 28 (4/5): 489-498 (1991); Studnicka et al., Protein Engineering 7 (6): 805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (such as rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20). Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant can be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the invention further provides any cell or plant comprising a vector that encodes an antibody of the invention, or produces an antibody of the invention.

Human monoclonal antibodies, such as fully human and humanized antibodies, can be prepared by using trioma technique; the human B-cell hybridoma technique (see Kozbor, et al, 1983 Immunol Today 4:72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80:2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using other techniques, including phage display libraries. (See Hoogenboom and Winter, J. Mol. Biol, 227:381 (1991); Marks et al., J. Mol. Biol, 222:581 (1991)). Human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al, Nature 368, 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides the modifications of interest is then obtained as progeny by crossbreeding intermediate transgenic animals containing fewer than the full complement of the modifications. A non-limiting example of such a nonhuman animal is a mouse, and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633.425; 5,569.825; 5,661,016; 5,545,806; 5,814.318; and 5,939,598, which are incorporated by reference herein in their entirety. In addition, companies such as Creative BioLabs (Shirley, NY) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody described herein, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described herein.

These vectors can include liposomes, naked DNA, adjuvant-assisted DNA, gene gun, catheters, etc. Vectors can further include chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used, and include moloney murine leukemia viruses.

DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87:1149 (1990), Adenovirus Vectors (see LeGal LaSalle et al, Science, 259:988 (1993); Davidson, et al, Nat. Genet 3:219 (1993); Yang, et al, J. Virol. 69:2004 (1995) and Adeno-associated Virus Vectors (see Kaplitt, M. G . . . et al, Nat. Genet. 8:148 (1994).

Pox viral vectors introduce the gene into the cell's cytoplasm. Avipox virus vectors result in only a short-term expression of the nucleic acid. Adenovirus vectors, adeno-associated virus vectors, and herpes simplex virus (HSV) vectors can be used for introducing the nucleic acid into neural cells. The adenovirus vector results in a shorter-term expression (about 2 months) than adeno-associated virus (about 4 months), which in turn is shorter than HSV vectors. The vector chosen will depend upon the target cell and the condition being treated. The introduction can be by standard techniques, e.g. infection, transfection, transduction or transformation. Examples of modes of gene transfer include e.g., naked DNA, CaP0₄ precipitation, DEAE dextran, electroporation, protoplast fusion, lipofection, cell microinjection, and viral vectors.

The vector can be employed to target essentially a target cell of interest. For example, stereotaxic injection can be used to direct the vectors (e.g. adenovirus, HSV) to a location of interest. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91:2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways, for example, to detect the presence of IL-10 or IL-10R in a sample. The antibody can also be used to try to bind to and disrupt an IL-10 or IL-10R activity. In an embodiment, the antibodies of the invention are full-length antibodies, containing an Fc region similar to wild-type Fc regions that bind to Fc receptors. In other embodiments, the antibodies of the invention are antibody fragments, such as scFv antibodies.

Techniques can be adapted for the production of single-chain antibodies specific to an antigenic protein of the invention (See e.g., U.S. Pat. No. 4,946,778). In addition, methods can be adapted for the construction of Fab expression libraries (See e.g., Huse, et al, 1989 Science 246:1275-1281) to allow rapid and effective identification of monoclonal Fab fragments with the specificity for a protein or derivatives, fragments, analogs or homologs thereof. Antibody fragments that contain the idiotypes to a protein antigen can be produced by techniques known in the art including, but not limited to: (i) an F(ab′) 2 fragment produced by pepsin digestion of an antibody molecule; (ii) an Fab fragment generated by reducing the disulfide bridges of an F(ab′) 2 fragment; (iii) an Fab fragment generated by the treatment of the antibody molecule with papain and a reducing agent and (iv) Fv fragments.

Heteroconjugate antibodies are also within the scope of the present invention. Heteroconjugate antibodies are composed of two covalently joined antibodies. Such antibodies can, for example, target immune system cells to unwanted cells (see U.S. Pat. No. 4,676,980), and for treatment of infection (See PCT Publication Nos. WO91/00360; WO92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176:1 191-1 195 (1992) and Shopes, J. Immunol., 148:2918-2922 (1992)). Alternatively, an antibody can be engineered that has dual Fc regions and can thereby have enhanced complement lysis and ADCC capabilities. (See Stevenson et al, Anti-Cancer Drug Design, 3:219-230 (1989)).

In embodiments, an antibody of the invention can comprise an Fc variant comprising an amino acid substitution which alters the antigen-independent effector functions of the antibody, such as the circulating half-life of the antibody. Such antibodies exhibit increased or decreased binding to FcRn when compared to antibodies lacking these substitutions, therefore, have an increased or decreased half-life in serum, respectively. Fc variants with improved affinity for FcRn can have longer serum half-lives, and such molecules have useful applications in methods of treating mammals where long half-life of the administered antibody is of interest, e.g., to treat a chronic disease or disorder. In contrast, Fc variants with decreased FcRn binding affinity can have shorter half-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity is of interest can include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In embodiments, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS.

The invention also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al, Science 238:1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages are less stable than other linkages because the disulfide linkage is cleaved in vitro, resulting in less conjugate available. Sulfo-NHS can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al. Proc. Natl. Acad. Sci. USA, 82:3688 (1985); Hwang et al. Proc. Natl Acad. Sci. USA. 77:4030 (1980); and U.S. Pat. Nos. 4,485,045 and 4,544.545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the diameter of interest. Fab′ fragments of the antibody of the present invention can be conjugated to the liposomes as described in Martin et al, J. Biol. Chem., 257:286-288 (1982) via a disulfide-interchange reaction.

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. The invention provides for multispecific antibodies, such as bi-specific antibodies that recognize IL-10 or IL-10R and a second antigen. For example, an antibody or antigen-binding fragment specific to IL-10 can be combined with a second antigen-binding fragment, such as an antigen binding fragment specific to an immune cell, to generate a bispecific antibody. In embodiments, the immune cell can be selected from the group consisting of a T cell, a B cell, a monocyte, a macrophage, a neutrophil, a dendritic cell, a phagocyte, a natural killer cell, an eosinophil, a basophil, and a mast cell. Molecules on the immune cell which can be targeted include, but not limited to, for example, CD3, CD16, CD19, CD28, and CD64. Other non-limiting examples include PD-1, CTLA-4, LAG-3 (also known as CD223), CD28, CD122, 4-1BB (also known as CD137), TIM3, OX-40 or OX40L, CD40 or CD40L, LIGHT, ICOS/ICOSL, GITR/GITRL, TIGIT, CD27, VISTA, B7H3, B7H4, HEVM or BTLA (also known as CD272), killer-cell immunoglobulin-like receptors (KIRs), and CD47. Exemplary second antigens include tumor associated antigens (e.g., LINGO1, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, aVB3, a5B1, ERBB2, ERBB3, MET, IGF1R, EPHA3, TRAILR1, TRAILR2, RANKL, FAP and Tenascin), cytokines (e.g., IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL), and cell surface receptors. Different formats of bispecific antibodies are also provided herein. In embodiments, each of the anti-IL-10 or IL-10R fragment and the second fragment is each independently selected from a Fab fragment, a single-chain variable fragment (scFv), or a single-domain antibody. In embodiments, the bispecific antibody further includes a Fc fragment. A bi-specific antibody of the present invention comprises a heavy chain and a light chain combination or scFv of the IL-10 antibodies disclosed herein.

Multispecific antibodies (e.g., bispecific antibodies and trispecific antibodies) of the invention can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker (GGGGSGGGGS; “(G4S)2”) that can be used with scFv fusion constructs described herein can be generated with a longer G4S linker to improve flexibility. For example, the linker can also be “(G4S)3” (e.g., GGGGSGGGGSGGGGS); “(G4S)4” (e.g., GGGGSGGGGSGGGGSGGGGS); “(G4S)5” (e.g., GGGGSGGGGSGGGGSGGGGSGGGGS); “(G4S)6” (e.g., GGGGSGGGGSGGGGSGGGGSGGGGSGGGGS); “(G4S)7” (e.g., GGGGSGGGGSGGGGSGGGGSGGGGSGGGGSGGGGS); and the like. For example, use of the (G4S)5 linker can provide more flexibility and can improve expression. In some embodiments, the linker can also be (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. Non-limiting examples of linkers known to those skilled in the art that can be used are described in U.S. Pat. No. 9,708,412; U.S. Patent Application Publication Nos. US20180134789 and US20200148771; and PCT Publication No. WO2019051122 (each of which are incorporated by reference in their entireties).

In embodiments, the multispecific antibodies (e.g., bispecific antibodies and trispecific antibodies) can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens are mixed to promote heteroligation pairing, which is mediated through the engineered “knob into holes” of the CH3 domains.

In embodiments, multispecific antibodies (e.g., bispecific antibodies and trispecific antibodies) can be constructed through exchange of heavy-light chain dimers from two or more different antibodies to generate a hybrid antibody where the first heavy-light chain dimer recognizes IL-10 and the second heavy-light chain dimer recognizes a second antigen. In some embodiments, the bi-specific antibody can be constructed through exchange of heavy-light chain dimers from two or more different antibodies to generate a hybrid antibody where the first heavy-light chain dimer recognizes a second antigen and the second heavy-light chain dimer recognizes IL-10 or IL-10R. The mechanism for heavy-light chain dimer is similar to the formation of human IgG₄, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG₄ molecules. Heavy chain pairing is also stabilized further by interheavy chain disulfide bridges in the hinge region of the antibody. Specifically, in IgG₄, the hinge region contains the amino acid sequence Cys-Pro-Ser-Cys (in comparison to the stable IgG1 hinge region which contains the sequence Cys-Pro-Pro-Cys) at amino acids 226-230. This sequence difference of Serine at position 229 has been linked to the tendency of IgG₄ to form intrachain disulfides in the hinge region (Van der Neut Kolfschoten, M. et al, 2007, Science 317:1554-1557 and Labrijn, A. F. et al, 2011, Journal of Immunol 187:3238-3246).

Therefore, bi-specific antibodies of the invention can be created through introduction of the R409 residue in the CH3 domain and the Cys-Pro-Ser-Cys sequence in the hinge region of antibodies that recognize IL-10 or IL-10R or a second antigen, so that the heavy-light chain dimers exchange to produce an antibody molecule with one heavy-light chain dimer recognizing IL-10 or IL-10R and the second heavy-light chain dimer recognizing a second antigen, wherein the second antigen is any antigen disclosed herein. Known IgG₄ molecules can also be altered such that the heavy and light chains recognize IL-10 or IL-10R or a second antigen, as disclosed herein. Use of this method for constructing the bi-specific antibodies of the invention can be beneficial due to the intrinsic characteristic of IgG₄ molecules wherein the Fc region differs from other IgG subtypes in that it interacts poorly with effector systems of the immune response, such as complement and Fc receptors expressed by certain white blood cells. This specific property makes these IgG₄-based bi-specific antibodies attractive for therapeutic applications, in which the antibody is required to bind the target(s) and functionally alter the signaling pathways associated with the target(s), however not trigger effector activities.

The bi-specific antibodies disclosed herein can be useful in treatment of medical conditions, for example cancer.

Antibodies of the invention specifically binding an IL-10 or IL-10R protein or fragment thereof can be administered for the prevention or treatment of a cancer in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R._Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

The antibodies (also referred to herein as “agents of the invention” or “active compounds”) and derivatives, fragments, analogs and homologs thereof, can be incorporated into pharmaceutical compositions suitable for administration. Such pharmaceutical compositions can comprise the antibody or agent and a pharmaceutically acceptable carrier. As used herein, the term “pharmaceutically acceptable carrier” can include solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is intended. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Embodiments can include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. For example, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein can refer to physically discrete units suited as unitary dosages for the subject to be treated; each unit containing a predetermined quantity of active compound calculated to produce the therapeutic effect in association with the required pharmaceutical carrier. The specification for the dosage unit forms of the invention are dictated by and directly dependent on the unique characteristics of the active compound and the therapeutic effect to be achieved, and the limitations inherent in the art of compounding such an active compound for the treatment of individuals.

The pharmaceutical compositions can be included in a container, pack, or dispenser together with instructions for administration.

A specific dosage and treatment regimen for a patient will depend upon a variety of factors, such as the antibodies, variant or derivative thereof used, the patient's age, body weight, general health, sex, and diet, and the time of administration, rate of excretion, drug combination, and the severity of the disease being treated. Judgment of such factors by medical caregivers is within the ordinary skill in the art. The amount will also depend on the individual patient to be treated, the route of administration, the type of formulation, the characteristics of the compound used, the severity of the disease, and the effect of interest. The amount used can be determined by pharmacological and pharmacokinetic principles well known in the art.

A therapeutically effective amount of an antibody of the invention can be the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. The dosage administered to a subject (e.g., a patient) of the antigen-binding polypeptides described herein can be 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration can be useful. Further, the dosage and frequency of administration of antibodies of the disclosure can be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where antibody fragments are used, the smallest inhibitory fragment that specifically binds to the binding domain of the target protein is useful. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90:7889-7893 (1993)). The formulation can also contain more than one active compound as necessary for the indication being treated, such as those with complementary activities that do not adversely affect each other. Alternatively, or in addition, the composition can comprise an agent that enhances its function, such as, for example, a cytotoxic agent, cytokine (e.g. IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration can be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid allow the release of molecules for over 100 days, certain hydrogels release proteins for shorter time periods.

Aspects of the invention comprise administering to a subject a nanoparticle. As used herein, the term “nanoparticle” can refer to any particle having a diameter of less than 1000 nanometers (nm). In some embodiments, the nanoparticle has a diameter of less than 300 nm. In some embodiments, the nanoparticles have a diameter of less than 100 nm. In some embodiments, the nanoparticles are micelles, where they comprise encapsulated compartments separated from the bulk solution by a micelle membrane. A “micellar membrane” includes an amphipathic entity assembled to surround and enclose a space or compartment (eg, to limit a lumen). In some embodiments, the nanoparticle comprises an antibody. In some embodiments, the nanoparticle comprises a CD40 agonist antibody. In some embodiments, the nanoparticle comprises an antibody specific for IL-10. In some embodiments, the nanoparticle comprises a vaccine adjuvant. In some embodiments, the nanoparticle comprises monophosphoryl lipid A (MPL). In some embodiments, MPL is inside the nanoparticle. In some embodiments, the CD40 agonist antibody includes, but is not limited to, FGK45, CP-870,984, CP-870,983, APX005M, dacetuzumab, and ChiLob 7/4. In some embodiments, the IL-10 antibody includes, but is not limited to, 49G08_HIL2, 49G08, 48C11, 49H07, 48C06, 53D07, 53H08, 42B02, 43C05, 43F05, 15A02, and 21B08. In some embodiments, the IL-10 or IL-10R antibody is an IL-10 blocking antibody or an IL-10 receptor blocking antibody.

As used herein, the term “nanoparticle composition” can refer to any substance containing at least one nanoparticle. In some embodiments, the nanoparticle composition is a uniform collection of nanoparticles. In some embodiments, the nanoparticle composition is a dispersion or emulsion. In some embodiments, the nanoparticle composition is stable. In some embodiments, the nanoparticle composition includes one or more biologically active agents to be delivered with the nanoparticles. In some embodiments, the nanoparticle compositions are empty nanoparticle compositions (e.g., they do not contain any known therapeutic agent and/or independently active biologically active agent).

Aspects of the invention comprise measuring or detecting biomarkers of a cell proliferative disease, such as cancer, in a biological sample, and thereby measuring response to treatment or disease progression over time. Biomarkers of the invention can be measured in different types of biological samples. Non-limiting examples of biological samples that can be used in methods of the invention, although not intended to be limiting, include stool, plasma, cord blood, neonatal blood, cerebral spinal fluid, tears, vomit, saliva, urine, feces, and meconium. A sample can be prepared to enhance detectability of the biomarkers. For example, a sample from the subject can be fractionated. Any method that enriches for a biomarker polypeptide of interest can be used. Sample preparations, such as prefractionation protocols, are optional and can be necessary to enhance detectability of biomarkers depending on the methods of detection used. For example, sample preparation can be unnecessary if an antibody that specifically binds a biomarker is used to detect the presence of the biomarker in a sample. Sample preparation can involve fractionation of a sample and collection of fractions determined to contain the biomarkers. Methods of prefractionation include, for example, size exclusion chromatography, ion exchange chromatography, heparin chromatography, affinity chromatography, sequential extraction, gel electrophoresis, mass spectrometry, and liquid chromatography.

The methods described herein can involve obtaining a biological sample from the subject. As used herein, the phrase “obtaining a biological sample” can refer to any process for directly or indirectly acquiring a biological sample from a subject. For example, a biological sample can be obtained (e.g., at a point-of-care facility, such as a physician's office, a hospital, laboratory facility) by procuring a tissue or fluid sample (e.g., blood draw, marrow sample, spinal tap) from a subject. Alternatively, a biological sample can be obtained by receiving the biological sample (e.g., at a laboratory facility) from one or more persons who procured the sample directly from the subject. The biological sample can be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

An antibody according to the invention can be used as an agent for detecting the presence of IL-10 or IL-10R (or a protein fragment thereof) in a biological sample. For example, an embodiment can comprise the early detection of cancer relapse or recurrence, prior to radiographic scans. For example, the antibody can contain a detectable label. Antibodies can be polyclonal, monoclonal, or a fragment. An intact antibody, or a fragment thereof (e.g., Fab, scFv, or F_{(ab)2}) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Antibodies directed against an IL-10 or IL-10R protein (or a fragment thereof) can be used in methods known within the art relating to the localization and/or quantitation of an IL-10 or IL-10R protein (e.g., for use in measuring levels of the IL-10 or IL-10R protein within appropriate physiological samples, for use in diagnostic methods, for use in imaging the protein, and the like). In a given embodiment, antibodies specific to an IL-10 protein, or derivative, fragment, analog or homolog thereof, that contain the antibody derived antigen binding domain, are utilized as pharmacologically active compounds (referred to herein as “therapeutics”).

An antibody of the invention specific for an IL-10 or IL-10R protein can be used to isolate an IL-10 or IL-10R polypeptide by standard techniques, such as immunoaffinity, chromatography or immunoprecipitation. Antibodies directed against an IL-10 or IL-10R protein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P or ³H.

As used herein, the terms “treat” or “treatment” can refer to both therapeutic treatment and prophylactic or preventative measures, wherein the object is to prevent or slow down (lessen) an undesired physiological change or disorder, such as the progression of cancer. Beneficial clinical results can include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a cancer, or other cell proliferation-related diseases or disorders. Such diseases or disorders include but are not limited to, e.g., those diseases or disorders associated with aberrant expression of IL-10 or IL-10R. An “IL-10-associated disease or disorder” includes disease states and/or symptoms associated with a disease state, where increased levels of IL-10 and/or activation of cellular signaling pathways involving IL-10 are found. Exemplary IL-10-associated diseases or disorders include, but are not limited to cell-proliferative diseases, such as cancer. For example, cancer includes but is not limited to solid tumor cancers. For example, solid tumors include but are not limited to melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor.

For example, the methods are used to treat, prevent or alleviate a symptom of cancer. In an embodiment, the methods are used to treat, prevent or alleviate a symptom of a solid tumor. Non-limiting examples of other tumors that can be treated by embodiments herein comprise melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor. Additionally, the methods of the invention can be used to treat hematologic cancers such as leukemia and lymphoma. Alternatively, the methods can be used to treat, prevent or alleviate a symptom of a cancer that has metastasized. For example, the cancer can be melanoma, breast cancer, lung cancer, prostate cancer, ovarian cancer, a sarcoma, hepatocellular carcinoma, rectal cancer, pancreatic cancer, liver cancer, biliary tract cancer, or colon cancer.

In embodiments, the invention provides for methods for preventing, treating or alleviating a symptom cancer or a cell proliferative disease or disorder in a subject by administering to the subject a monoclonal antibody, scFv antibody or multi-specific antibody of the invention or a composition comprising the same. For example, the multi-specific antibody can be a bi-specific antibody. For example, an anti-IL-10 or IL-10R antibody can be administered in therapeutically effective amounts.

Antibodies of the invention, including monoclonal, polyclonal, multi-specific, bi-specific, humanized and fully human antibodies, and fragments can be used as therapeutic agents. Such agents will be employed to treat or prevent cancer in a subject. An antibody preparation, for example, one having high specificity and high affinity for its target antigen, is administered to the subject and will have an effect due to its binding with the target. Administration of the antibody can abrogate or inhibit or interfere with an activity of the IL-10 or IL-10R protein.

Subjects at risk for cancer or cell proliferation-related diseases or disorders can include patients who have a family history of cancer or a subject exposed to a known or suspected cancer-causing agent. Administration of a prophylactic agent can occur prior to the manifestation of cancer such that the disease is prevented or, alternatively, delayed in its progression.

The term “subject” or “patient” can refer to any organism to which aspects of the invention can be administered, e.g., for experimental, diagnostic, prophylactic, research and/or therapeutic purposes. For example, subjects to which compounds of the disclosure can be administered will be mammals, such as primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals for example, pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

In embodiments, a subject comprises a mammal, such as a human or vertebrate animal. Examples of such include but are not limited to a dog, cat, horse, cow, pig, sheep, goat, chicken, primate, e.g., monkey, fish (aquaculture species), e.g. salmon, rat, and mouse. A human comprises a preterm neonate, an infant, a child, an adolescent, an adult, or an elderly individual.

Although aspects of the invention as described herein relate to human cell proliferative disorders, aspects of the invention are also applicable to other nonhuman vertebrates. Aspects of the invention are applicable for veterinary use, such as with domestic animals. Aspects will vary according to the type of use and mode of administration, as well as the particularized requirements of individual subjects.

In another aspect, tumor cell growth is inhibited by contacting a cell with an anti-IL-10 antibody of the invention. The cell can be any cell that expresses IL-10 or IL-10R.

Aspects of the invention can be used in methods of in situ vaccination (ISV). In situ vaccination (ISV) triggers negative feedback mechanisms involving the secretion of inhibitory cytokines (e.g., TGFβ, IL10) that suppress antigen presenting cell (APC) activation and APCs' ability to trigger an immune response. Yet ISV that includes blockade of these pathways, as a component of the in situ vaccine itself, has not been previously studied. The local secretion of IL10 in response to ISV represents a critical barrier to optimal efficacy. Here, embodiments comprise in situ vaccines and methods that include blockade of the IL10-IL10 receptor (αIL10R) axis using anti-IL-10 and/or anti-IL-10R antibodies to overcome this barrier.

Compositions of the invention as described herein can be administered in combination with one or more additional active agents. For example, the additional active agent can be a chemotherapeutic agent, an immunotherapeutic agent, a radiotherapeutic agent, or a cytokine.

Non-limiting examples of a chemotherapeutic agent that can be administered with the compositions described herein include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide). In addition, the antibody can be combined with targeted agents such as IL-10 and other receptor tyrosine kinase inhibitors, MMP-9 inhibitors, epigenetic agents and immunotherapy agents such as checkpoint inhibitors.

In embodiments, the compositions of the invention as described herein can be administered in combination with cytokines. Cytokines that can be administered with the compositions include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions described herein can be administered in combination with other therapeutic or prophylactic regimens, such as, for example, radiation therapy.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

The invention provides for methods of treating cancer in a patient by administering two antibodies that bind to the same epitope of the IL-10 or IL-10R protein or, alternatively, two different epitopes of the IL-10 or IL-10R protein. Alternatively, the cancer can be treated by administering a first antibody that binds to IL-10 or IL-10R and a second antibody that binds to a protein other than IL-10 or IL-10R. In other embodiments, the cancer can be treated by administering a bispecific antibody that binds to IL-10 or IL-10R and that binds to a protein other than IL-10 or IL-10R. For example, the other protein other than IL-10 or IL-10R can include, but is not limited to, targets as described herein. For example, the other protein other than IL-10 or IL-10R can be a tumor-associated antigen; the other protein other than IL-10 or IL-10R can also be a cytokine.

In embodiments, the invention provides for the administration of an anti-IL-10 or anti-IL-10R antibody alone or in combination with an additional antibody that recognizes another protein other than IL-10 or IL-10R, with cells that can effect or augment an immune response. For example, these cells can be peripheral blood mononuclear cells (PBMC), or any cell type that is found in PBMC, e.g., cytotoxic T cells, macrophages, and natural killer (NK) cells.

The invention provides administration of an antibody that binds to the IL-10 protein and an anti-neoplastic agent, such a small molecule, a growth factor, a cytokine, or other therapeutics including biomolecules such as peptides, peptidomimetics, peptoids, polynucleotides, lipid-derived mediators, small biogenic amines, hormones, neuropeptides, and proteases. Small molecules include, but are not limited to, inorganic molecules and small organic molecules. Suitable growth factors or cytokines include an IL-2, GM-CSF, IL-12, and TNF-alpha. Small molecule libraries are known in the art. (See, Lam, Anticancer Drug Des., 12:145, 1997)

Cellular therapies, such as chimeric antigen receptor (CAR) T-cell therapies, are also provided herein. CAR T-cell therapies redirect a patient's T-cells to kill tumor cells by the exogenous expression of a CAR. A CAR can be a membrane spanning fusion protein that links the antigen recognition domain of an antibody to the intracellular signaling domains of the T-cell receptor and co-receptor. A suitable cell can be used, that is put in contact with an anti-IL-10 or IL-10R antibody of the invention (or alternatively engineered to express an anti-IL-10 or IL-10R antibody as described herein). Solid tumors offer unique challenges for CAR-T therapies. Unlike blood cancers, tumor-associated target proteins are overexpressed between the tumor and healthy tissue resulting in on-target/off-tumor T-cell killing of healthy tissues. Furthermore, immune repression in the tumor microenvironment (TME) limits the activation of CAR-T cells towards killing the tumor. Upon such contact or engineering, the cell can then be introduced to a cancer patient in need of a treatment. The cancer patient can have a cancer of any of the types as disclosed herein. The cell (e.g., a T cell) can be, for instance, a tumor-infiltrating T lymphocyte, a CD4+ T cell, a CD8+ T cell, or the combination thereof, without limitation.

For example, embodiments of the invention comprise chimeric antigen receptor (CAR) comprising an intracellular signaling domain, a transmembrane domain and an extracellular domain. In embodiments, the extracellular domain is an isolated monoclonal antibody or antigen-binding fragment thereof that binds to human Interleukin 10 (IL-10) protein or IL-10R protein. For example, the monoclonal antibody or fragment thereof comprises a heavy chain, light chain, or combination thereof, wherein the heavy chain comprises a CDR1, CDR2, and/or CDR3 according to Tables 13A-24A; and wherein the light chain comprises a CDR1, CDR2, and/or CDR3 according to Tables 13B-24B.

The CAR according to the invention can comprise at least one transmembrane polypeptide comprising at least one extracellular ligand-biding domain and; one transmembrane polypeptide comprising at least one intracellular signaling domain; such that the polypeptides assemble together to form a Chimeric Antigen Receptor.

The term “extracellular ligand-binding domain” as used herein can refer to an oligo- or polypeptide that binds a ligand. For example, the domain can interact with a cell surface molecule. For example, the extracellular ligand-binding domain can be chosen to recognize a ligand that acts as a cell surface marker on target cells associated with a disease state.

In an embodiment, the extracellular ligand-binding domain can comprise an antigen binding domain derived from an antibody against an antigen of the target. For example, the target can be IL-10. Thus, the CAR can be specific for IL-10 or IL-10R. In an embodiment, said extracellular ligand-binding domain is a single chain antibody fragment (scFv) comprising the light (VL) and the heavy (VH) variable fragment of a target antigen specific monoclonal antibody joined by a flexible linker. For example, said scFv antibody is specific for IL-10 or IL-10R. It is understood, however, that binding domains other than scFv can also be used for predefined targeting of lymphocytes, such as camelid single-domain antibody fragments or receptor ligands, antibody binding domains, antibody hypervariable loops or CDRs as non limiting examples.

In embodiments said transmembrane domain comprises a stalk region between said extracellular ligand-binding domain and said transmembrane domain. The term “stalk region” can refer to any oligo- or polypeptide that functions to link the transmembrane domain to the extracellular ligand-binding domain. For example, stalk region(s) is/are used to provide more flexibility and accessibility for the extracellular ligand-binding domain. A stalk region can comprise up to 300 amino acids, such as 10 to 100 amino acids. In embodiments, the stalk region comprises 25 to 50 amino acids. Stalk region can be derived from naturally occurring molecules, such as from the extracellular region of CD8, CD4 or CD28, or from an antibody constant region. Alternatively, the stalk region can be a synthetic sequence that corresponds to a naturally occurring stalk sequence, or can be an entirely synthetic stalk sequence. In an embodiment said stalk region is a part of human CD8 alpha chain.

In embodiments, the transmembrane domain can comprise CD28.

The signal transducing domain or intracellular signaling domain of the CAR of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” can refer to the portion of a protein which transduces the effector signal function signal and directs the cell to perform a specialized function.

Signal transduction domain can comprise two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs (immunoreceptor tyrosine-based activation motifs). ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCR zeta, FcR gamma, FcR beta, FcR epsilon, CD3 gamma, CD3 delta, CD3 epsilon, CD5, CD22, CD79a, CD79b and CD66d. In some embodiments, the signaling transducing domain of the CAR can comprise the CD3 zeta signaling domain, or the intracytoplasmic domain of the Fc epsilon R1 beta or gamma chains. In another embodiment, the signaling is provided by CD3 zeta together with co-stimulation provided by CD28 and a tumor necrosis factor receptor (TNFr), such as 4-1BB or OX40), for example.

In embodiments, the intracellular signaling domain of the CAR of the invention comprises a co-stimulatory signal molecule. In embodiments the intracellular signaling domain contains 2, 3, 4 or more co-stimulatory molecules in tandem. A co-stimulatory molecule is a cell surface molecule other than an antigen receptor or their ligands that is required for an efficient immune response.

“Co-stimulatory ligand” can refer to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, MICB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LIGHT, NKG2C, B7-H3, a ligand that specifically binds with CD83.

A “co-stimulatory molecule” can refer to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class 1 molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In embodiments, the choice of CD28 as a co-stimulatory domain for the CARs can be based in the fact that CD28 CARs direct an active proliferative response and enhance effector functions, whereas 4-1BB-based CARs induce a more progressive T cell accumulation that can counterweigh for less immediate effectiveness. In one embodiment, the CD28 is replaced by 41BB in the CAR constructs.

In an embodiment, said signal transducing domain is a TNFR-associated Factor 2 (TRAF2) binding motifs, intracytoplasmic tail of costimulatory TNFR member family. Cytoplasmic tail of costimulatory TNFR family member contains TRAF2 binding motifs consisting of the major conserved motif (P/S/A)X(Q/E)E) or the minor motif (PXQXXD), wherein X is any amino acid. TRAF proteins are recruited to the intracellular tails of many TNFRs in response to receptor trimerization.

The distinguishing features of appropriate transmembrane polypeptides comprise the ability to be expressed at the surface of an immune cell, such as lymphocyte cells or Natural killer (NK) cells, and to interact together for directing cellular response of immune cell against a predefined target cell. The different transmembrane polypeptides of the CAR of the invention comprising an extracellular ligand-biding domain and/or a signal transducing domain interact together to take part in signal transduction following the binding with a target ligand and induce an immune response. The transmembrane domain can be derived from a natural or from a synthetic source. The transmembrane domain can be derived from any membrane-bound or transmembrane protein.

The term “a part of” can refer to any subset of the molecule, that is a shorter peptide. Alternatively, amino acid sequence functional variants of the polypeptide can be prepared by mutations in the DNA which encodes the polypeptide. Such variants or functional variants include, for example, deletions from, or insertions or substitutions of, residues within the amino acid sequence. Any combination of deletion, insertion, and substitution can also be made to arrive at the final construct, provided that the final construct possesses the targeted activity, especially to exhibit a specific anti-target cellular immune activity. The functionality of the CAR of the invention within a host cell is detectable in an assay suitable for demonstrating the signaling potential of said CAR upon binding of a target. Such assays are available to the skilled person in the art. For example, this assay allows the detection of a signaling pathway, triggered upon binding of the target, such as an assay involving measurement of the increase of calcium ion release, intracellular tyrosine phosphorylation, inositol phosphate turnover, or interleukin (IL) 2, interferon γ, GM-CSF, IL-3, IL-4 production thus effected.

Embodiments of the invention include cells that express a CAR (i.e, CARTS). The cell can be of any kind, including an immune cell expressing the CAR for cancer therapy or a cell, such as a bacterial cell, that harbors an expression vector that encodes the CAR. As used herein, the terms “cell,” “cell line,” and “cell culture” can be used interchangeably. These terms also include their progeny, which includes subsequent generations. For example, progeny does not need to be identical, such as due to deliberate or inadvertent mutations. In the context of expressing a heterologous nucleic acid sequence, “host cell” can refer to a eukaryotic cell that can replicate a vector and/or expressing a heterologous gene encoded by a vector. A host cell can, and has been, used as a recipient for vectors. A host cell can be “transfected” or “transformed,” which can refer to a process by which exogenous nucleic acid is transferred or introduced into the host cell. A transformed cell includes the primary subject cell and its progeny. As used herein, the terms “engineered” and “recombinant” cells or host cells can refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T cell (also known as TC, Cytotoxic T Lymphocyte, CTL, T-Killer cell, cytolytic T cell, CD8+ T-cells or killer T cell); NK cells and NKT cells are also encompassed in the invention.

Vectors can employ control sequences that allow it to be replicated and/or expressed in both prokaryotic and eukaryotic cells. One of skill in the art can understand the conditions under which to incubate the host cells described herein to maintain them and to permit replication of a vector. Also understood and known are techniques and conditions that can allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one can wish to be able to kill the modified CTLs, where one wishes to terminate the treatment, the cells become neoplastic, in research where the absence of the cells after their presence is of interest, or other event. For this purpose, one can provide for the expression of certain gene products in which one can kill the modified cells under controlled conditions, such as inducible suicide genes.

Expression vectors that encode the CARs can be introduced as one or more DNA molecules or constructs, where there can be at least one marker that will allow for selection of host cells that contain the construct(s).

The constructs can be prepared in conventional ways, where the genes and regulatory regions can be isolated, as appropriate, ligated, cloned in an appropriate cloning host, analyzed by restriction or sequencing, or other convenient means. Using PCR, individual fragments including a functional unit can be isolated, where one or more mutations can be introduced using “primer repair”, ligation, in vitro mutagenesis, etc., as appropriate. The construct(s) once completed and demonstrated to have the appropriate sequences can then be introduced into the CTL by any convenient means. The constructs can be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs can include viral sequences for transfection. Alternatively, the construct can be introduced by fusion, electroporation, biolistics, transfection, lipofection, or the like. The host cells can be grown and expanded in culture before introduction of the construct(s), followed by the appropriate treatment for introduction of the construct(s) and integration of the construct(s). The cells are then expanded and screened by virtue of a marker present in the construct. Various markers that can be used successfully include hprt, neomycin resistance, thymidine kinase, hygromycin resistance, etc.

In embodiments, one can have a target site for homologous recombination, where a construct is to be integrated at a certain locus. For example, one can knock-out an endogenous gene and replace it (at the same locus or elsewhere) with the gene encoded for by the construct using materials and methods as are known in the art for homologous recombination. For homologous recombination, one can use either OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs can be introduced as a single DNA molecule encoding at least the CAR and optionally another gene, or different DNA molecules having one or more genes. Other genes include genes that encode therapeutic molecules or suicide genes, for example. The constructs can be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that can be used to prepare stocks of construct DNAs and for carrying out transfections are well known in the art, and many are commercially available.

The cells described herein can be used for treating a cancer, or other cell proliferation-related diseases or disorders. Such diseases or disorders include but are not limited to, e.g., those diseases or disorders associated with aberrant expression of IL-10 or IL-10R. In embodiments, said isolated cell according to the invention can be used in the manufacture of a medicament for treatment a cancer, or other cell proliferation-related diseases or disorders. Such diseases or disorders include but are not limited to, e.g., those diseases or disorders associated with aberrant expression of IL-10 or IL-10R.

Embodiments described herein rely on methods for treating patients in need thereof, said method comprising at least one of the following steps: (a) providing a chimeric antigen receptor cells according to the invention and (b) administrating the cells to said patient.

Said treatment can be ameliorating, curative or prophylactic. It can be part of an autologous immunotherapy or part of an allogenic immunotherapy treatment. For example, autologous can refer to cells, a cell line or population of cells used for treating patients that originate from said patient or from a Human Leucocyte Antigen (HLA) compatible donor. For example, allogeneic can refer to cells or population of cells used for treating patients that do not originate from said patient but from a donor.

The invention is suited for allogenic immunotherapy, insofar as it allows the transformation of T-cells, for example, obtained from donors, into non-alloreactive cells. This can be done under standard protocols and reproduced as many times as needed. The resulted modified T cells can be pooled and administrated to one or several patients, being made available as an “off the shelf” therapeutic product.

For example, treatment can be antibody and/or CAR-T treatment in combination with one or more therapies against cancer selected from the group of antibodies therapy, chemotherapy, cytokines therapy, dendritic cell therapy, gene therapy, hormone therapy, laser light therapy and radiation therapy.

According to an embodiment of the invention, said treatment can be administrated into patients undergoing an immunosuppressive treatment. Indeed, the invention can rely on cells or population of cells, which have been made resistant to at least one immunosuppressive agent due to the inactivation of a gene encoding a receptor for such immunosuppressive agent. In aspects, the immunosuppressive treatment can help the selection and expansion of the T-cells according to the invention within the patient.

In an embodiment, the cell compositions of the invention are administered to a patient in conjunction with (e.g., before, simultaneously or following) bone marrow transplantation, T cell ablative therapy using chemotherapy agents such as, fludarabine, external-beam radiation therapy (XRT), cyclophosphamide, or antibodies such as OKT3 or CAM PATH. In another embodiment, the cell compositions of the invention are administered following B-cell ablative therapy such as agents that react with CD20, e.g., Rituxan. For example, in one embodiment, subjects can undergo standard treatment with high dose chemotherapy followed by peripheral blood stem cell transplantation. In certain embodiments, following the transplant, subjects receive an infusion of the expanded immune cells of the invention. In an additional embodiment, expanded cells are administered before or following surgery. Said modified cells obtained by any one of the methods described herein can be used in an aspect of the invention for treating patients in need thereof against Host versus Graft (HvG) rejection and Graft versus Host Disease (GvHD); therefore in the scope of the invention is a method of treating patients in need thereof against Host versus Graft (HvG) rejection and Graft versus Host Disease (GvHD) comprising treating said patient by administering to said patient an effective amount of modified cells comprising inactivated TCR alpha and/or TCR beta genes.

The invention is suited for allogenic immunotherapy, insofar as it allows the transformation of T-cells, for example, obtained from donors, into non-alloreactive cells. This can be done under standard protocols and reproduced as many times as needed. The resulted modified T cells can be pooled and administrated to one or several patients, being made available as an “off the shelf” therapeutic product.

Depending upon the nature of the cells, the cells can be introduced into a host organism, e.g. a mammal, in a wide variety of ways. The cells can be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells can be applied as a dispersion, for example being injected at or near the site of interest. The cells can be in a physiologically acceptable medium.

In embodiments, the cells are encapsulated to inhibit immune recognition and placed at the site of the tumor.

The cells can be administered as desired. Depending upon the response of interest, the manner of administration, the life of the cells, the number of cells present, various protocols can be employed. The number of administrations will depend upon the factors described herein at least in part.

The administration of the cells or population of cells according to the invention can be carried out in any convenient manner, including by aerosol inhalation, injection, ingestion, transfusion, implantation or transplantation. The compositions described herein can be administered to a patient subcutaneously, intradermally, intratumorally, intranodally, intramedullary, intramuscularly, by intravenous or intralymphatic injection, or intraperitoneally. In an embodiment, the cell compositions of the invention are administered by intravenous injection.

The administration of the cells or population of cells can consist of the administration of 10⁴ to 10⁹ cells per kg body weight, such as 10⁵ to 10⁶ cells/kg body weight including integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells can be obtained from any source, such as a blood bank or a donor. While individual needs vary, determination of optimal ranges of effective amounts of a given cell type for a disease or conditions within the skill of the art. An effective amount can refer to an amount which provides a therapeutic or prophylactic benefit. The dosage administrated will be dependent upon the age, health and weight of the recipient, kind of concurrent treatment, if any, frequency of treatment and the nature of the effect of interest.

It can be appreciated that the system is subject to many variables, such as the cellular response to the ligand, the efficiency of expression and, as appropriate, the level of secretion, the activity of the expression product, the need of the patient, which can vary with time and circumstances, the rate of loss of the cellular activity as a result of loss of cells or expression activity of individual cells, and the like. Therefore, for each individual patient, even if there were universal cells which can be administered to the population at large, each patient can be monitored for the proper dosage for the individual, and such practices of monitoring a patient are routine in the art.

Monoclonal antibodies and CARs of the invention can be expressed from an expression vector. Recombinant techniques to generate such expression vectors are well known in the art.

The term “vector” can refer to a carrier nucleic acid molecule into which a nucleic acid sequence can be inserted for introduction into a cell where it can be replicated. A nucleic acid sequence can be “exogenous,” for example, that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art can be well equipped to construct a vector through standard recombinant techniques (see, for example, Maniatis et al., 1988 and Ausubel et al., 1994, both incorporated herein by reference).

The term “expression vector” can refer to any type of genetic construct comprising a nucleic acid coding for an RNA to be transcribed. In cases, RNA molecules are then translated into a protein, polypeptide, or peptide. In other cases, these sequences are not translated, for example, in the production of antisense molecules or ribozymes. Expression vectors can contain a variety of “control sequences,” which can refer to nucleic acid sequences necessary for the transcription and without wishing to be bound by theory, translation of an operably linked coding sequence in a host cell. In addition to control sequences that govern transcription and translation, vectors and expression vectors can contain nucleic acid sequences that serve other functions as well and are described herein.

A “promoter” can refer to a control sequence that is a region of a nucleic acid sequence at which initiation and rate of transcription are controlled. It can contain genetic elements at which regulatory proteins and molecules can bind, such as RNA polymerase and other transcription factors, to initiate the specific transcription a nucleic acid sequence. The phrases “operatively positioned,” “operatively linked,” “under control,” and “under transcriptional control” can refer to a promoter is in a correct functional location and/or orientation in relation to a nucleic acid sequence to control transcriptional initiation and/or expression of that sequence.

A promoter can comprise a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. These can be located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function cooperatively or independently to activate transcription. A promoter can be used in conjunction with an “enhancer,” which can refer to a cis-acting regulatory sequence involved in the transcriptional activation of a nucleic acid sequence.

A promoter can be one naturally associated with a nucleic acid sequence, as can be obtained by isolating the 5 prime′ non-coding sequences located upstream of the coding segment and/or exon. Such a promoter can be referred to as “endogenous.” Similarly, an enhancer can be one naturally associated with a nucleic acid sequence, located downstream or upstream of that sequence. Alternatively, advantages will be gained by positioning the coding nucleic acid segment under the control of a recombinant or heterologous promoter, which can refer to a promoter that is not normally associated with a nucleic acid sequence in its natural environment. A recombinant or heterologous enhancer can also refer to an enhancer not normally associated with a nucleic acid sequence in its natural environment. Such promoters or enhancers can include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are used in recombinant DNA construction include the lactamase (penicillinase), lactose and tryptophan (trp) promoter systems. In addition to producing nucleic acid sequences of promoters and enhancers synthetically, sequences can be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the organelle, cell type, tissue, organ, or organism chosen for expression. Those of skill in the art of molecular biology know the use of promoters, enhancers, and cell type combinations for protein expression, (see, for example Sambrook et al. 1989, incorporated herein by reference). The promoters employed can be constitutive, tissue-specific, inducible, and/or useful under the appropriate conditions to direct high level expression of the introduced DNA segment, such as is advantageous in the large-scale production of recombinant proteins and/or peptides. The promoter can be heterologous or endogenous.

Additionally, any promoter/enhancer combination can also be used to drive expression. Use of a T3, T7 or SP6 cytoplasmic expression system is another embodiment. Eukaryotic cells can support cytoplasmic transcription from certain bacterial promoters if the appropriate bacterial polymerase is provided, as part of the delivery complex or as an additional genetic expression construct.

The identity of tissue-specific promoters or elements, as well as assays to characterize their activity, is well known to those of skill in the art.

A specific initiation signal also can be required for efficient translation of coding sequences. These signals include the ATG initiation codon or adjacent sequences. Exogenous translational control signals, including the ATG initiation codon, may need to be provided. One of ordinary skill in the art can determine this and provide the necessary signals.

In embodiments of the invention, the use of internal ribosome entry sites (IRES) elements are used to create multigene, or polycistronic, messages, and these can be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” can refer to catalytic cleavage of a nucleic acid molecule with an enzyme that functions only at specific locations in a nucleic acid molecule. Many of these restriction enzymes are commercially available. Use of such enzymes is widely understood by those of skill in the art. A vector can be linearized or fragmented using a restriction enzyme that cuts within the MCS to allow exogenous sequences to be ligated to the vector. “Ligation” can refer to the process of forming phosphodiester bonds between two nucleic acid fragments, which can be contiguous with each other. Techniques involving restriction enzymes and ligation reactions are well known to those of skill in the art of recombinant technology.

Splicing sites, termination signals, origins of replication, and selectable markers can also be employed.

In embodiments, a plasmid vector can be used to transform a host cell. Plasmid vectors containing replicon and control sequences which are derived from species compatible with the host cell can be used in connection with these hosts. The vector ordinarily carries a replication site, as well as marking sequences that provide phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™ 11 can be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors can be induced, as can be understood by those of skill in the art, by contacting a host cell with an agent specific for certain promoters, e.g., by adding IPTG to the media or by switching incubation to a higher temperature. After culturing the bacteria for a further period, such as of between 2 and 24 h, the cells are collected by centrifugation and washed to remove residual media.

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the invention can be a viral vector that encodes one or more monoclonal antibodies or CARs of the invention. Non-limiting examples of virus vectors that can be used to deliver a nucleic acid of the invention are described herein.

A method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” can include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

The nucleic acid can be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the invention as it has a high frequency of integration and it can infect nondividing cells, thus making it useful for delivery of genes into mammalian cells, for example, in tissue culture (Muzyczka. 1992) or in vivo. AAV has a broad host range for infectivity (Tratschin et al., 1984; Laughlin et al., 1986; Lebkowski et al., 1988; Mclaughlin et al., 1988). Details for the generation and use of rAAV vectors are described in U.S. Pat. Nos. 5,139,941 and 4,797,368, each incorporated herein by reference.

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the targeted sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors can infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al . . . 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors can infect non-dividing cells and can be used for both in vivo and ex vivo gene transfer and expression of nucleic acid sequences. For example, recombinant lentivirus can infect a non-dividing cell wherein a suitable host cell is transfected with two or more vectors carrying the packaging functions, namely gag, pol and env, as well as rev and tat is described in U.S. Pat. No. 5,994,136, incorporated herein by reference. One can target the recombinant virus by linkage of the envelope protein with an antibody or a ligand for targeting to a receptor of a cell-type. By inserting a sequence (including a regulatory region) of interest into the viral vector, along with another gene which encodes the ligand for a receptor on a specific target cell, for example, the vector is now target-specific.

Other viral vectors can be employed as vaccine constructs in the invention. Vectors derived from viruses such as vaccinia virus (Ridgeway, 1988; Baichwal and Sugden, 1986; Coupar et al., 1988), sindbis virus, cytomegalovirus and herpes simplex virus can be employed. They offer several attractive features for various mammalian cells (Friedmann, 1989; Ridgeway, 1988; Baichwal and Sugden, 1986; Coupar et al., 1988; Horwich et al., 1990).

In embodiments, a nucleic acid to be delivered can be housed within an infective virus that has been engineered to express a specific binding ligand. The virus particle will thus bind specifically to the cognate receptors of the target cell and deliver the contents to the cell. An approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

Methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells can be stably or transiently transformed.

Methods for transfecting eukaryotic cells and tissues removed from an organism in an ex vivo setting are known to those of skill in the art. Thus, cells or tissues can be removed and transfected ex vivo using nucleic acids of the invention. In aspects, the transplanted cells or tissues can be placed into an organism. In embodiments, a nucleic acid is expressed in the transplanted cells.

Any of the compositions described herein can be comprised in a kit.

Some components of the kits can be packaged in aqueous media or in lyophilized form. The container means of the kits can include at least one vial, test tube, flask, bottle, syringe or other container means, into which a component can be placed, and suitably aliquoted. Where there is more than one component in the kit, the kit also can contain a second, third or other additional container into which the additional components can be separately placed. However, various combinations of components can be comprised in a vial. The kits of the invention also can include a means for containing the components in close confinement for commercial sale. Such containers can include injection or blow molded plastic containers into which the vials are retained.

When the components of the kit are provided in one and/or more liquid solutions, the liquid solution is an aqueous solution, with a sterile aqueous solution being useful. In some cases, the container means can itself be a syringe, pipette, and/or other such like apparatus, from which the formulation can be applied to an infected area of the body, injected into an animal, and/or even applied to and/or mixed with the other components of the kit.

However, the components of the kit can be provided as dried powder(s). When reagents and/or components are provided as a dry powder, the powder can be reconstituted by the addition of a suitable solvent. Without wishing to be bound by theory, the solvent can also be provided in another container means. The kits can also comprise a second container means for containing a sterile, pharmaceutically acceptable buffer and/or another diluent.

In embodiments of the invention, antibodies and/or cells that are to be used for therapy are provided in a kit, and in some cases the antibodies and/or cells can be the sole component of the kit. The kit can comprise reagents and materials to make the antibody and/or cell of interest. In embodiments, the reagents and materials include primers for amplifying sequences, nucleotides, suitable buffers or buffer reagents, salt, and so forth, and in some cases the reagents include vectors and/or DNA that encodes for antibodies or a CAR as described herein and/or regulatory elements therefor.

In embodiments, there are one or more apparatuses in the kit suitable for extracting one or more samples from an individual. The apparatus can be a syringe, scalpel, and so forth.

In embodiments of the invention, the kit, in addition to cell therapy embodiments, also includes a second cancer therapy, such as chemotherapy, hormone therapy, and/or immunotherapy, for example. The kit(s) can be tailored to a cancer for an individual and comprise respective second cancer therapies for the individual.

Aspects of the invention comprise assays, for example assays that detect the presence of and/or measure levels of IL-10 or a fragment thereof.

Embodiments of the invention comprise measuring or detecting IL-10 using assays known to the art. Non-limiting examples of assays include an immunoassay, a colorimetric assay, fluorimetric assay or a combination thereof. Non-limiting examples of immunoassays comprise a western blot assay, an enzyme-linked immunosorbent assay (ELISA), immunoprecipitation or a combination thereof. For example, a biological sample collected from a subject can be incubated together with an anti-IL-10 or IL-10R antibody according to the invention, and the binding of the antibody to the biomarker in the sample is detected or measured. In embodiments, the antibody or fragment thereof can be specific for IL-10 or IL-10R. The antibody can be a polyclonal antibody or a monoclonal antibody. The antibody or fragment thereof can be attached to a molecule for use in identification, visualization, or localization using known methods. Detectable labels include but are not limited to radioisotopic labels, enzyme labels, non-radioactive isotopic labels, fluorescent labels, toxin labels, affinity labels, and chemiluminescent labels.

In embodiments, the assays can be provided in a multi-well format, such as a 6-, 12-, 24-, 48, or 96-well plate.

The anti-IL-10 or anti-IL-10R antibodies can be used diagnostically to, for example, detect cancer or a cell-proliferative disease, detect the recurrence of cancer or a cell-proliferative disease, monitor the development or progression of cancer as part of a clinical testing procedure to, e.g., determine the efficacy of a given treatment and/or prevention regimen.

“Changed as compared to a control” sample or subject is understood as having a level of the analyte or diagnostic or therapeutic indicator (e.g., marker such as IL-10) to be detected at a level that is statistically different than a sample from a normal, untreated, or abnormal state control sample. Determination of statistical significance is within the ability of those skilled in the art, e.g., the number of standard deviations from the mean that constitute a positive or negative result and the statistical analyses to arrive at these intervals.

If a subject is diagnosed with a cell-proliferative disease, such as cancer, embodiments of the invention comprise treating the subject. For example, treating the subject can comprise administering to the subject an effective anti-cancer agent, including those described herein.

The term “threshold”, for example, a threshold indicative of a cell-proliferative disease, such as cancer, can refer to a value derived from a plurality of biological samples, such as cancer samples or donor blood samples, for a biomarker, such as IL-10 protein levels, above which threshold is associated with an increased likelihood of having and/or developing a cell proliferative disease, such as cancer.

In aspects for diagnostic purposes, the anti-IL-10 antibody of the invention can be linked to a detectable moiety, for example, so as to provide a method for detecting a cancer cell in a subject at risk of or suffering from a cancer.

The detectable moieties can be conjugated directly to the antibodies or fragments, or indirectly by using, for example, a fluorescent secondary antibody. Direct conjugation can be accomplished by standard chemical coupling of, for example, a fluorophore to the antibody or antibody fragment, or through genetic engineering. Chimeras, or fusion proteins can be constructed which contain an antibody or antibody fragment coupled to a fluorescent or bioluminescent protein. For example, Casadei, et al. (Proc Natl Acad Sci USA. 1990 March; 87 (6): 2047-51) describe a method of making a vector construct that can express a fusion protein of aequorin and an antibody gene in mammalian cells.

The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject (such as a biopsy), as well as tissues, cells and fluids present within a subject. That is, the detection method of the invention can be used to detect cells that express IL-10 in a biological sample in vitro as well as in vivo. For example, in vitro techniques for detection of IL-10 include enzyme linked immunosorbent assays (ELISAs), Western blots, immunoprecipitations, and immunofluorescence. Furthermore, in vivo techniques for detection of IL-10 or IL-10R include introducing into a subject a labeled anti-IL-10 or anti-IL-10R antibody. For example, the antibody can be labeled with a radioactive marker whose presence and location in a subject can be detected by standard imaging techniques.

In the case of “targeted” conjugates, that is, conjugates which contain a targeting moiety—a molecule or feature designed to localize the conjugate within a subject or animal at a site or sites, localization can refer to a state when an equilibrium between bound, “localized”, and unbound, “free” entities within a subject has been essentially achieved. The rate at which such equilibrium is achieved depends upon the route of administration. For example, a conjugate administered by intravenous injection can achieve localization within minutes of injection. On the other hand, a conjugate administered orally can take hours to achieve localization. Alternatively, localization can refer to the location of the entity within the subject or animal at selected time periods after the entity is administered. By way of another example, localization is achieved when a moiety becomes distributed following administration.

A reasonable estimate of the time to achieve localization can be made by one skilled in the art. Furthermore, the state of localization as a function of time can be followed by imaging the detectable moiety (e.g., a light-emitting conjugate) according to the methods of the invention, such as with a photodetector device. The “photodetector device” used can have a high enough sensitivity to allow the imaging of faint light from within a mammal in a reasonable amount of time, and to use the signal from such a device to construct an image.

In cases where light-generating moieties can be used which are extremely bright, and/or to detect light-generating fusion proteins localized near the surface of the subject or animal being imaged, a pair of “night-vision” goggles or a standard high-sensitivity video camera, such as a Silicon Intensified Tube (SIT) camera (e.g., from_Hammamatsu Photonic Systems, Bridgewater, N.J.), can be used. However, a more sensitive method of light detection is required.

In extremely low light levels, the photon flux per unit area becomes so low that the scene being imaged no longer appears continuous. Instead, it is represented by individual photons which are both temporally and spatially distinct form one another. Viewed on a monitor, such an image appears as scintillating points of light, each representing a single detected photon. By accumulating these detected photons in a digital image processor over time, an image can be acquired and constructed. In contrast to conventional cameras where the signal at each image point is assigned an intensity value, in photon counting imaging the amplitude of the signal carries no significance. The objective is to detect the presence of a signal (photon) and to count the occurrence of the signal with respect to its position over time.

At least two types of photodetector devices, described herein, can detect individual photons and generate a signal which can be analyzed by an image processor. Reduced-Noise Photodetection devices achieve sensitivity by reducing the background noise in the photon detector, as opposed to amplifying the photon signal. Noise is reduced primarily by cooling the detector array. The devices include charge coupled device (CCD) cameras that can be to as “backthinned”, cooled CCD cameras. In the more sensitive instruments, the cooling is achieved using, for example, liquid nitrogen, which brings the temperature of the CCD array to approximately −120° C. “Backthinned” can refer to an ultra-thin backplate that reduces the path length that a photon follows to be detected, thereby increasing the quantum efficiency. A sensitive backthinned cryogenic CCD camera is the “TECH 512”, a series 200 camera available from Photometries, Ltd. (Tucson, Ariz.).

“Photon amplification devices” amplify photons before they hit the detection screen. This class includes CCD cameras with intensifiers, such as microchannel intensifiers. A microchannel intensifier can contain a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image processors process signals generated by photodetector devices which count photons in order to construct an image which can be, for example, displayed on a monitor or printed on a video printer. Such image processors are sold as part of systems which include the sensitive photon-counting cameras described herein, and accordingly, are available from the same sources. The image processors can be connected to a personal computer, such as an IBM-compatible PC or an Apple Macintosh (Apple Computer, Cupertino, Calif), which can be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

In an embodiment, the biological sample contains protein molecules from the test subject. For example, a biological sample can be a peripheral blood leukocyte sample isolated by conventional means from a subject.

The invention also encompasses kits for detecting the presence of IL-10 or an IL-10-expressing cell in a biological sample. For example, the kit can comprise: a labeled compound or agent that can detect a cancer or tumor cell (e.g., an anti-IL-10 or anti-IL-10R monoclonal antibody) in a biological sample; means for determining the amount of IL-10 or IL-10R in the sample; and means for comparing the amount of IL-10 or IL-10R in the sample with a standard. The standard is, in some embodiments, a non-cancer cell or cell extract thereof. The compound or agent can be packaged in a suitable container. The kit can further comprise instructions for using the kit to detect cancer in a sample.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### Examples

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### IL-10R/IL-10 Project Overview

A goal of this project is to generate IL-10-R1 and IL-10 blocking antibodies to overcome resistance to immunotherapy across solid cancers. In embodiments, one indication is solid tumors. This work is clinically differentiated because it involves a new immunotherapy that inhibits the IL-10R/IL-10 signaling pathway, as well as in combination with a therapeutic that stimulates immune cells.

### Target Biology and Therapeutic Concept

Cancers refractory to immune checkpoint (PD-1, PD-L1, and CTLA-4) blockade fail to mount significant anti-tumor T lymphocyte responses. Since antigen presenting cells (APCs) are responsible for priming T lymphocytes, lack of adequate APC activation is one mechanism underlying resistance to checkpoint blockade. Low-dose mouse IL-10R1 blockade combined with a mouse CD40 agonist and MPL consistently eradicated injected tumors and controlled or eradicated large non-injected tumors. Cured animals demonstrated long-lasting immunity by resisting tumor re-implantation at 90 days without further treatment. The therapeutic concept is to generate potent anti-hIL-10R1/hIL-10 blocking antibodies in combination therapy with immunostimulatory agents.

### Discussion and Next Actions

huNSG mouse model is a challenging model that has significant variability. PI is reconstituting the mice with mixed donor cells to hopefully reduce variability. The translation of mouse to human needs to be further understood.

Without wishing to be bound by theory, embodiments comprise generating MK-1966 as a benchmark IL-10 therapeutic antibody, EC50 ranking by signaling blockade assay (pSTAT3) for IL10 candidate against benchmark, initiating developability studies for one IL10 & one IL10R candidate, and initiating in vivo studies.

For the IL-10 project, without wishing to be bound by theory, embodiments comprise graduating the IL-10 candidate with an in vitro data package, including DC activation, T-cell co-culture, pSTAT3, KD determination, and manufacturability assessment. For the IL-10R project, next steps include validating a new candidate, such as by reevaluating a backup candidate (15A02) that came from a wild-type mouse; affinity maturing 43C05 to cross-react with cyno; and/or starting a new discovery campaign.

Aspects of the invention comprise validation of an αIL-10R candidate, such as 43C05 (AlivaMab®). Attenuated binding to cynomolgus receptor was observed. No “red-flags” were identified during the developability study.

Embodiments of the invention also comprise the identification and optimization of an αIL10 candidate. The final humanized αIL10 candidate selected was 49G08_HIL2. The liability site as identified in sequence analysis was engineered out. No “red-flags” were identified during developability study for the humanized candidate. A manufacturability study of the candidate has been initiated.

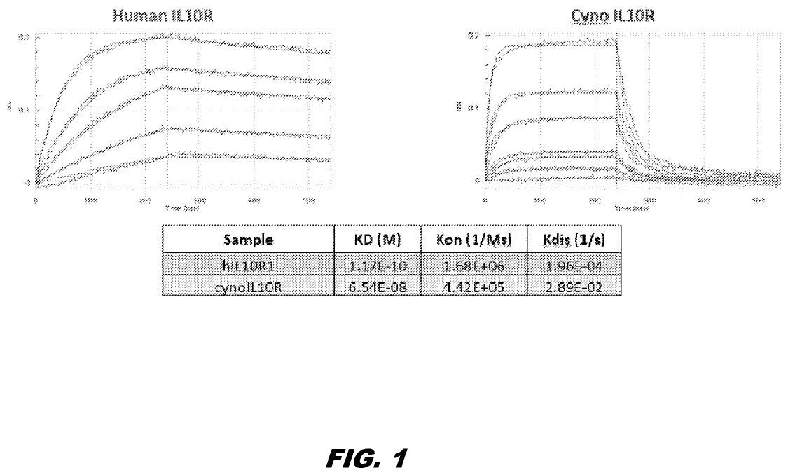

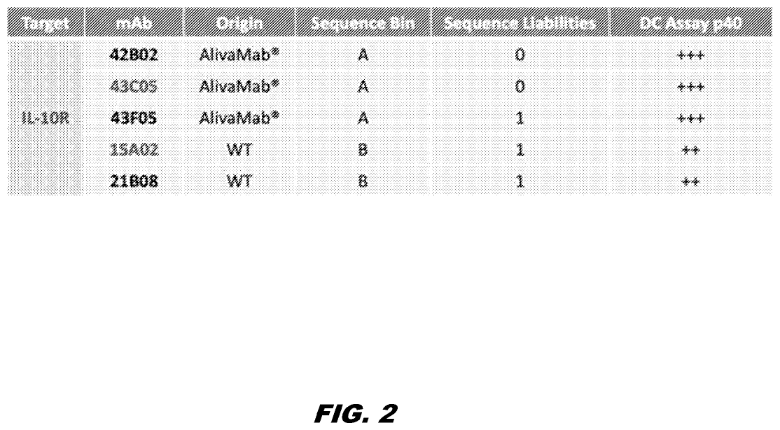

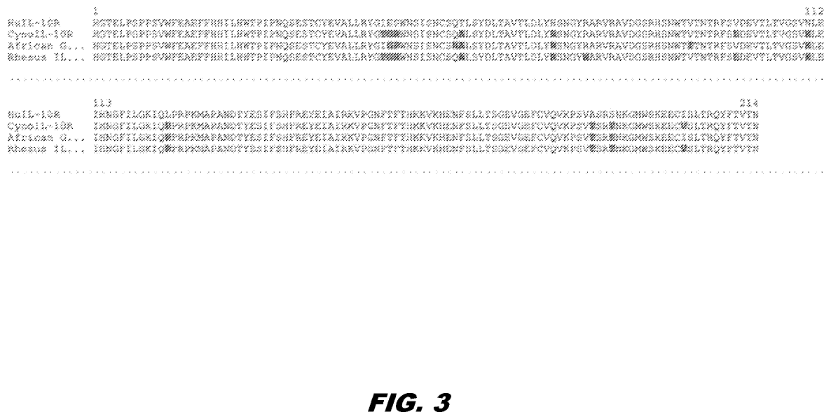

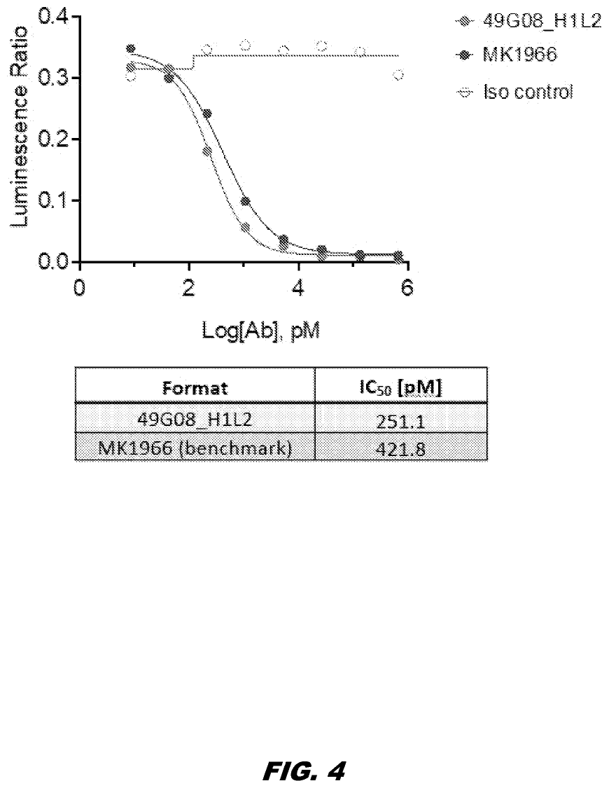

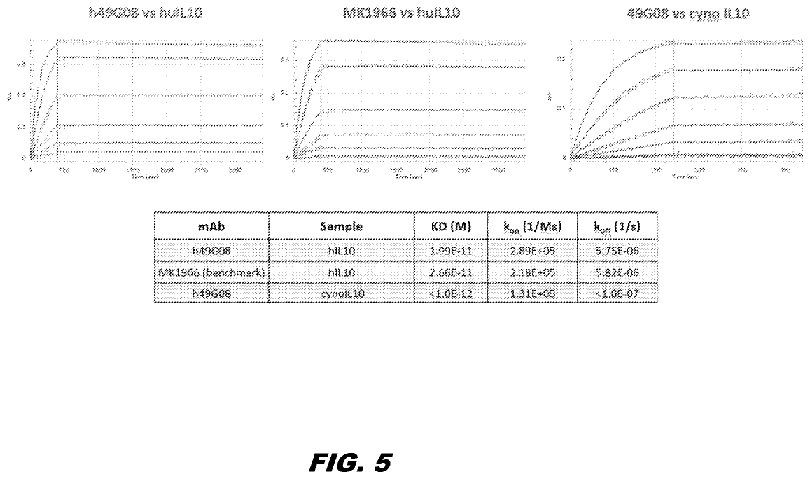

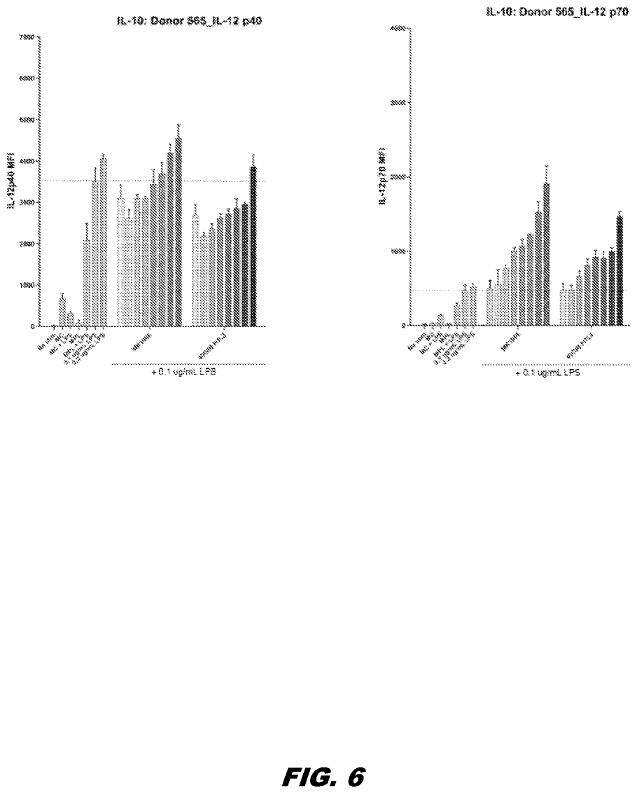

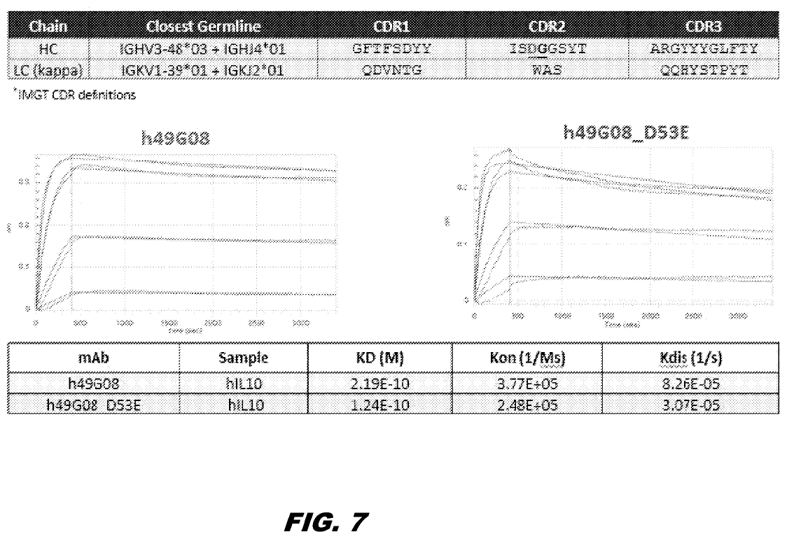

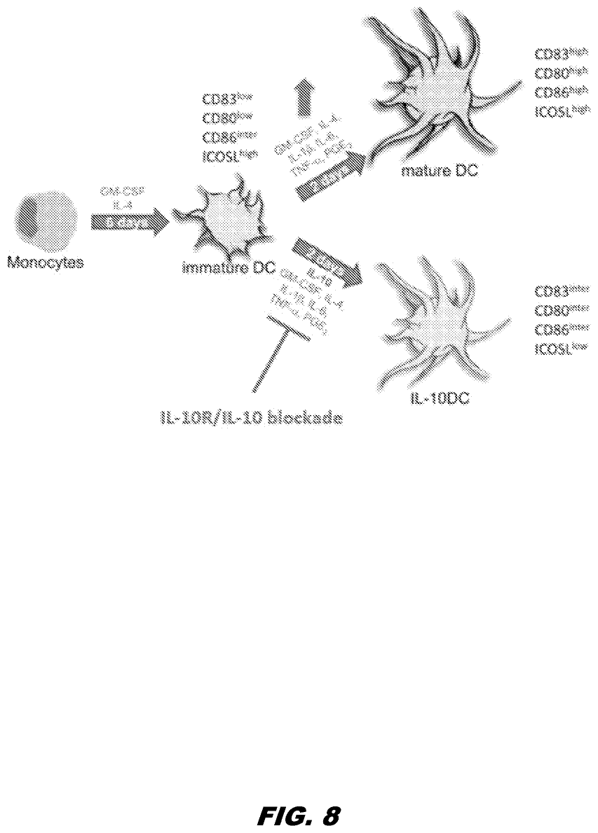

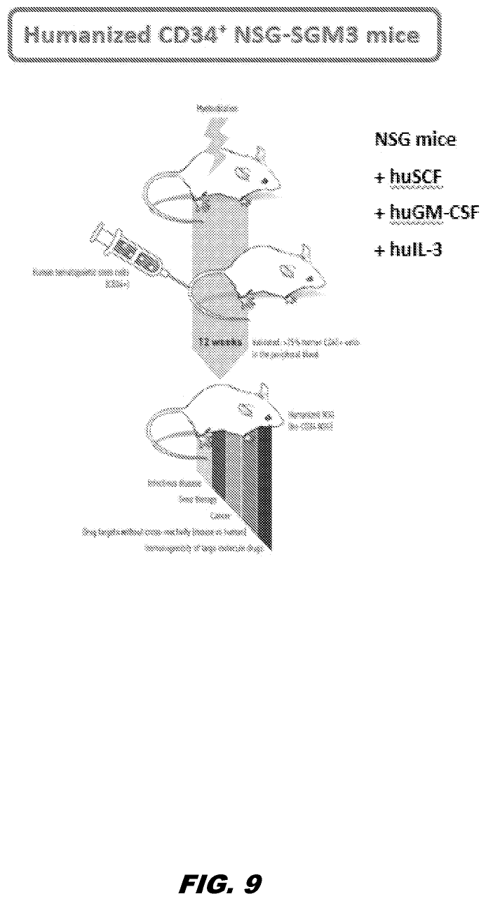

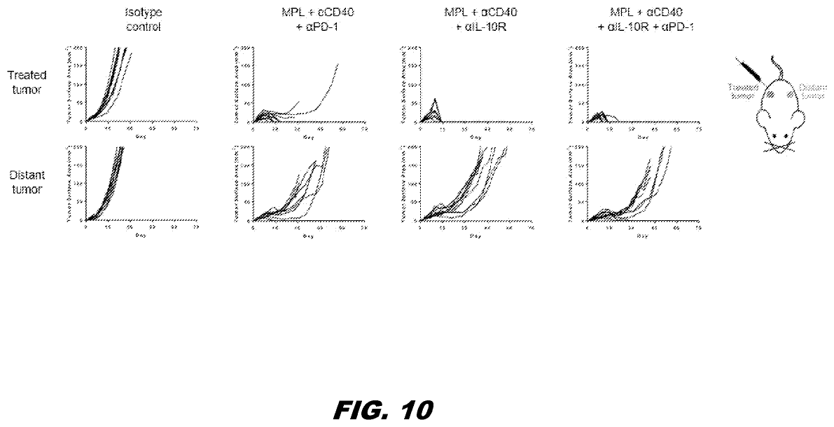

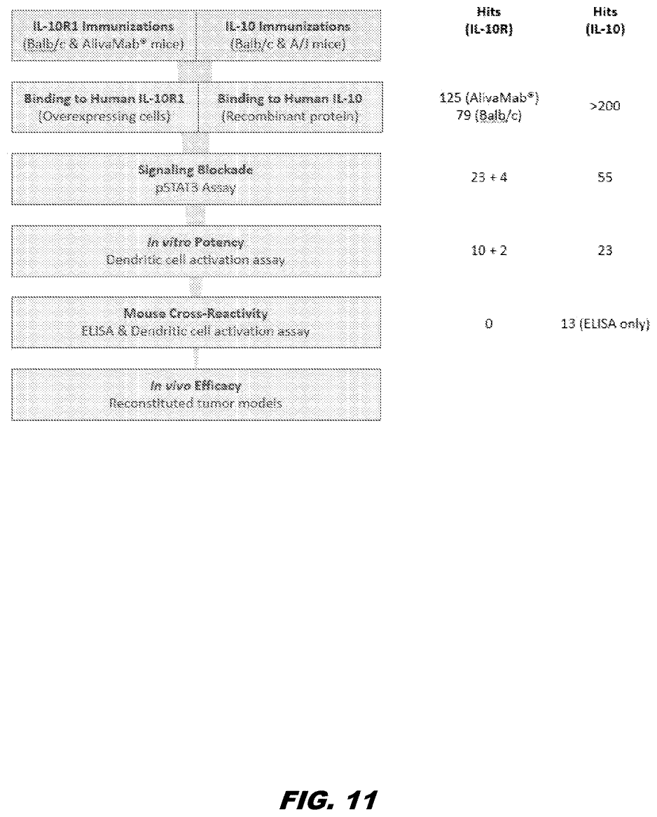

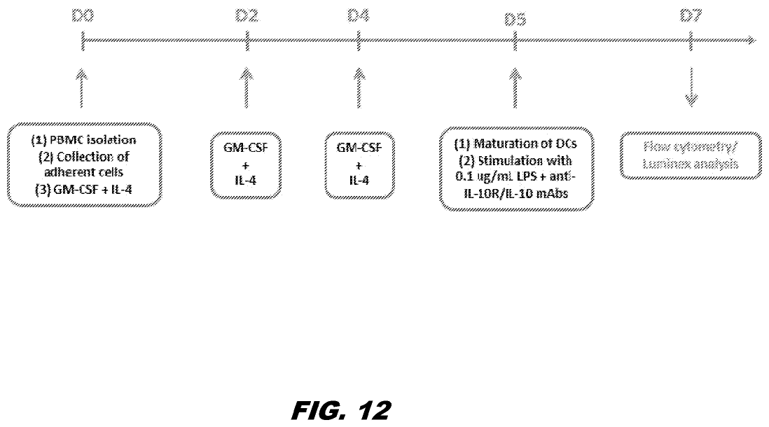

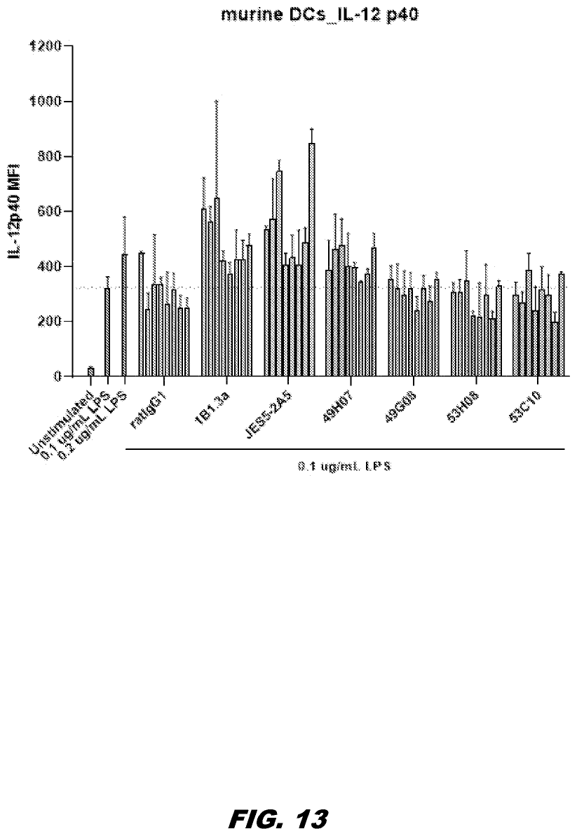

FIG. 1 provides data showing binding of αIL-10R candidate to the cynomolgus IL10R. 100× difference in binding affinity prohibits preclinical toxicology assessment. FIG. 2 represents an evaluation of backup molecules. FIG. 3 provides sequences showing different NHP species for preclinical toxicology assessment. Sequence similarity above 94% and most mutations are conserved amongst. Similar binding deficiency as for cynomolgus IL10R observed with African green monkey IL10R. FIG. 4 provides data showing humanized αIL10 candidate blocks STAT3 signaling pathway more efficiently than the benchmark. FIG. 5 provides data showing the binding affinity of humanized the αIL10 candidate. FIG. 6 illustrates the humanized αIL10 candidate performs similarly to the benchmark in DC activation assay. MAB2171: anti-human IL-10 (R&D); MC: Maturation cocktail (GM-CSF, IL-4, IL-1b, IL-6, TNFa, PGE-2); MPL: monophosphoryl lipid A, 10 μg/mL; LPS: lipopolysaccharide, 0.1 ug/mL. FIG. 7 provides data showing optimization of h49G08. 49G08 CDR-H2 contains an aspartate isomerization site in CDRH2; the motif is not present in the germline. D53E mutant was generated to engineer out liability site. hu49G08_D53E variant passed forced stress developability assessment and is currently being evaluated for manufacturability FIG. 8 provides a schematic showing the generation of tolerogenic and immunogenic dendritic cells from monocytic precursors (adapted from Hubo et al, Frontiers Immunol 4, 82 (2013)). FIG. 9 provides a schematic showing animal studies, including humanized mouse models to validate in vivo efficacy of IL10/IL10R mAb lead candidates (adapted from The Jackson Laboratory). FIG. 10 provides data showing the validation of the therapeutic concept with mouse tool mAbs. The validation included syngeneic B16F10 melanoma model, bilateral tumor injection (treatment on one side), and an injection of Monophosphoryl Lipid A (MPL), CD40 agonist Ab, IL-10R blocking Ab, and PD-1 blocking Ab. FIG. 11 provides a schematic showing the IL-10R & IL-10 antibody discovery strategy. FIG. 12 shows a schematic of the dendritic cell activation assay. The assay included healthy donor PBMCs, differentiation of monocyte-derived dendritic cells (moDCs), and 48 h stimulation with 0.1 μg/mL LPS and IL-10R1/IL-10 mAbs. FIG. 13 shows data for the IL-10 antibodies: Mouse Dendritic Cell Activation Assay. Stimulation with LPS in the presence of anti-IL-10 antibodies (0.01-100 μg/ml). Controls: anti-mouse IL-10R mAb (1B1.3A), anti-mouse IL-10 mAb (JES5-2A5). TDI IL-10 antibodies show minimal or no activity in mouse DC assay. FIG. 14 provides the IL-10R/IL-10 Top Hits and Candidate Selection.

### Example 2

### Significance

An important goal of cancer immunotherapy research is to develop new treatments that produce more specific antitumor responses than existing forms of immunotherapy such as immune checkpoint blockade (ICB). One benefit of this can be to decrease the likelihood of toxicities that result from an immune response against healthy tissues. Therapeutic anti-cancer vaccination is intended to produce a highly specific anti-tumor immune response.⁷ However, this approach often requires the identification and synthesis of neoantigens associated with malignant cells, a timeconsuming and costly process.⁸ We⁶ and others^{9,10} have sought to circumvent this challenge by using the tumor itself to direct a specific antitumor response through a process known as in situ vaccination (ISV). ISV involves direct injection into growing tumors of agents that activate antigen presenting cells (APCs). Intratumoral APCs, which are exposed to tumor-associated antigens at baseline, then prime T cells to the same antigens to target and kill cancer cells elsewhere.

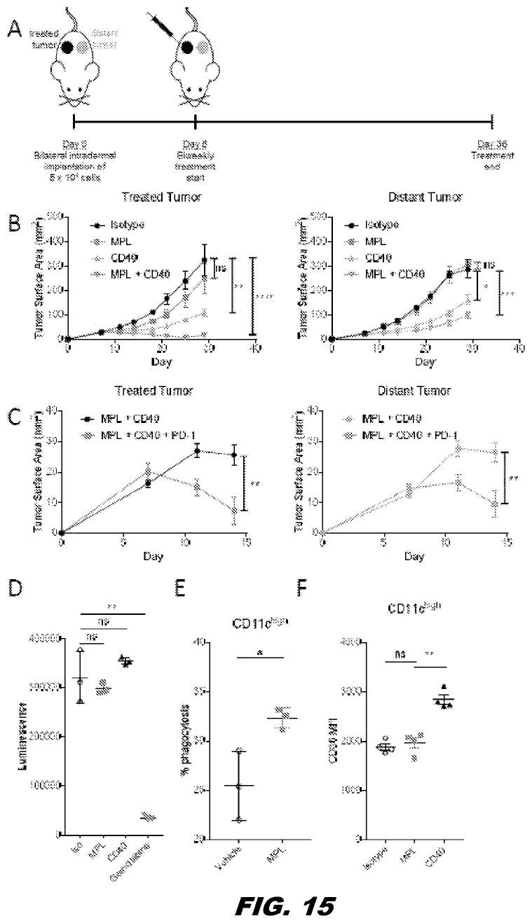

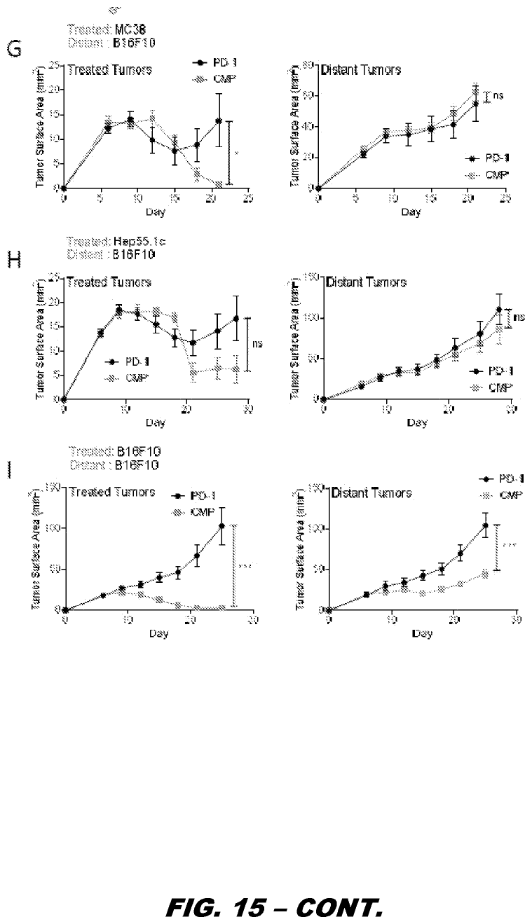

We screened various approaches to ISV in a bilateral tumor model in which mice bearing established B16F10 syngeneic melanoma tumors were injected in one of two tumors (FIG. 15, panel A). The combination of a CD40 agonist monoclonal antibody (mAb) and the TLR4 agonist monophosphoryl lipid A (MPL) controlled treated and distant tumors (FIG. 15, panel B). In this system, shown to be resistant to αPD-1 treatment,^{11,12} αCD40 with MPL (CM) also restored sensitivity to systemic (intraperitoneally administered) αPD-1 (FIG. 15, panel C). We confirmed that αCD40 and MPL are not directly cytotoxic to B16F10 cells in vitro (FIG. 15, panel D). We also observed that MPL augments phagocytosis by CD11c^{high} cells (a population of APCs enriched for dendritic cells [DCs]; FIG. 15, panel E), and that αCD40 increases APC activation (FIG. 15, panel F). These findings support a model in which MPL and αCD40 in combination (i) increase the phagocytosis of tumor antigens by APCs and (ii) induce the activation of these APCs. Encouragingly, we found that when the treated tumor was replaced with a different syngeneic tumor (from the colorectal cancer cell line MC38, or the hepatoma cell line Hep55.1c) efficacy at the distant B16F10 tumor was lost (FIGS. 15, panels G, H) whereas it was maintained when both tumors were B16F10 (FIG. 15, panel I). This indicates that (i) the identity of the treated tumor impacts the specificity of the distant response, and (ii) direct contact of αCD40 and/or MPL with the distant B16F10 tumor is not mediating its control.

### Rigor and Transparency of Prior Work:

Diverse forms of ISV have been studied, including in seminal preclinical studies from others upon which this work largely is based.^{9,10} Strengths of that work include the use of randomization and mycoplasma testing (an important contaminant, especially in immunologic studies¹³). The use of blinding¹⁰ and cell line validation^{9,10} has also been transparently reported. As discussed below, we will take these steps and others to maximize scientific rigor, and report these steps transparently to ensure that our results are reproducible.

### Plan to Overcome Known Barriers:

ISV triggers negative feedback mechanisms involving the secretion of inhibitory cytokines (e.g., TGFβ, IL10) that suppress APC activation^{14,15} and APCs' ability to trigger an immune response.^{16,17} Yet ISV that includes blockade of these pathways, as a component of the in situ vaccine itself, has not been previously studied to our knowledge. The local secretion of IL10 in response to ISV represents a critical barrier to optimal efficacy. Here we will validate in situ vaccines that include blockade of the IL10-IL10 receptor (αIL10R) axis to overcome this barrier.

Findings with αIL10R:

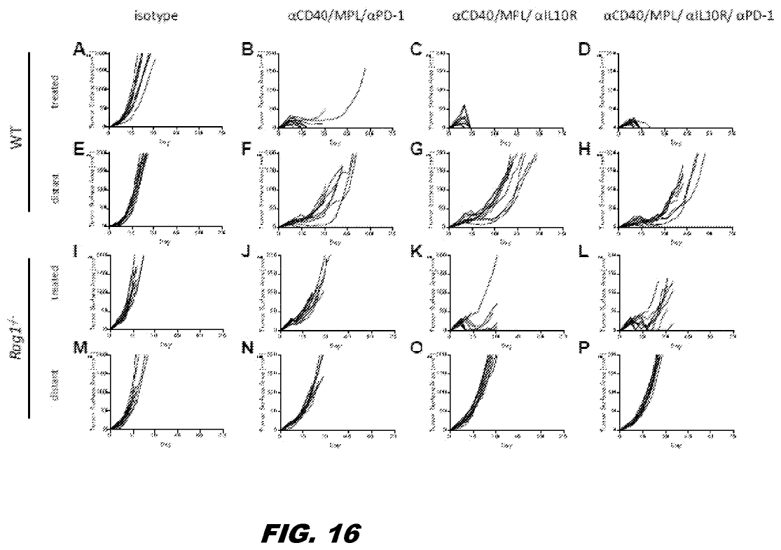

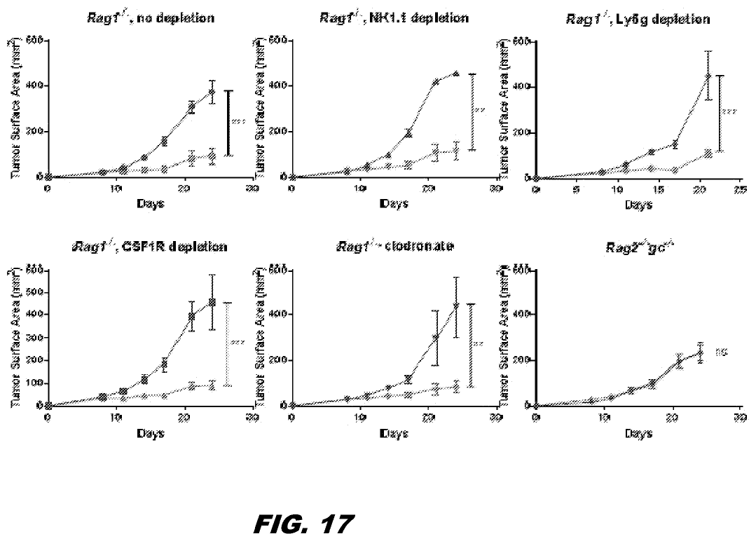

IL10 is a pleiotropic cytokine.^{18,19} Both blockade and stimulation of IL10R have been explored as cancer therapies.^{20,21} We found that intratumoral treatment with αIL10R alone had no effect on tumor growth. Nevertheless, intratumoral treatment with αIL10R plus αCD40/MPL (CM), led to full eradication of treated tumors (FIGS. 16, panels C, D) and improved control of distant tumors (FIGS. 16, panels G, H), even obviating the need for PD-1 blockade (FIGS. 16, panels C, G). Tumor control was lost in distant tumors in Rag1^{-/-} mice lacking functional T and B cells (FIGS. 16, panels M-P). Strikingly, Rag1^{-/-} mice treated with αIL10R-containing regimens had complete but transient elimination of many treated tumors (FIGS. 16, panels K, L). These data indicate that blockade of IL10R not only augments ISV with CM but induces rapid control of treated tumors independently of adaptive immune cells such as T cells.

To understand which cells mediate the effect of αCD40, MPL, and αIL10R (CMI) at the treated tumor, we implanted unilateral tumors into Rag1^{-/-} animals depleted for natural killer (NK) cells (with αNK1.1²²), neutrophils (with αLy6g²³), as well as macrophages, monocytes, and other phagocytic cells (with αCSF1R²⁴ and clodronate25 respectively). Therapeutic efficacy was maintained in these animals but lost in Rag2^{-/-}gc^{-/-} mice, which lack functional receptors for a subset of cytokines and specific immune cell types (FIG. 17). The only cell types deficient in Rag2^{-/-}gc^{-/-} mice but not deficient in Rag1^{-/-} mice are NK cells and other innate lymphoid cells (a group of lymphocytes that do not express T-cell receptors or antibodies, ILCs).²⁶ Furthermore, depletion of NK cells did not eliminate CMI efficacy. These data indicate that regression of tumors treated with CMI can be mediated by non-NK ILCs.

### Innovation

This example has the following unique and innovative aspects pertaining to how the innate immune system can be recruited to augment the response to cancer immunotherapy:

#1. Data show that non-NK ILCs directly control tumors in vivo.²⁷ Without wishing to be bound by theory, we will validate whether blockade of IL10R augments non-NK ILC killing of tumor cells, as indicated by our data (FIG. 17). If so, this will be the first therapy to our knowledge (ISV or otherwise) shown to enhance the anti-tumor activity of these cells.

#2. We will characterize αIL10R within ISV as a new approach to enhancing ISV that (while conferring systemic immunity) induces rapid, complete regression of injected tumors (FIGS. 16, panels C, D). Without wishing to be bound by theory, this can be used for locally advanced cancers that are not curable with surgery or radiation.

#3. The secretion of immunosuppressive cytokines during ISV represents a critical barrier to the systemic and local efficacy of this therapy. To our knowledge, our studies will be the first to validate blockade of an inhibitory APC receptor as an integral component of ISV.

### Approach

### Part 1. Validate how IL10 Modulates the Direct Anti-Tumor Activity of Innate Immune Cell Subsets.

### Rationale:

While a critical goal of ISV is to stimulate a systemic anti-tumor response, rapid and robust control of the treated tumor is also of crucial therapeutic importance in a discreet subset of most solid cancers. In many unresectable locally advanced cancers (for instance, 30% of pancreatic adenocarcinomas at initial diagnosis28), such an approach can represent the only curative option. This aspect of ISV, which has received relatively little attention, thus merits further research.

### Strategy:

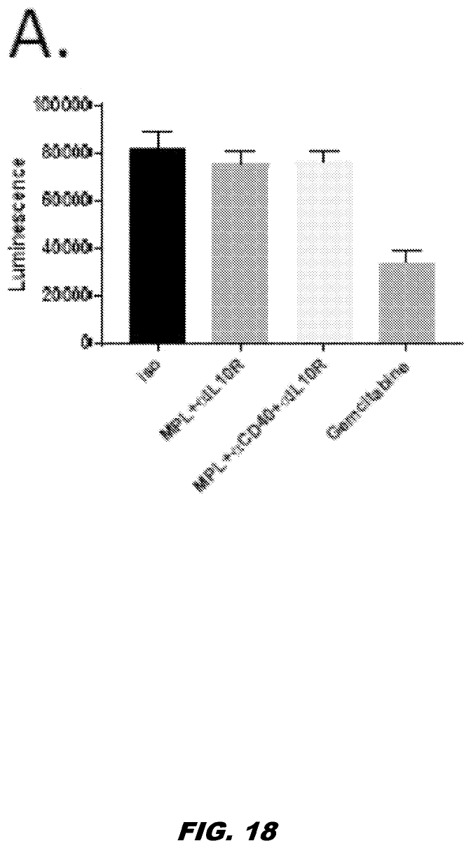

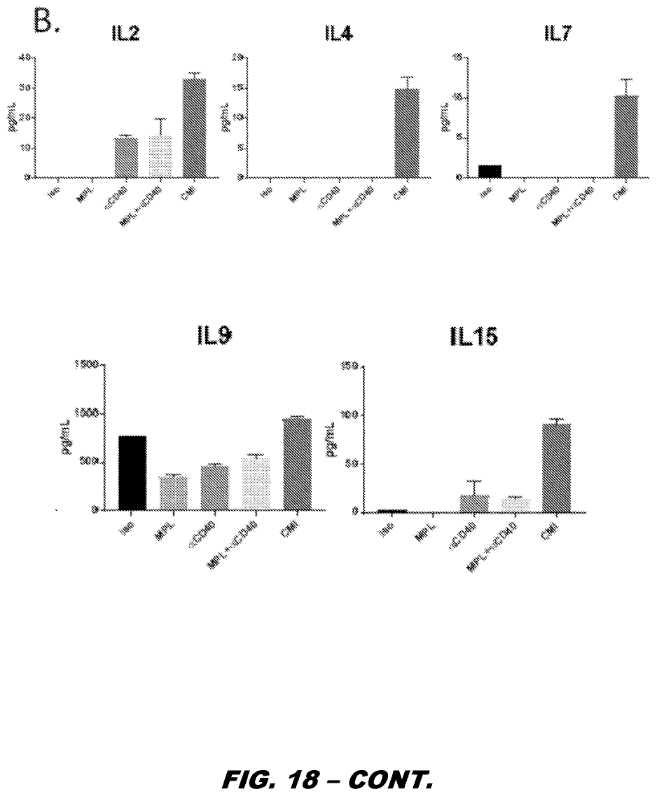

We have found that αIL10R-containing ISV treatments, while not appreciably cytotoxic in vitro (FIG. 18, panel A), confer rapid and durable elimination of the treated tumor (FIGS. 16, panels C, D). Furthermore, while rapid regression is not dependent on adaptive immunity (FIGS. 16, panels K, L), the durability of this effect is (FIGS. 16, panels C, D). The results shown in FIG. 17 indicate the involvement of non-NK ILCs. In this experiment, NK cells, neutrophils, monocytes, and macrophages were depleted 1 day prior to treatment.²⁹

To ensure that our assessment of immune cell changes induced by blockade of IL10R within treated tumors is unbiased, we will harvest isotype control, αCD40/MPL (CM), and αCD40/MPL/αIL10R (CMI) tumor at 24 hours, when differences in treatment and control of tumor growth is first detectable, for assessment by single-cell RNA sequencing (scRNA-Seq). Collection and analyses will be performed as previously described^{30,31}. These samples will be assessed for relative abundance of immune cells, subsets of which can be more precisely identified with this approach,³¹ and for functional changes in gene expression (e.g., expression of granzyme B in ILC1s, which have previously been shown to control tumor growth²⁷). These findings will subsequently be confirmed using flow cytometry to assess changes in the populations of interest, and to assess their changes over time (6, 12, 24, and 48 hours after treatment).

Based on these experiments, we will next focus on one or more innate immune cell populations of interest and test their direct anti-tumor activity in vivo and in vitro. For phagocytic cells (e.g., neutrophils, macrophages), we will conduct in vitro and in vivo phagocytosis assays using FITC-labeled beads,⁶ as shown in FIG. 15, panel E, and using GFP-expressing B16 cells readily available in our laboratory. Using previously published assays,³² we will implant GFP-expressing B16 cells into syngeneic C57BL/6 mice treated intratumorally with isotype control, CM, or CMI. We will then harvest tumors at 6, 12, 24, and 48 hours after treatment and use flow cytometry to determine which phagocytes consume malignant cells (as previously described³²) differentially upon addition of αIL10R. For in vitro confirmation, and to exclude indirect effects from other immune cell populations, we will harvest and sort phagocytic cells from wildtype C57BL/6 mice (from bone marrow [as in FIG. 15, panel E], from blood, or from tumor, depending on the population of interest), coculture these cells with GFP-expressing B16 cells, and treat as described herein. We will determine by flow cytometry whether phagocytosis of tumor cells is differentially impacted over time by the addition of αIL10R.

To assess the direct killing of tumor cells by innate immune cells differentially affected by αIL10R in the experiments described herein, we will use an in vitro clonogenic killing assay.⁶ Immune cells of interest will be harvested and cocultured with tumor cells as described herein. In vitro samples will be treated as described for the phagocytosis assays above and killing will be quantified at 24 and 48 hours. Of note, this approach was first established for phagocytic immune cells³³ and later adapted for nonphagocytic immune cells.³⁴ Given our results (FIG. 17), without wishing to be bound by theory, our regimen enhances tumor-cell killing by ILCs.

### Part 2. Ascertain Whether Modulation of Innate Immune Cells by IL10 Impacts the Priming and Function of Anti-Tumor T Cells.

### Rationale:

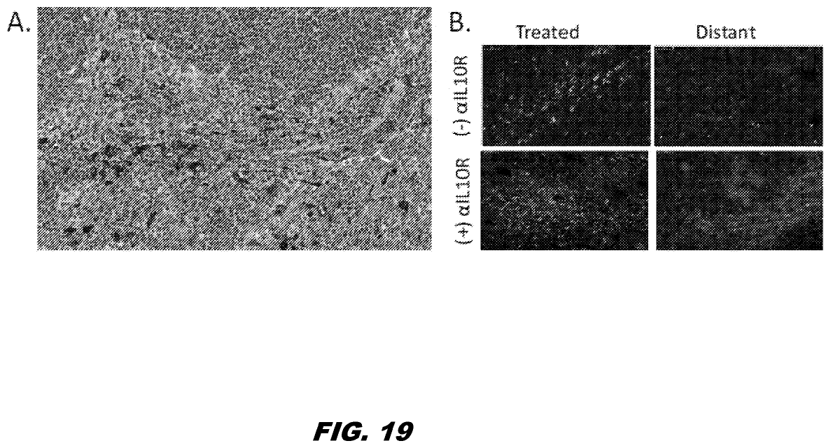

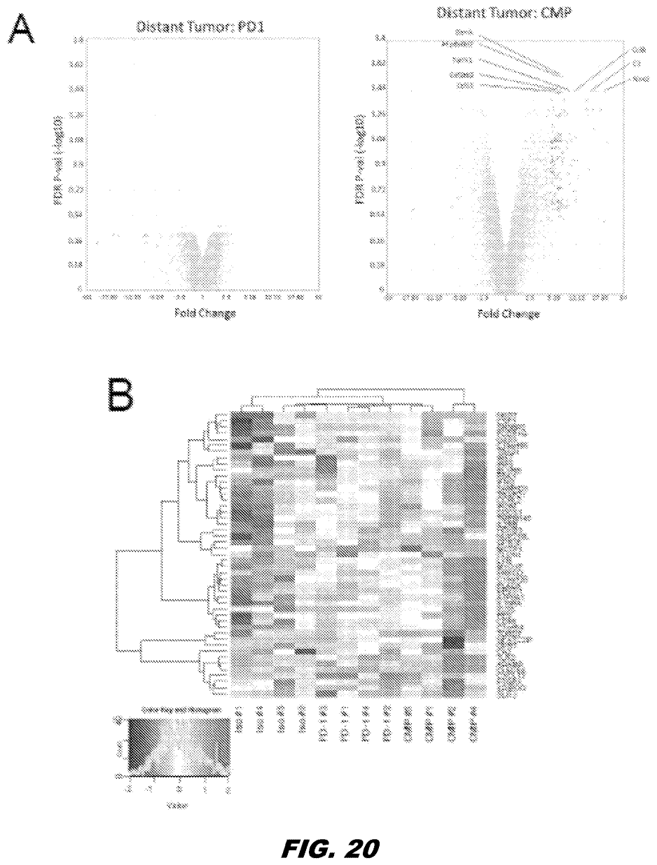

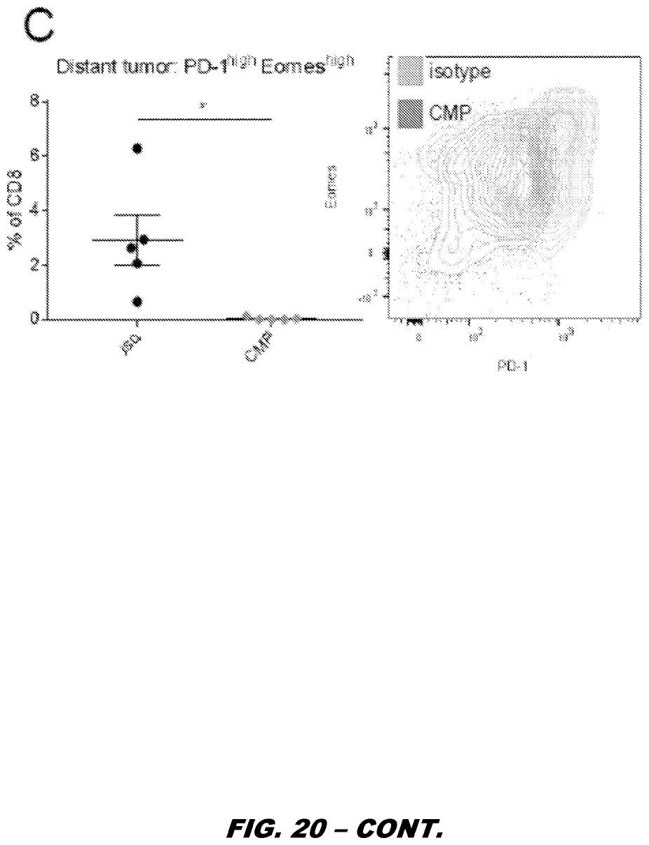

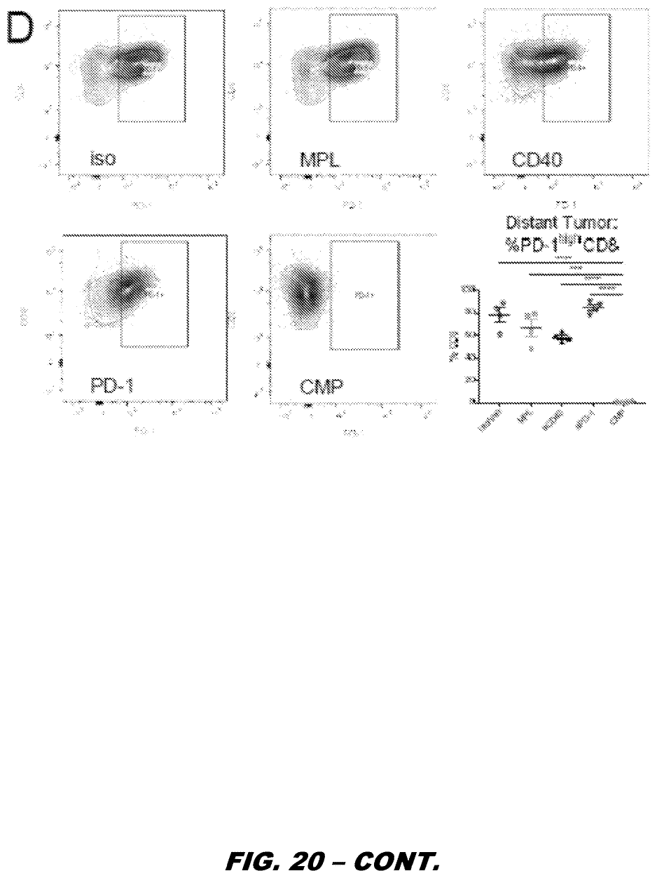

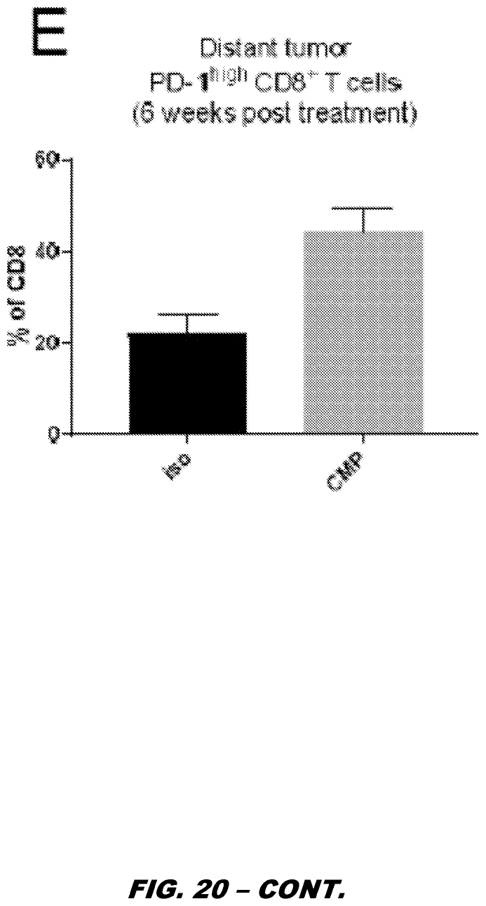

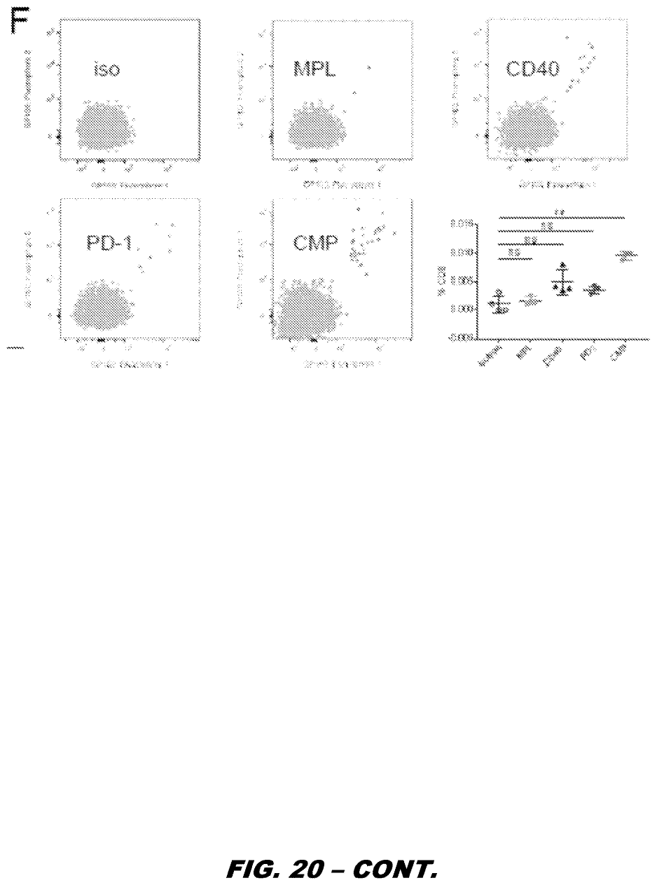

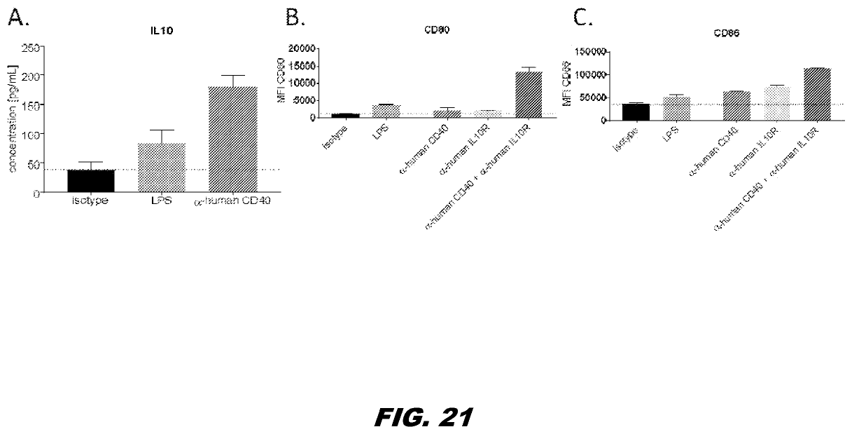

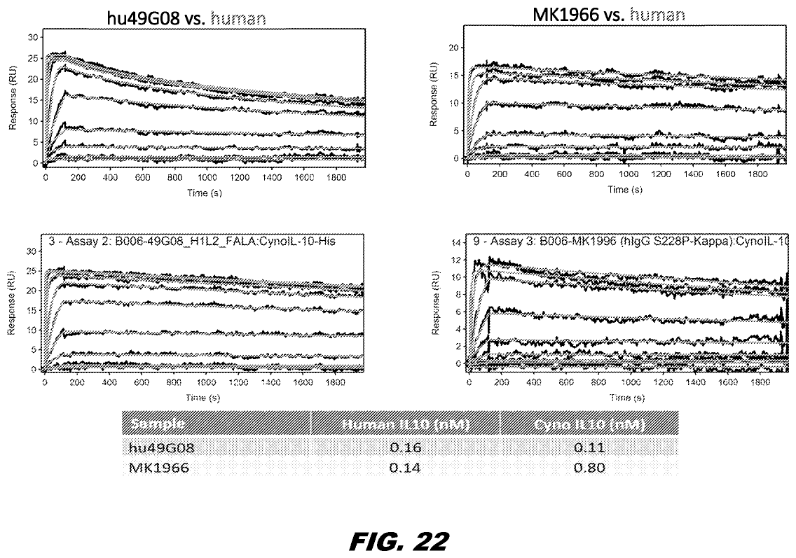

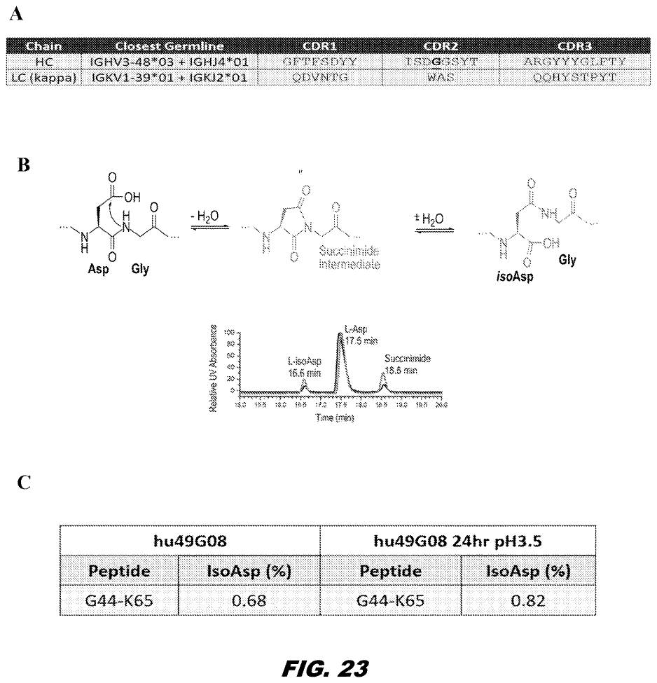

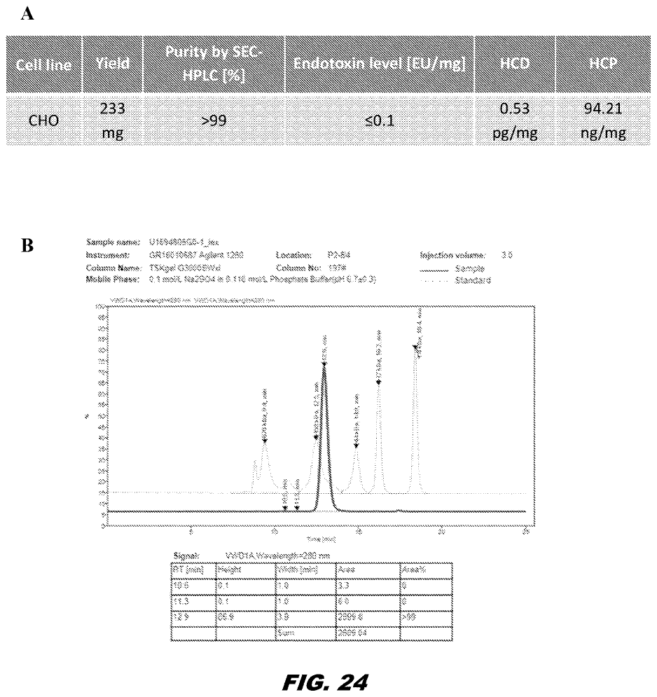

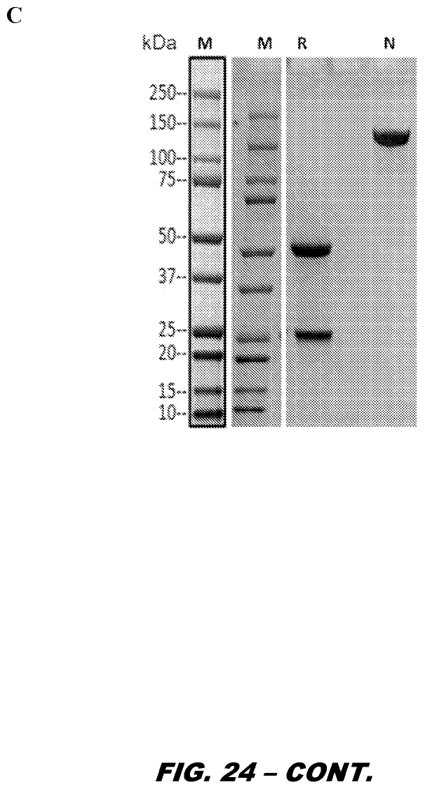

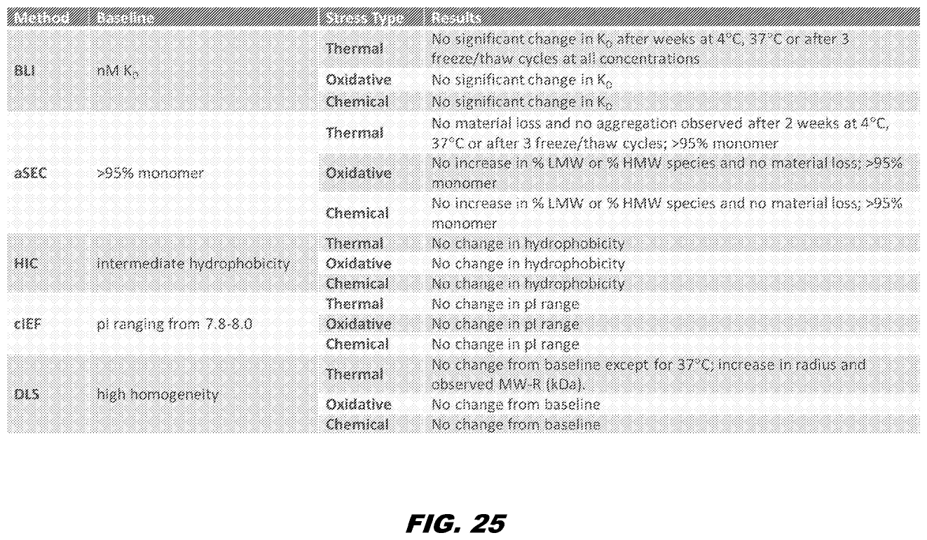

As a negative feedback mechanism for APC stimulation, IL10 inhibits APCs' ability to prime T cells.¹⁸ This mechanism can limit optimal ISV efficacy in the clinic. Using H&E staining of spleens from B16F10-bearing mice, we observed that CMI (but not CM) induces the accumulation of melanin-containing cells with a dendritic morphology (the specific cell type cannot be determined by H&E) in the periarteriolar lymphoid sheath (PALS), a T-cell rich region to which activated dendritic cells migrate and present antigen³⁷ (FIG. 19, panel A). Furthermore, immunofluorescence assays showed that the addition of αIL10R to ISV augments the accumulation of CD8^{l} cytotoxic T cells in treated and distant tumors (FIG. 19, panel B).

### Strategy:

By testing mice bearing bilateral B16F10 tumors (FIG. 15, panel A), we have found that intratumoral CD40 and MPL (CM) injection when combined with systemic PD-1 blockade (CMP) substantially changes the immune composition of treated and distant tumors. RNA profiling of distant tumors 1 week after starting CMP treatment showed transcriptional changes consistent with robust T-cell activity. For example, granzyme K, a gene expressed in activated T cells and associated with cytolytic activity,^{38,39} was the most significantly upregulated gene in distant tumors after CMP treatment, whereas it was not significantly upregulated with αPD-1 treatment alone (FIG. 20, panel A). We will perform a modified version of this experiment, comparing CM to CMI, to establish the role of IL10R blockade. Instead of assessing bulk tumors, we will conduct scRNA-Seq, as described herein, to analyze distant tumors at 1 week. Phenotypic changes specifically in T cells will be interrogated in Part 2, as will such changes in CD45+ immune cells infiltrating the distant tumor.

Next, we will determine the role of αIL10R in T-cell exhaustion, a key barrier to effective immunotherapy.⁴⁰ Using annotated gene sets from the Molecular Signatures Database⁴¹ defined as genes upregulated in effector versus exhausted CD8″ T cells,⁴¹ we previously found that samples from animals treated with αPD-1 monotherapy (which is known to reinvigorate a subset of exhausted T cells⁴²) appeared less exhausted than those treated with isotype control, and segregated away from those samples by unsupervised clustering (FIG. 20, panel B). Strikingly, four samples treated with CMP for 1 week were shifted further away from an exhausted expression pattern compared to those treated with αPD-1, despite intratumoral administration of therapy into the contralateral tumor (FIG. 20, panel B). These results were supported by flow cytometry analyses (FIG. 20, panel C), which showed that the terminally exhausted43 PD-1^{high}Eomes^{high} population was markedly reduced upon CMP treatment. We confirmed that this effect was not merely caused by cross-blocking of the staining antibody by showing that αPD-1 monotherapy (or any constituent monotherapy) did not cause loss of the PD-1^{high} population (FIG. 20, panel D). Despite the marked reduction in this exhausted population 1 week after CMP treatment, this effect was lost by 6 weeks (FIG. 20, panel E).

During the K08 we will conduct this type of experiment to compare CM with CMI and determine how αIL10R impacts (i) the persistence of this effect at 4, 6, and 8 weeks, (ii) other markers of T-cell exhaustion (e.g., TIM3, LAG3, and 2B4), (iii) markers of T-cell function and proliferation (e.g., by assaying the expression of granzymes and Ki67 by flow cytometry), and (iv) ex vivo killing, which will be used for functional confirmation as previously described,³⁴ and discussed in Part 1. We will go on to assess the specificity of the T-cell response to CMI therapy, using the EliSPOT assay as we previously described,⁶ and the adoptive transfer of Pmel-1 TCR transgenic T cells which are specific to the melanoma differentiation antigen gp100.44 To determine how CMI affects the relative abundance of endogenous T cells that recognize gp100, we will use combinatorial encoding of MHC multimers.⁴⁵ Using this technology, we previously found⁶ that CMP significantly expands this rare population of anti-tumor T cells (FIG. 20, panel F). We will go on to determine the effect of adding αIL10R and ask whether systemic PD-1 blockade is necessary for this expansion in the setting of IL10R blockade. To validate the effect of IL10R blockade on immunologic memory, CM-treated and CMI-treated animals initially bearing unilateral B16F10 tumors will be reimplanted with B16F10 tumors at 90 days post-therapy and assessed for outgrowth of new tumors. To validate whether control of distant tumors by CMI depends on T-cell priming, we will block the interaction between CD28 and CD80/86 (so-called signal 2, a component of T-cell priming) with blocking mAbs as previously described in vivo.⁴⁶⁻⁴⁸

Once the experiments are completed, we will focus on the results that are impacted by the presence of αIL10R in a statistically significant manner. We will then test whether the result is still observed in the absence of the following populations (all of which have been shown to possess APC function⁴⁹⁻⁵³): cross-presenting Batf3+ DCs (using Batf3^{-/-} mice⁴⁹), B-cells (using CD20 depletion⁵⁴), macrophages (using CSFIR depletion²⁴), neutrophils (using Ly6g depletion²³), and ILCs (using CD90 depletion²⁹). This will indicate whether the impact on T cells is dependent on any of these immune cell populations.

Without wishing to be bound by theory, the addition of αL110R to CM will increase the tumor-specific T-cell response and lead to regression of contralateral tumors in a T-cell dependent manner. Further, this effect will require priming by cells that have APC function—the same cell type mediating direct tumor killing as discussed in Part 1. Given the ability of pegylated IL10 (pegilodecakin) to enhance anti-tumor T-cell activity,²¹ we will test whether αIL10R can directly inhibit anti-tumor T-cell activity, using the in vitro killing assay described herein, in the presence and absence of recombinant IL10. We will perform CD8 depletion in CMI-treated mice with bilateral B16F10 tumors to confirm that the effect is dependent on CD8+ T cells. If these results indicate that CMI mediates control of distant tumors by simple diffusion and direct contact (which appears unlikely given the results in FIGS. 15, panels G-I), we will test the efficacy of this regimen as a systemic therapy by intravenous administration, starting in mice bearing B16F10 tumors. Without wishing to be bound by theory, the impact of CMI on the distant tumor can be mediated by B cells or CD4+ T cells. We will deplete these cell populations (using αCD4 and αCD20,⁵⁴ respectively) in CMI-treated animals and assess the impact on distant matched tumors.

### Part 3. Determine the Generalizability of αIL10R as a Means of Augmenting In Situ Vaccination Using Different In Situ Vaccines, Different Tumor Models, and Human Cells.

### Rationale:

The purpose of this Part is to determine whether the enhanced tumor control observed when αIL10R is added to CM (FIG. 16) also occurs when αIL10R is added to other in situ vaccines, and to test whether this approach is effective in other tumor models and human cells.

### Strategy:

We will determine whether αIL10R can also enhance the efficacy of three different in situ vaccines, and whether CMI is effective in three additional tumor models and in human cells (using α-human mAbs we have developed).

We will start with the bilateral B16F10 model (FIG. 15, panel A), combining intratumoral QIL10R with three intratumorally injected in situ vaccines: (i) inactivate vaccinia virus⁵⁵; (ii) an agonistic αOX40 mAb with which our lab has many years of experience⁵⁶ and which has been shown to contribute to systemic tumor control when used for ISV;⁹ and (iii) the TLR9 agonist CpG, chosen because early clinical data in melanoma show that intratumoral stimulation of TLR9 can restore sensitivity to αPD-1 (NCT02680184).⁵⁷ Studying these very different approaches to ISV will elucidate whether the role of αIL10R in ISV can be extended beyond its activity in CMI.

To validate whether αIL10R augments the regimen of αCD40 and MPL (CM) in other cancer models, we will administer CM with and without IL10R in the following models (all in syngeneic C57BL/6 mice): (i) mice bearing bilateral MC38 colorectal tumors, (ii) mice bearing bilateral hep55.1c hepatomas, and (iii) mice bearing a unilateral B16F10 melanoma tumor that will be assessed for lung metastasis. Responses in (i) and (ii) will be quantified with twice weekly tumor measurements, as in FIG. 15, panel A. Responses in (iii), the metastatic seeding model, will be determined by sacrificing animals at 14 and 21 days and quantifying lung metastases, as we have previously described.⁵⁸

Lastly, we will validate α-human CD40 and α-human IL10R mAbs on human cells from healthy donors. These experiments will be performed in samples from male and female donors (and gender will be reported upon publication). We have generated and validated mAbs directed against human CD40 and IL10R. We generated monocyte-derived dendritic cells from the peripheral blood of healthy donors using a previously described protocol in which cells were incubated with GM-CSF and IL4.59 We showed through multi-analyte profiling that stimulation of these cells using α-human CD40 caused secretion of IL10 (FIG. 21, panel A). We next used flow cytometry to assess expression of CD80 and CD86, both markers of APC activation, on these cells. While α-human CD40 modestly upregulated these markers, blockade of IL10R with α-human IL10R and α-human CD40 caused a more pronounced upregulation of these markers (FIGS. 21, panels B, C). To determine whether similar changes occur in innate immune cell populations identified in Part 1, we will perform these experiments on the populations identified there. If Part 2 reveals that these cell types are not directly involved in significant antigen presentation, we will coculture the cells with the dendritic cells shown in FIG. 21 to assess their effect on these APCs.

Without wishing to be bound by theory, IL10R blockade will be an effective way to augment the efficacy of different in situ vaccine regimens in different tumor models and in human cells. If αIL10R fails to enhance the efficacy of the three forms of ISV tested in this Part, treated tumors from those animals will be harvested and subjected to multianalyte profiling. Inhibitory cytokines that are significantly upregulated will then be blocked using validated murine mAbs. Without wishing to be bound by theory, other immunosuppressive cytokines (e.g., TGFβ⁶⁰) confer negative feedback with those treatments, and such results can support the investigation of different agents to enhance their efficacy (e.g., a TGFβ-blocking mAb). If efficacy is limited to cutaneus (intradermal) melanoma, we will harvest the colorectal (MC38) and hepatocellular (hep55.1c) tumors and analyze the CD45+ immune cells within these tumors for expression of IL10R and other suppressive receptors. Without wishing to be bound by theory, different tumor types can be infiltrated by different APC populations with relative differences in their inhibitory receptors indicating which receptors can be blocked in these tumor types to enhance ISV efficacy.

### Statistics, Rigor, and Transparency:

Each experiment will be performed in three replicates. Cell line validation and contaminant testing, as well as genotyping of mouse strains, will be done as described in this example. A permutation test of significance will be performed to determine if an experimental group is superior to a control group. Regarding in vivo experiments, mice will be randomized to a control group or one of the ISV experimental therapies (n=15 per group, stratified by gender using random permuted block size). Tumor assessments will be performed by a laboratory member who is blinded to treatment assignment. Tumor volume, survival, or metastatic behavior will be recorded at regular intervals (as described herein), and results will be presented graphically as mean tumor volume at each time point for each treatment group. We will compare time-tumor volume curves using the area under the curve (AUC), which is interpreted as the total tumor burden, and then perform group comparisons using exact small-sample methods. This entails a rank-sum test for pair-wise comparisons where the reference distribution is generated by permutation.

Our approach to computing the sample size has been previously studied.⁶¹ With 15 mice in the experimental and control groups, the odds need to be 3.5:1 in favor of an experimental group having a lower AUC than the control (5% level two-sided test, 80% power). In some experiments, not all the mice are followed for the full length of the study. The primary reason for censoring is the need to sacrifice an animal as per institutional guidelines. Ignoring this type of informative censoring results in a bias of the AUC based rank test statistic. To account for this bias (informative dropouts), the area under the curve in the two treatment groups (AUC1 and AUC2) are calculated up to the minimum follow-up time for each pair-wise difference. A permutation test is again employed to determine whether the treatment groups are different. If mice are sacrificed at variable follow-up intervals, and the reason for the sacrifice is unrelated to the potential failure time, then the logrank statistic will be used to determine whether the experimental group prolongs survival relative to a control group (using a permutation test procedure). For the experiments described herein: ns, not significant; * P≤0.05; ** P≤0.01; *** P≤0.001; **** P≤0.0001. Statistical assessments will be reviewed. Our efforts to ensure rigor and reproducibility will be transparently reported, for each experiment, in the Methods section of publications resulting from this work. Furthermore, raw data will be publicly reported, including by posting raw genomic data into the Gene Expression Omnibus.

### Example 3

### Protein Immunization Strategy

To generate monoclonal antibodies against IL10R1 AlivaMab® and wild type (Blab c A/J mice) were immunized with recombinant human IL10R1-huFc. Mice were immunized at 3-week intervals for at least 4 times, using 10 μg subcutaneous injections at different sites. Mice with sera exhibiting IL10R1 binding activity were acute boosted with IL10R1 using 10 μg intravenous injection prior to fusion of splenocytes with SP2/0 cells for hybridoma generation.

To generate monoclonal antibodies against IL10 wild type (Blab c A/J) mice were immunized with recombinant human IL10. Mice were immunized at 3-week intervals for at least 4 times, using 5 μg subcutaneous injections at different sites. Mice with sera exhibiting IL10 binding activity were acute boosted with IL10 using 10 μg intravenous injection prior to fusion of splenocytes with SP2/0 cells for hybridoma generation.

### Example 4

Referring to FIG. 22, data showing the binding affinity of 49G08_HIL2 is provided. K_{D} (equilibrium binding constant) was determined using surface plasmon resonance (SPR). Samples were captured onto a Cl sensor chip to minimize avidity effects at three different surface densities, and replicated three times.

Referring to FIG. 23, data showing that 49G08_HIL2 is not prone to isomerization is provided. 49G08_HIL2 was stressed for 24 hr at pH3.5 and then dialyzed in PBS. Peptide digestion followed by MS analysis revealed that less than 1% of 49G08_HIL2 isomerizes in acidic stress conditions. FIG. 23, panel A provides data showing insilco sequence analysis. FIG. 23, panel B provides data showing 49G08 CDR-H2 contains an aspartate isomerization site in CDRH2, and that the motif is not present in the germline. FIG. 23, panel C provides mass spectrometry data showing that less than 1% of 49G08_HIL2 isomerizes in acidic stress conditions.

Referring to FIG. 24, data showing the productivity assessment of 49G08_HIL2 is provided. A two-step purification process (Protein A and AEX) was applied, and residual host cell protein and DNA levels were tested for. FIG. 24, panel A provides data showing the productivity of 49G08_HIL2. FIG. 24, panel B provides data showing SDS-Page results of U1694805G0-1 after AEX (anion exchange chromatography) purification. FIG. 24, panel C provides data showing SEC-HPLC (size exclusion-high performance liquid chromatography) results.

Referring to FIG. 25, data showing the developability assessment of 49G08_HIL2 is provided.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. An isolated monoclonal antibody or fragment thereof that specifically binds to Interleukin-10 (IL-10) or IL-10 receptor, the monoclonal antibody comprising a heavy chain, light chain or a combination thereof.

2. The monoclonal antibody of claim 1, wherein the antibody is an IL-10 blocking antibody or an IL-10 receptor blocking antibody.

3. The monoclonal antibody of claim 1, wherein IL-10 is human IL-10.

4. The monoclonal antibody of claim 1, wherein the antibody is fully human or humanized.

5. The monoclonal antibody of claim 1, wherein the antibody is monospecific, bispecific, or multispecific.

6. The monoclonal antibody of claim 1, wherein the antibody is an IgG.

7. The monoclonal antibody of claim 1, wherein the antibody is selected from the group consisting of 49G08_HIL2, 49G08, 48C11, 49H07, 48C06, 53D07, 53H08, 42B02, 43C05, 43F05, 15A02, and 21B08.

8. The monoclonal antibody of claim 1, wherein the antibody competes with the binding of 49G08_HIL2, 49G08, 48C11, 49H07, 48C06, 53D07, 53H08, 42B02, 43C05, 43F05, 15A02, or 21B08.

9. The monoclonal antibody of claim 1, wherein the antibody comprises a heavy chain variable region (V_{H}) comprising an amino acid sequence as indicated below, or a sequence at least 90% identical thereto:
a.
EVQLVESGGGLVQPGGSLRLSCAASGFTFSDYYMYWVRQAPGKGLEW
VSTISDGGSYTYYPDSVKGRFTISRDNAKNNLYLQMNSLRAEDTAVY
YCARGYYYGLFTYWGQGTLVTVSS;
b.
EVQLVESGGGLVKPGGSLKLSCAASGFTFSDYYMYWVRQTPEKRLEW
VATISDGGSYTYYPDSVKGRFTISRDNAKNNLYLQMSSLKSEDTAMF
YCARGYYYGLFTYWGQGTLVTVSA;
c.
EVQLQQSGPDLVKPGASVRMSCKASGYTFTNYVMHWVKQRPGQGLEW
IGYISPYNDGTKYNEKFKGKATLTSDKSSSTAYMELNSLTSEDSAVY
YCAGGDYSYAMDYWGQGTSVTVSS;
d.
EVQLQQSGPDLVKPGASVKMSCKASGYTFTHYVMHWVKQKPGQGLEW
IGYISPYNDGIKYNEKFKGKATLTSDKSSSTAYMELSSLTSEDSAVY
YCAGGDYSYAMDYWGQGTSVTVSS;
e.
EVQLVESGGGLVKPGGSLKLSCAASGFTFSDYYIYWVRQTPEKRLEW
VATISDGGSYTYYPDSVKGRFTISRDNAKNNLYLQMSSLKSEDTAMY
YCARGYYYGLFTYWGQGTLVTVSA;
f.
QVQLKQSGPSLVQPSQSLSITCTVSGFSLTTYGVHWVRQSPGKGLEW
LGVIWRGGSTDYNAGFMSRLSITKDNSKSQVFFKMNSLQADDTAIYY
CAKNSYYYGGNYYFDYWGQGTTLTVSS;
g.
QVQLKQSGPSLVQPSQSLSINCTVSGFSLTTYGVHWLRQSPGKGLEW
LGVIWRGGSTDYNAAFMSRLSITKDNSKSQVFFKMNSLQADDTAIYY
CAKPYYVYGAMDYWGQGTSVTVSS;
h.
QVQLQESGPGLVKPSETLSLTCAVSGGSLSSDYWTWIRQPAGKGLEW
IGRIFATGTTNYNPSLKSRVTMSIDTSKNQFSLKLNSVTAADTAVYY
CARDDWYFDLWGRGTLVTVSS;
i.
QVQLQESGPGLVKPSETLSLTCTVSGGSISSDYWSWIRQPAGKRLEW
IGRIFTSGSTNYNPSLKSRVTMSVDTSKNQFSLKLNSLTAADTAVYY
CARDDWYFDLWGRGTLVTVSS;
j.
QVQLQESGPGLVKPSETLSLTCTVSGGSISRDYWSWVRQPAGKGLEW
IGRIFTNGVTNYNPSLKSRVTMSVDTSKTQFSLKLSSVTAADTAVYF
CARDDWYLDLWGRGTLVTVSS;
k.
QVQMKQSGPGLVQPSQSLSITCTVSGFSLTTYGVHWVRQSPGKGLEW
LGVIWSGGSTYYNAAFISRLSISKDNSKSQVIFKMNSLQVDDTAIYY
CARWDGYSFYYGMDYWGQGTSVTVSS;
or
l.
QVQLKQSGPGLVQPSQSLSITCTVSGFSLTSYGVHWVRQSPGKGLEW
LGVIWSGGSTDYNAAFISRLSISKDNSKSQVFFKMNSLQADDTAIYY
CARWDGDSFYYAMDYWGQGTSVTVSS.

10. The monoclonal antibody of claim 1, wherein the antibody comprises a light chain variable region (V_{L}) comprising an amino acid sequence as indicated below, or a sequence of at least 90% identical thereto:
a.
DIQMTQSPSSLSASVGDRVTITCRASQDVNTGLNWYQQKPGQAPKLLIY
WASSRQSGVPSRFSGSGSGTDFTLTISSLQPEDFATYYCQQHYSTPYTF
GQGTKLEIK;
b.
DIVMTQSHKFMSTSVGDRVSITCKASQDVNTGVAWYQQKPGQSPKLLIY
WASTRHTGVPDRFTGSGSGTDYILTISSVQAEDLALYYCQQHYSTPYTF
GGGTKLEIK;
c.
DIQMTQSPASLSVSVGETVTITCRVSENIYSNLAWYHQKQGKSPQLLVY
AATNLADGVPSRFSGSKSGTQYSLKISSLQSEDFGSYYCQHFWGTLTFG
AGTKLELK;
d.
DIQMTQSPASLSVSVGETVTITCRVSENIYSNLAWYQQKQGKSPQLLVY
AATNLADGVSSRFSGSVSGTQYSLKINSLQSEDFGSYYCQHFWGTLTFG
AGTKLELK:
e.
DIVMTQSHKFMSTSVGDRVSITCKASQDVSTGVAWYRQKPGRSPKLLIY
WASTRHTGVPDRFTGSGSGTDYILTISSVQAEDLALYYCQQHYSTPYTF
GGGTKLEIK;
f.
DVVMTQTPFSLPVSLGDQASISCRSSQSLVHSNGNTHLHWYLQKPGQSP
KLLIYKVSNRFSGVPDRFSGSGSGTDFTLKISRVEAEDLGVYFCSQSTH
VPPTFGGGTKLEIK;
g.
DVLMTQTPLSLPVSLGDQASISCRSSESIVHSNGNTYLEWFLQKPGQSP
KLLIYKVSNRFSGVPDRFSGSGSGTDFTLKISRVEAEDLGVYYCFQGSH
VPLTFGAGTKLEIK;
h.
EIVMTQSPATLSVSPGERATLSCRASQSVSNNLAWYQERPGQAPRLLIY
GASTRTTGIPARFSGSGSGTEFILTISSLQPEDFAIYYCQQYNEWPWTF
GQGTKVEIK;
i.
EIVMTQSPATLSVSPGERATLSCRASQSVSNNLAWYQQKPGQAPRFLIY
GASTRATGMPARFSGSGSGTEFTLTISSLQSEDFAVYFCQQYNEWPWTF
GQGTKVEIK;
j.
EIVMTQSPATLSVSPGERATLSCRASQSVSNNLAWYQQTPGQAPRFLIY
GASTGATGIPARFSGSGSGTEFTLTITSLQSEDFAVYFCQQYNDWPWTF
GQGTKVEIK;
k.
QAVVTQESALTTSPGETVTLTCRSSTGAVTTSNYANWVQEKPDHLFTGL
IGSTNNRAPGVPARFSGSLIGDKAALIITGAQTEDEARYFCALWYSNHL
VFGGGTKLTVL;
or
l.
QAVVIQESALTTSPGETVTLTCRSSTGAVTTSNYASWVQEKPDHLFIGL
IGGTNNRAPGVPARFSGSLIGDKAALTITGAQTEDEAIYFCALWYSNHL
VFGGGTKLTVL.

11. The monoclonal antibody of claim 1, wherein the monoclonal antibody comprises:
a. a heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
b. a heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
c. a heavy chain with three CDRs comprising the amino acid sequences GYTFTNYV, ISPYNDGT, and AGGDYSYAMDY respectively, and/or a light chain with three CDRs comprising the amino acid sequences ENIYSN, AAT, and QHFWGTLT respectively;
d. a heavy chain with three CDRs comprising the amino acid sequences GYTFTHYV, ISPYNDGI, and AGGDYSYAMDY respectively, and/or a light chain with three CDRs comprising the amino acid sequences ENIYSN, AAT, and QHFWGTLT respectively;
e. a heavy chain with three CDRs comprising the amino acid sequences GFTFSDYY, ISDGGSYT, and ARGYYYGLFTY respectively, and/or a light chain with three CDRs comprising the amino acid sequences QDVSTG, WAS, and QQHYSTPYT respectively;
f. a heavy chain with three CDRs comprising the amino acid sequences GFSLTTYG, IWRGGST, and AKNSYYYGGNYYFDY respectively, and/or a light chain with three CDRs comprising the amino acid sequences QSLVHSNGNTH, KVS and SQSTHVPPT respectively;
g. a heavy chain with three CDRs comprising the amino acid sequences GFSLTTYG, IWRGGST, and AKPYYVYGAMDY respectively, and/or a light chain with three CDRs comprising the amino acid sequences ESIVHSNGNTY, KVS, and FQGSHVPLT respectively;
h. a heavy chain with three CDRs comprising the amino acid sequences GGSLSSDY, IFATGTT, and ARDDWYFDL respectively, and/or a light chain with three CDRs comprising the amino acid sequences QSVSNN, GAS, and QQYNEWPWT respectively;
i. a heavy chain with three CDRs comprising the amino acid sequences GGSISSDY, IFTSGST, and ARDDWYFDL respectively, and/or a light chain with three CDRs comprising the amino acid sequences QSVSNN, GAS, and QQYNEWPWT respectively;
j. a heavy chain with three CDRs comprising the amino acid sequences GGSISRDY, IFTNGVT, and ARDDWYLDL respectively, and/or a light chain with three CDRs comprising the amino acid sequences QSVSNN, GAS, and QQYNDWPWT respectively;
k. a heavy chain with three CDRs comprising the amino acid sequences GFSLTTYG, IWSGGST, and ARWDGYSFYYGMDY respectively, and/or a light chain with three CDRs comprising the amino acid sequences TGAVTTSNY, STN, and ALWYSNHLV respectively; or
l. a heavy chain with three CDRs comprising the amino acid sequences GFSLTSYG, IWSGGST, and ARWDGDSFYYAMDY respectively, and/or a light chain with three CDRs comprising the amino acid sequences TGAVTTSNY, GTN, and ALWYSNHLV respectively.

12. The monoclonal antibody of claim 1, wherein the monoclonal antibody comprises:
a. a V_{H} according to the amino acid sequence of EVQLVESGGGLVQPGGSLRLSCAASGFTFSDYYMYWVRQAPGKGLEWV STISDGGSYTYYPDSVKGRFTISRDNAKNNLYLQMNSLRAEDTAVYYCAR GYYYGLFTYWGQGTLVTVSS or a sequence at least 90% identical thereto, and a V_{L} according to the amino acid sequence of DIQMTQSPSSLSASVGDRVTITCRASQDVNTGLNWYQQKPGQAPKLLIYW ASSRQSGVPSRFSGSGSGTDFTLTISSLQPEDFATYYCQQHYSTPYTFGQGT KLEIK or a sequence at least 90% identical thereto;
b. a V_{H} according to the amino acid sequence of EVOLVESGGGLVKPGGSLKLSCAASGFTFSDYYMYWVRQTPEKRLEWVA TISDGGSYTYYPDSVKGRFTISRDNAKNNLYLQMSSLKSEDTAMFYCARG YYYGLFTYWGQGTLVTVSA or a sequence at least 90% identical thereto, and a V_{L} according to the amino acid sequence of DIVMTQSHKFMSTSVGDRVSITCKASQDVNTGVAWYQQKPGQSPKLLIY WASTRHTGVPDRFTGSGSGTDYILTISSVQAEDLALYYCQQHYSTPYTFG GGTKLEIK or a sequence at least 90% identical thereto;
c. a V_{H} according to the amino acid sequence of EVQLQQSGPDLVKPGASVRMSCKASGYTFTNYVMHWVKQRPGQGLEWI GYISPYNDGTKYNEKFKGKATLTSDKSSSTAYMELNSLTSEDSAVYYCAG GDYSYAMDYWGQGTSVTVSS or a sequence at least 90% identical thereto, and a V_{L} according to the amino acid sequence of DIQMTQSPASLSVSVGETVTITCRVSENIYSNLAWYHQKQGKSPQLLVYA ATNLADGVPSRFSGSKSGTQYSLKISSLQSEDFGSYYCQHFWGTLTFGAG TKLELK or a sequence at least 90% identical thereto;
d. a V_{H} according to the amino acid sequence of EVQLQQSGPDLVKPGASVKMSCKASGYTFTHYVMHWVKQKPGQGLEWI GYISPYNDGIKYNEKFKGKATLTSDKSSSTAYMELSSLTSEDSAVYYCAG GDYSYAMDYWGQGTSVTVSS or a sequence at least 90% identical thereto, and a V_{L} according to the amino acid sequence of DIQMTQSPASLSVSVGETVTITCRVSENIYSNLAWYQQKQGKSPQLLVYA ATNLADGVSSRFSGSVSGTQYSLKINSLQSEDFGSYYCQHFWGTLTFGAG TKLELK or a sequence at least 90% identical thereto;
e. a V_{H} according to the amino acid sequence of EVOLVESGGGLVKPGGSLKLSCAASGFTFSDYYIYWVRQTPEKRLEWVA TISDGGSYTYYPDSVKGRFTISRDNAKNNLYLQMSSLKSEDTAMYYCARG YYYGLFTYWGQGTLVTVSA or a sequence at least 90% identical thereto, and a V_{L} according to the amino acid sequence of DIVMTQSHKFMSTSVGDRVSITCKASQDVSTGVAWYROKPGRSPKLLIY WASTRHTGVPDRFTGSGSGTDYILTISSVQAEDLALYYCQQHYSTPYTFG GGTKLEIK or a sequence at least 90% identical thereto;
f. a V_{H} according to the amino acid sequence of QVQLKQSGPSLVQPSQSLSITCTVSGFSLTTYGVHWVRQSPGKGLEWLGV IWRGGSTDYNAGFMSRLSITKDNSKSQVFFKMNSLQADDTAIYYCAKNS YYYGGNYYFDYWGQGTTLTVSS or a sequence at least 90% identical thereto, and a V_{L} according to the amino acid sequence of DVVMTQTPFSLPVSLGDQASISCRSSQSLVHSNGNTHLHWYLQKPGQSPK LLIYKVSNRFSGVPDRFSGSGSGTDFTLKISRVEAEDLGVYFCSQSTHVPPT FGGGTKLEIK or a sequence at least 90% identical thereto;
g. a V_{H} according to the amino acid sequence of QVQLKQSGPSLVQPSQSLSINCTVSGFSLTTYGVHWLRQSPGKGLEWLGV IWRGGSTDYNAAFMSRLSITKDNSKSQVFFKMNSLQADDTAIYYCAKPY YVYGAMDYWGQGTSVTVSS or a sequence at least 90% identical thereto, and a V_{L} according to the amino acid sequence of DVLMTQTPLSLPVSLGDQASISCRSSESIVHSNGNTYLEWFLQKPGQSPKL LIYKVSNRFSGVPDRFSGSGSGTDFTLKISRVEAEDLGVYYCFQGSHVPLT FGAGTKLEIK or a sequence at least 90% identical thereto;
h. a V_{H} according to the amino acid sequence of QVQLQESGPGLVKPSETLSLTCAVSGGSLSSDYWTWIRQPAGKGLEWIGR IFATGTTNYNPSLKSRVTMSIDTSKNQFSLKLNSVTAADTAVYYCARDDW YFDLWGRGTLVTVSS or a sequence at least 90% identical thereto, and a VL according to the amino acid sequence of EIVMTQSPATLSVSPGERATLSCRASQSVSNNLAWYQERPGQAPRLLIYG ASTRTTGIPARFSGSGSGTEFILTISSLQPEDFAIYYCQQYNEWPWTFGQGT KVEIK or a sequence at least 90% identical thereto;
i. a V_{H} according to the amino acid sequence of QVQLQESGPGLVKPSETLSLTCTVSGGSISSDYWSWIRQPAGKRLEWIGRI FTSGSTNYNPSLKSRVTMSVDTSKNQFSLKLNSLTAADTAVYYCARDDW YFDLWGRGTLVTVSS or a sequence at least 90% identical thereto, and a VL according to the amino acid sequence of EIVMTQSPATLSVSPGERATLSCRASQSVSNNLAWYQQKPGQAPRFLIYG ASTRATGMPARFSGSGSGTEFTLTISSLQSEDFAVYFCQQYNEWPWTFGQ GTKVEIK or a sequence at least 90% identical thereto;
j. a V_{H} according to the amino acid sequence of QVQLQESGPGLVKPSETLSLTCTVSGGSISRDYWSWVRQPAGKGLEWIGR IFTNGVTNYNPSLKSRVTMSVDTSKTQFSLKLSSVTAADTAVYFCARDDW YLDLWGRGTLVTVSS or a sequence at least 90% identical thereto, and a VL according to the amino acid sequence of EIVMTQSPATLSVSPGERATLSCRASQSVSNNLAWYQQTPGQAPRFLIYG ASTGATGIPARFSGSGSGTEFTLTITSLQSEDFAVYFCQQYNDWPWTFGQG TKVEIK or a sequence at least 90% identical thereto;
k. a V_{H} according to the amino acid sequence of QVQMKQSGPGLVQPSQSLSITCTVSGFSLTTYGVHWVRQSPGKGLEWLG VIWSGGSTYYNAAFISRLSISKDNSKSQVIFKMNSLQVDDTAIYYCARWD GYSFYYGMDYWGQGTSVTVSS or a sequence at least 90% identical thereto, and a V_{L} according to the amino acid sequence of QAVVTQESALTTSPGETVTLTCRSSTGAVTTSNYANWVQEKPDHLFTGLI GSTNNRAPGVPARFSGSLIGDKAALIITGAQTEDEARYFCALWYSNHLVF GGGTKLTV_{L} or a sequence at least 90% identical thereto; or
l. a V_{H} according to the amino acid sequence of QVQLKQSGPGLVQPSQSLSITCTVSGFSLTSYGVHWVRQSPGKGLEWLGV IWSGGSTDYNAAFISRLSISKDNSKSQVFFKMNSLQADDTAIYYCARWDG DSFYYAMDYWGQGTSVTVSS or a sequence at least 90% identical thereto, and a V_{L} according to the amino acid sequence of QAVVIQESALTTSPGETVTLTCRSSTGAVTTSNYASWVQEKPDHLFIGLIG GTNNRAPGVPARFSGSLIGDKAALTITGAQTEDEAIYFCALWYSNHLVFG GGTKLTV_{L} or a sequence at least 90% identical thereto.

13. The monoclonal antibody of claim 1, wherein the antibody is linked to a therapeutic agent.

14. The monoclonal antibody of claim 13, wherein the therapeutic agent is a toxin, a radiolabel, a siRNA, a small molecule, or a cytokine.

15. The monoclonal antibody of claim 1, wherein the antibody is a single chain variable fragment (scFV).

16. A nucleic acid encoding the antibody according to any one of claims 1-15.

17. A nucleic acid encoding a monoclonal antibody, wherein the monoclonal antibody comprises a V_{H} amino acid sequence as indicated below, or a sequence at least 90% identical thereto:
a.
EVQLVESGGGLVQPGGSLRLSCAASGFTFSDYYMYWVRQAPGKGLEWVS
TISDGGSYTYYPDSVKGRFTISRDNAKNNLYLQMNSLRAEDTAVYYCAR
GYYYGLFTYWGQGTLVTVSS;
b.
EVQLVESGGGLVKPGGSLKLSCAASGFTFSDYYMYWVRQTPEKRLEWVA
TISDGGSYTYYPDSVKGRFTISRDNAKNNLYLQMSSLKSEDTAMFYCAR
GYYYGLFTYWGQGTLVTVSA;
c.
EVQLQQSGPDLVKPGASVRMSCKASGYTFTNYVMHWVKQRPGQGLEWIG
YISPYNDGTKYNEKFKGKATLTSDKSSSTAYMELNSLTSEDSAVYYCAG
GDYSYAMDYWGQGTSVTVSS;
d.
EVQLQQSGPDLVKPGASVKMSCKASGYTFTHYVMHWVKQKPGQGLEWIG
YISPYNDGIKYNEKFKGKATLTSDKSSSTAYMELSSLTSEDSAVYYCAG
GDYSYAMDYWGQGTSVTVSS;
e.
EVQLVESGGGLVKPGGSLKLSCAASGFTFSDYYIYWVRQTPEKRLEWVA
TISDGGSYTYYPDSVKGRFTISRDNAKNNLYLQMSSLKSEDTAMYYCAR
GYYYGLFTYWGQGTLVTVSA;
f.
QVQLKQSGPSLVQPSQSLSITCTVSGFSLTTYGVHWVRQSPGKGLEWLG
VIWRGGSTDYNAGFMSRLSITKDNSKSQVFFKMNSLQADDTAIYYCAKN
SYYYGGNYYFDYWGQGTTLTVSS;
g.
QVQLKQSGPSLVQPSQSLSINCTVSGFSLTTYGVHWLRQSPGKGLEWLG
VIWRGGSTDYNAAFMSRLSITKDNSKSQVFFKMNSLQADDTAIYYCAKP
YYVYGAMDYWGQGTSVTVSS;
h.
QVQLQESGPGLVKPSETLSLTCAVSGGSLSSDYWTWIRQPAGKGLEWIG
RIFATGTTNYNPSLKSRVTMSIDTSKNQFSLKLNSVTAADTAVYYCARD
DWYFDLWGRGTLVTVSS;
i.
QVQLQESGPGLVKPSETLSLTCTVSGGSISSDYWSWIRQPAGKRLEWIG
RIFTSGSTNYNPSLKSRVTMSVDTSKNQFSLKLNSLTAADTAVYYCARD
DWYFDLWGRGTLVTVSS;
j.
QVQLQESGPGLVKPSETLSLTCTVSGGSISRDYWSWVRQPAGKGLEWIG
RIFTNGVTNYNPSLKSRVTMSVDTSKTQFSLKLSSVTAADTAVYFCARD
DWYLDLWGRGTLVTVSS;
k.
QVQMKQSGPGLVQPSQSLSITCTVSGFSLTTYGVHWVRQSPGKGLEWLG
VIWSGGSTYYNAAFISRLSISKDNSKSQVIFKMNSLQVDDTAIYYCARW
DGYSFYYGMDYWGQGTSVTVSS;
or
l.
QVQLKQSGPGLVQPSQSLSITCTVSGFSLTSYGVHWVRQSPGKGLEWLG
VIWSGGSTDYNAAFISRLSISKDNSKSQVFFKMNSLQADDTAIYYCARW
DGDSFYYAMDYWGQGTSVTVSS.

18. A nucleic acid encoding a monoclonal antibody, wherein the monoclonal antibody comprises a V_{L} amino acid sequence as indicated below, or a sequence at least 90% identical thereto:
a.
DIQMTQSPSSLSASVGDRVTITCRASQDVNTGLNWYQQKPGQAPKLLIY
WASSRQSGVPSRFSGSGSGTDFTLTISSLQPEDFATYYCQQHYSTPYTF
GQGTKLEIK;
b.
DIVMTQSHKFMSTSVGDRVSITCKASQDVNTGVAWYQQKPGQSPKLLIY
WASTRHTGVPDRFTGSGSGTDYILTISSVQAEDLALYYCQQHYSTPYTF
GGGTKLEIK;
c.
DIQMTQSPASLSVSVGETVTITCRVSENIYSNLAWYHQKQGKSPQLLVY
AATNLADGVPSRFSGSKSGTQYSLKISSLQSEDFGSYYCQHFWGTLTFG
AGTKLELK;
d.
DIQMTQSPASLSVSVGETVTITCRVSENIYSNLAWYQQKQGKSPQLLVY
AATNLADGVSSRFSGSVSGTQYSLKINSLQSEDFGSYYCQHFWGTLTFG
AGTKLELK;
e.
DIVMTQSHKFMSTSVGDRVSITCKASQDVSTGVAWYRQKPGRSPKLLIY
WASTRHTGVPDRFTGSGSGTDYILTISSVQAEDLALYYCQQHYSTPYTF
GGGTKLEIK;
f.
DVVMTQTPFSLPVSLGDQASISCRSSQSLVHSNGNTHLHWYLQKPGQSP
KLLIYKVSNRFSGVPDRFSGSGSGTDFTLKISRVEAEDLGVYFCSQSTH
VPPTFGGGTKLEIK;
g.
DVLMTQTPLSLPVSLGDQASISCRSSESIVHSNGNTYLEWFLQKPGQSP
KLLIYKVSNRFSGVPDRFSGSGSGTDFTLKISRVEAEDLGVYYCFQGSH
VPLTFGAGTKLEIK;
h.
EIVMTQSPATLSVSPGERATLSCRASQSVSNNLAWYQERPGQAPRLLIY
GASTRTTGIPARFSGSGSGTEFILTISSLQPEDFAIYYCQQYNEWPWTF
GQGTKVEIK;
i.
EIVMTQSPATLSVSPGERATLSCRASQSVSNNLAWYQQKPGQAPRFLIY
GASTRATGMPARFSGSGSGTEFTLTISSLQSEDFAVYFCQQYNEWPWTF
GQGTKVEIK;
j.
EIVMTQSPATLSVSPGERATLSCRASQSVSNNLAWYQQTPGQAPRFLIY
GASTGATGIPARFSGSGSGTEFTLTITSLQSEDFAVYFCQQYNDWPWTF
GQGTKVEIK;
k.
QAVVTQESALTTSPGETVTLTCRSSTGAVTTSNYANWVQEKPDHLFTGL
IGSTNNRAPGVPARFSGSLIGDKAALIITGAQTEDEARYFCALWYSNHL
VFGGGTKLTVL;
or
l.
QAVVIQESALTTSPGETVTLTCRSSTGAVTTSNYASWVQEKPDHLFIGL
IGGTNNRAPGVPARFSGSLIGDKAALTITGAQTEDEAIYFCALWYSNHL
VFGGGTKLTVL.

19. A nucleic acid encoding a monoclonal antibody, wherein the monoclonal antibody comprises:
a. a heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
b. a heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
c. a heavy chain with three CDRs comprising the amino acid sequences GYTFTNYV, ISPYNDGT, and AGGDYSYAMDY respectively, and/or a light chain with three CDRs comprising the amino acid sequences ENIYSN, AAT, and QHFWGTLT respectively;
d. a heavy chain with three CDRs comprising the amino acid sequences GYTFTHYV, ISPYNDGI, and AGGDYSYAMDY respectively, and/or a light chain with three CDRs comprising the amino acid sequences ENIYSN, AAT, and QHFWGTLT respectively;
e. a heavy chain with three CDRs comprising the amino acid sequences GFTFSDYY, ISDGGSYT, and ARGYYYGLFTY respectively, and/or a light chain with three CDRs comprising the amino acid sequences QDVSTG, WAS, and QQHYSTPYT respectively;
f. a heavy chain with three CDRs comprising the amino acid sequences GFSLTTYG, IWRGGST, and AKNSYYYGGNYYFDY respectively, and/or a light chain with three CDRs comprising the amino acid sequences QSLVHSNGNTH, KVS, and SQSTHVPPT respectively;
g. a heavy chain with three CDRs comprising the amino acid sequences GFSLTTYG, IWRGGST, and AKPYYVYGAMDY respectively, and/or a light chain with three CDRs comprising the amino acid sequences ESIVHSNGNTY, KVS, and FQGSHVPLT respectively;
h. a heavy chain with three CDRs comprising the amino acid sequences GGSLSSDY, IFATGTT, and ARDDWYFDL respectively, and/or a light chain with three CDRs comprising the amino acid sequences QSVSNN, GAS, and QQYNEWPWT respectively;
i. a heavy chain with three CDRs comprising the amino acid sequences GGSISSDY, IFTSGST, and ARDDWYFDL respectively, and/or a light chain with three CDRs comprising the amino acid sequences QSVSNN, GAS, and QQYNEWPWT respectively;
j. a heavy chain with three CDRs comprising the amino acid sequences GGSISRDY, IFTNGVT, and ARDDWYLDL respectively, and/or a light chain with three CDRs comprising the amino acid sequences QSVSNN, GAS, and QQYNDWPWT respectively;
k. a heavy chain with three CDRs comprising the amino acid sequences GFSLTTYG, IWSGGST, and ARWDGYSFYYGMDY respectively, and/or a light chain with three CDRs comprising the amino acid sequences TGAVTTSNY, STN, and ALWYSNHLV respectively; or
l. a heavy chain with three CDRs comprising the amino acid sequences GFSLTSYG, IWSGGST, and ARWDGDSFYYAMDY respectively, and/or a light chain with three CDRs comprising the amino acid sequences TGAVTTSNY, GTN, and ALWYSNHLV respectively.

20. A nucleic acid encoding a monoclonal antibody, wherein the monoclonal antibody comprises:
a. a V_{H} according to the amino acid sequence of EVOLVESGGGLVQPGGSLRLSCAASGFTFSDYYMYWVRQAPGKGLEWV STISDGGSYTYYPDSVKGRFTISRDNAKNNLYLQMNSLRAEDTAVYYCAR GYYYGLFTYWGQGTLVTVSS or a sequence at least 90% identical thereto, and a V_{L} according to the amino acid sequence of DIQMTQSPSSLSASVGDRVTITCRASQDVNTGLNWYQQKPGQAPKLLIYW ASSRQSGVPSRFSGSGSGTDFTLTISSLQPEDFATYYCQQHYSTPYTFGQGT KLEIK or a sequence at least 90% identical thereto;
b. a V_{H} according to the amino acid sequence of EVOLVESGGGLVKPGGSLKLSCAASGFTFSDYYMYWVRQTPEKRLE WVATISDGGSYTYYPDSVKGRFTISRDNAKNNLYLQMSSLKSEDTAM FYCARGYYYGLFTYWGQGTLVTVSA or a sequence at least 90% identical thereto, and a V_{L} according to the amino acid sequence of DIVMTQSHKFMSTSVGDRVSITCKASQDVNTGVAWYQQKPGQSPKLL IYWASTRHTGVPDRFTGSGSGTDYILTISSVQAEDLALYYCQQHYSTP YTFGGGTKLEIK or a sequence at least 90% identical thereto;
c. a V_{H} according to the amino acid sequence of EVQLQQSGPDLVKPGASVRMSCKASGYTFTNYVMHWVKQRPGQGLE WIGYISPYNDGTKYNEKFKGKATLTSDKSSSTAYMELNSLTSEDSAVY YCAGGDYSYAMDYWGQGTSVTVSS or a sequence at least 90% identical thereto, and a V_{L} according to the amino acid sequence of DIQMTQSPASLSVSVGETVTITCRVSENIYSNLAWYHQKQGKSPQLLV YAATNLADGVPSRFSGSKSGTQYSLKISSLQSEDFGSYYCQHFWGTLT FGAGTKLELK or a sequence at least 90% identical thereto;
d. a V_{H} according to the amino acid sequence of EVQLQQSGPDLVKPGASVKMSCKASGYTFTHYVMHWVKQKPGQGL EWIGYISPYNDGIKYNEKFKGKATLTSDKSSSTAYMELSSLTSEDSAVY YCAGGDYSYAMDYWGQGTSVTVSS or a sequence at least 90% identical thereto, and a V_{L} according to the amino acid sequence of DIQMTQSPASLSVSVGETVTITCRVSENIYSNLAWYQQKQGKSPQLLV YAATNLADGVSSRFSGSVSGTQYSLKINSLQSEDFGSYYCQHFWGTLT FGAGTKLELK or a sequence at least 90% identical thereto;
e. a V_{H} according to the amino acid sequence of EVOLVESGGGLVKPGGSLKLSCAASGFTFSDYYIYWVRQTPEKRLEW VATISDGGSYTYYPDSVKGRFTISRDNAKNNLYLQMSSLKSEDTAMY YCARGYYYGLFTYWGQGTLVTVSA or a sequence at least 90% identical thereto, and a V_{L} according to the amino acid sequence of DIVMTQSHKFMSTSVGDRVSITCKASQDVSTGVAWYRQKPGRSPKLLI YWASTRHTGVPDRFTGSGSGTDYILTISSVQAEDLALYYCQQHYSTPY TFGGGTKLEIK or a sequence at least 90% identical thereto;
f. a V_{H} according to the amino acid sequence of QVQLKQSGPSLVQPSQSLSITCTVSGFSLTTYGVHWVRQSPGKGLEWL GVIWRGGSTDYNAGFMSRLSITKDNSKSQVFFKMNSLQADDTAIYYC AKNSYYYGGNYYFDYWGQGTTLTVSS or a sequence at least 90% identical thereto, and a V_{L} according to the amino acid sequence of DVVMTQTPFSLPVSLGDQASISCRSSQSLVHSNGNTHLHWYLQKPGQS PKLLIYKVSNRFSGVPDRFSGSGSGTDFTLKISRVEAEDLGVYFCSQST HVPPTFGGGTKLEIK or a sequence at least 90% identical thereto;
g. a V_{H} according to the amino acid sequence of QVQLKQSGPSLVQPSQSLSINCTVSGFSLTTYGVHWLRQSPGKGLEWL GVIWRGGSTDYNAAFMSRLSITKDNSKSQVFFKMNSLQADDTAIYYC AKPYYVYGAMDYWGQGTSVTVSS or a sequence at least 90% identical thereto, and a V_{L} according to the amino acid sequence of DVLMTQTPLSLPVSLGDQASISCRSSESIVHSNGNTYLEWFLQKPGQSP KLLIYKVSNRFSGVPDRFSGSGSGTDFTLKISRVEAEDLGVYYCFQGSH VPLTFGAGTKLEIK or a sequence at least 90% identical thereto;
h. a V_{H} according to the amino acid sequence of QVQLQESGPGLVKPSETLSLTCAVSGGSLSSDYWTWIRQPAGKGLEWI GRIFATGTTNYNPSLKSRVTMSIDTSKNQFSLKLNSVTAADTAVYYCA RDDWYFDLWGRGTLVTVSS or a sequence at least 90% identical thereto, and a V_{L} according to the amino acid sequence of EIVMTQSPATLSVSPGERATLSCRASQSVSNNLAWYQERPGQAPRLLI YGASTRTTGIPARFSGSGSGTEFILTISSLQPEDFAIYYCQQYNEWPWTF GQGTKVEIK or a sequence at least 90% identical thereto;
i. a V_{H} according to the amino acid sequence of QVQLQESGPGLVKPSETLSLTCTVSGGSISSDYWSWIRQPAGKRLEWI GRIFTSGSTNYNPSLKSRVTMSVDTSKNQFSLKLNSLTAADTAVYYCA RDDWYFDLWGRGTLVTVSS or a sequence at least 90% identical thereto, and a V_{L} according to the amino acid sequence of EIVMTQSPATLSVSPGERATLSCRASQSVSNNLAWYQQKPGQAPRFLI YGASTRATGMPARFSGSGSGTEFTLTISSLQSEDFAVYFCQQYNEWPW TFGQGTKVEIK or a sequence at least 90% identical thereto;
j. a V_{H} according to the amino acid sequence of QVQLQESGPGLVKPSETLSLTCTVSGGSISRDYWSWVRQPAGKGLEWI GRIFTNGVTNYNPSLKSRVTMSVDTSKTQFSLKLSSVTAADTAVYFCA RDDWYLDLWGRGTLVTVSS or a sequence at least 90% identical thereto, and a V_{L} according to the amino acid sequence of EIVMTQSPATLSVSPGERATLSCRASQSVSNNLAWYQQTPGQAPRFLI YGASTGATGIPARFSGSGSGTEFTLTITSLQSEDFAVYFCQQYNDWPW TFGQGTKVEIK or a sequence at least 90% identical thereto;
k. a V_{H} according to the amino acid sequence of QVQMKQSGPGLVQPSQSLSITCTVSGFSLTTYGVHWVRQSPGKGLEW LGVIWSGGSTYYNAAFISRLSISKDNSKSQVIFKMNSLQVDDTAIYYCA RWDGYSFYYGMDYWGQGTSVTVSS or a sequence at least 90% identical thereto, and a V_{L} according to the amino acid sequence of QAVVTQESALTTSPGETVTLTCRSSTGAVTTSNYANWVQEKPDHLFT GLIGSTNNRAPGVPARFSGSLIGDKAALIITGAQTEDEARYFCALWYSN HLVFGGGTKLTV_{L} or a sequence at least 90% identical thereto; or
l. a V_{H} according to the amino acid sequence of QVQLKQSGPGLVQPSQSLSITCTVSGFSLTSYGVHWVRQSPGKGLEWL GVIWSGGSTDYNAAFISRLSISKDNSKSQVFFKMNSLQADDTAIYYCA RWDGDSFYYAMDYWGQGTSVTVSS or a sequence at least 90% identical thereto, and a V_{L} according to the amino acid sequence of QAVVIQESALTTSPGETVTLTCRSSTGAVTTSNYASWVQEKPDHLFIGL IGGTNNRAPGVPARFSGSLIGDKAALTITGAQTEDEAIYFCALWYSNHL VFGGGTKLTV_{L} or a sequence at least 90% identical thereto.

21. A vector comprising the nucleic acid according to any one of claims 16-20.

22. A cell comprising the nucleic acid according to any one of claims 16-20, or the vector of claim 21.

23. A cell producing the monoclonal antibody of any one of claims 1-15.

24. A kit comprising the antibody according to any one of claims 1-15; a syringe, needle, or applicator for administration of the at least one antibody to a subject; and instructions for use.

25. An isolated multi-specific antibody comprising the monoclonal antibody of claim 1 or a fragment thereof and at least one additional monoclonal antibody or fragment thereof.

26. The multi-specific antibody of claim 25, wherein the antibody is a bi-specific antibody.

27. The multi-specific antibody of claim 25, wherein the at least one additional antigen-binding fragment has specificity to a molecule on an immune cell.

28. The multispecific antibody of claim 27, wherein the molecule is selected from the group consisting of B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, and KIR.

29. The multispecific antibody of claim 25, wherein the monoclonal antibody or fragment thereof and the at least one additional antibody or fragment thereof is independently selected from a Fab fragment, a single-chain variable fragment (scFv), or a single-domain antibody.

30. The multispecific antibody of claim 25, wherein the antibody is an IL-10 receptor blocking antibody or an IL-10 blocking antibody.

31. A nucleic acid encoding the multispecific antibody according to any one of claims 25-30.

32. A vector comprising the nucleic acid of claim 31.

33. A cell comprising the nucleic acid of claim 31, or the vector of claim 32.

34. A cell producing the bispecific antibody according to any one of claims 25-30.

35. A kit comprising the bispecific antibody according to any one of claims 25-30; a syringe, needle, or applicator for administration of the at least one antibody to a subject; and instructions for use.

36. The bispecific antibody of claim 25, wherein the antibody is linked to a therapeutic agent.

37. The bispecific antibody of claim 36, wherein the therapeutic agent is a toxin, a radiolabel, a siRNA, a small molecule, or a cytokine.

38. A pharmaceutical composition comprising one or more compositions as described herein, and a pharmaceutically acceptable carrier or excipient.

39. The pharmaceutical composition of claim 38, further comprising at least one additional therapeutic agent.

40. The pharmaceutical composition of claim 39, wherein the therapeutic agent is a toxin, a radiolabel, a siRNA, a small molecule, or a cytokine.

41. A nanoparticle, wherein the nanoparticles comprise: (a) a CD40 agonist antibody, (b) an antibody specific for IL-10 or IL-10R, and (c) monophosphoryl lipid A (MPL).

42. The nanoparticle of claim 41, wherein the CD40 agonist antibody is on the surface of the nanoparticle.

43. The nanoparticle of claim 41, wherein MPL is inside the nanoparticle.

44. The nanoparticle of claim 41, wherein the CD40 agonist antibody is selected from the group consisting of FGK45, CP-870,984, CP-870,983, APX005M, dacetuzumab, and ChiLob 7/4.

45. The nanoparticle of claim 41, wherein the IL-10 antibody is an IL-10 blocking antibody or an IL-10 receptor blocking antibody.

46. The nanoparticle of claim 45, wherein the IL-10 or IL-10R antibody is selected from the group consisting of 49G08_HIL2, 49G08, 48C11, 49H07, 48C06, 53D07, 53H08, 42B02, 43C05, 43F05, 15A02, and 21B08.

47. An engineered cell comprising a chimeric antigen receptor, wherein the chimeric antigen receptor comprises an extracellular ligand binding domain that is specific for Interleukin-10 (IL-10) or IL-10R.

48. The engineered cell of claim 47, wherein the extracellular ligand binding domain comprises an antibody or fragment thereof.

49. The engineered cell of claim 48, wherein the antibody comprises:
a. a heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
b. a heavy chain with three CDRs comprising the amino acid sequences GFTFSDYY, ISDGGSYT, and ARGYYYGLFTY respectively, and/or a light chain with three CDRs comprising the amino acid sequences QDVNTG, WAS, and QQHYSTPYT respectively;
c. a heavy chain with three CDRs comprising the amino acid sequences GYTFTNYV, ISPYNDGT, and AGGDYSYAMDY respectively, and/or a light chain with three CDRs comprising the amino acid sequences ENIYSN, AAT, and QHFWGTLT respectively;
d. a heavy chain with three CDRs comprising the amino acid sequences GYTFTHYV, ISPYNDGI, and AGGDYSYAMDY respectively, and/or a light chain with three CDRs comprising the amino acid sequences ENIYSN, AAT, and QHFWGTLT respectively;
e. a heavy chain with three CDRs comprising the amino acid sequences GFTFSDYY, ISDGGSYT, and ARGYYYGLFTY respectively, and/or a light chain with three CDRs comprising the amino acid sequences QDVSTG, WAS, and QQHYSTPYT respectively;
f. a heavy chain with three CDRs comprising the amino acid sequences GFSLTTYG, IWRGGST, and AKNSYYYGGNYYFDY respectively, and/or a light chain with three CDRs comprising the amino acid sequences QSLVHSNGNTH, KVS, and SQSTHVPPT respectively;
g. a heavy chain with three CDRs comprising the amino acid sequences GFSLTTYG, IWRGGST, and AKPYYVYGAMDY respectively, and/or a light chain with three CDRs comprising the amino acid sequences ESIVHSNGNTY, KVS, and FQGSHVPLT respectively;
h. a heavy chain with three CDRs comprising the amino acid sequences GGSLSSDY, IFATGTT, and ARDDWYFDL respectively, and/or a light chain with three CDRs comprising the amino acid sequences QSVSNN, GAS, and QQYNEWPWT respectively;
i. a heavy chain with three CDRs comprising the amino acid sequences GGSISSDY, IFTSGST, and ARDDWYFDL respectively, and/or a light chain with three CDRs comprising the amino acid sequences QSVSNN, GAS, and QQYNEWPWT respectively;
j. a heavy chain with three CDRs comprising the amino acid sequences GGSISRDY, IFTNGVT, and ARDDWYLDL respectively, and/or a light chain with three CDRs comprising the amino acid sequences QSVSNN, GAS, and QQYNDWPWT respectively;
k. a heavy chain with three CDRs comprising the amino acid sequences GFSLTTYG, IWSGGST, and ARWDGYSFYYGMDY respectively, and/or a light chain with three CDRs comprising the amino acid sequences TGAVTTSNY, STN, and ALWYSNHLV respectively; and
l. a heavy chain with three CDRs comprising the amino acid sequences GFSLTSYG, IWSGGST, and ARWDGDSFYYAMDY respectively, and/or a light chain with three CDRs comprising the amino acid sequences TGAVTTSNY, GTN, and ALWYSNHLV respectively.

50. The engineered cell of claim 48, wherein the antibody comprises a V_{H} amino acid sequence as indicated below, or a sequence at least 90% identical thereto:
a.
EVQLVESGGGLVQPGGSLRLSCAASGFTFSDYYMYWVRQAPGKGLEWVS
TISDGGSYTYYPDSVKGRFTISRDNAKNNLYLQMNSLRAEDTAVYYCAR
GYYYGLFTYWGQGTLVTVSS;
b.
EVQLVESGGGLVKPGGSLKLSCAASGFTFSDYYMYWVRQTPEKRLEWVA
TISDGGSYTYYPDSVKGRFTISRDNAKNNLYLQMSSLKSEDTAMFYCAR
GYYYGLFTYWGQGTLVTVSA;
c.
EVQLQQSGPDLVKPGASVRMSCKASGYTFTNYVMHWVKQRPGQGLEWIG
YISPYNDGTKYNEKFKGKATLTSDKSSSTAYMELNSLTSEDSAVYYCAG
GDYSYAMDYWGQGTSVTVSS;
d.
EVQLQQSGPDLVKPGASVKMSCKASGYTFTHYVMHWVKQKPGQGLEWIG
YISPYNDGIKYNEKFKGKATLTSDKSSSTAYMELSSLTSEDSAVYYCAG
GDYSYAMDYWGQGTSVTVSS;
e.
EVQLVESGGGLVKPGGSLKLSCAASGFTFSDYYIYWVRQTPEKRLEWVA
TISDGGSYTYYPDSVKGRFTISRDNAKNNLYLQMSSLKSEDTAMYYCAR
GYYYGLFTYWGQGTLVTVSA;
f.
QVQLKQSGPSLVQPSQSLSITCTVSGFSLTTYGVHWVRQSPGKGLEWLG
VIWRGGSTDYNAGFMSRLSITKDNSKSQVFFKMNSLQADDTAIYYCAKN
SYYYGGNYYFDYWGQGTTLTVSS;
g.
QVQLKQSGPSLVQPSQSLSINCTVSGFSLTTYGVHWLRQSPGKGLEWLG
VIWRGGSTDYNAAFMSRLSITKDNSKSQVFFKMNSLQADDTAIYYCAKP
YYVYGAMDYWGQGTSVTVSS;
h.
QVQLQESGPGLVKPSETLSLTCAVSGGSLSSDYWTWIRQPAGKGLEWIG
RIFATGTTNYNPSLKSRVTMSIDTSKNQFSLKLNSVTAADTAVYYCARD
DWYFDLWGRGTLVTVSS;
i.
QVQLQESGPGLVKPSETLSLTCTVSGGSISSDYWSWIRQPAGKRLEWIG
RIFTSGSTNYNPSLKSRVTMSVDTSKNQFSLKLNSLTAADTAVYYCARD
DWYFDLWGRGTLVTVSS;
j.
QVQLQESGPGLVKPSETLSLTCTVSGGSISRDYWSWVRQPAGKGLEWIG
RIFTNGVTNYNPSLKSRVTMSVDTSKTQFSLKLSSVTAADTAVYFCARD
DWYLDLWGRGTLVTVSS;
k.
QVQMKQSGPGLVQPSQSLSITCTVSGFSLTTYGVHWVRQSPGKGLEWLG
VIWSGGSTYYNAAFISRLSISKDNSKSQVIFKMNSLQVDDTAIYYCARW
DGYSFYYGMDYWGQGTSVTVSS;
or
l.
QVQLKQSGPGLVQPSQSLSITCTVSGFSLTSYGVHWVRQSPGKGLEWLG
VIWSGGSTDYNAAFISRLSISKDNSKSQVFFKMNSLQADDTAIYYCARW
DGDSFYYAMDYWGQGTSVTVSS.

51. The engineered cell of claim 48, wherein the antibody comprises a V_{L} amino acid sequence as indicated below, or a sequence at least 90% identical thereto:
a.
DIQMTQSPSSLSASVGDRVTITCRASQDVNTGLNWYQQKPGQAPKLLIY
YWASSRQSGVPSRFSGSGSGTDFTLTISSLQPEDFATYYCQQHYSTPYT
FGQGTKLEIK;
b.
DIVMTQSHKFMSTSVGDRVSITCKASQDVNTGVAWYQQKPGQSPKLLIY
WASTRHTGVPDRFTGSGSGTDYILTISSVQAEDLALYYCQQHYSTPYTF
GGGTKLEIK;
c.
DIQMTQSPASLSVSVGETVTITCRVSENIYSNLAWYHQKQGKSPQLLVY
AATNLADGVPSRFSGSKSGTQYSLKISSLQSEDFGSYYCQHFWGTLTFG
AGTKLELK;
d.
DIQMTQSPASLSVSVGETVTITCRVSENIYSNLAWYQQKQGKSPQLLVY
AATNLADGVSSRFSGSVSGTQYSLKINSLQSEDFGSYYCQHFWGTLTFG
AGTKLELK;
e.
DIVMTQSHKFMSTSVGDRVSITCKASQDVSTGVAWYRQKPGRSPKLLIY
WASTRHTGVPDRFTGSGSGTDYILTISSVQAEDLALYYCQQHYSTPYTF
GGGTKLEIK;
f.
DVVMTQTPFSLPVSLGDQASISCRSSQSLVHSNGNTHLHWYLQKPGQSP
KLLIYKVSNRFSGVPDRFSGSGSGTDFTLKISRVEAEDLGVYFCSQSTH
VPPTFGGGTKLEIK;
g.
DVLMTQTPLSLPVSLGDQASISCRSSESIVHSNGNTYLEWFLQKPGQSP
KLLIYKVSNRFSGVPDRFSGSGSGTDFTLKISRVEAEDLGVYYCFQGSH
VPLTFGAGTKLEIK;
h.
EIVMTQSPATLSVSPGERATLSCRASQSVSNNLAWYQERPGQAPRLLIY
GASTRTTGIPARFSGSGSGTEFILTISSLQPEDFAIYYCQQYNEWPWTF
GQGTKVEIK;
i.
EIVMTQSPATLSVSPGERATLSCRASQSVSNNLAWYQQKPGQAPRFLIY
GASTRATGMPARFSGSGSGTEFTLTISSLQSEDFAVYFCQQYNEWPWTF
GQGTKVEIK;
j.
EIVMTQSPATLSVSPGERATLSCRASQSVSNNLAWYQQTPGQAPRFLIY
GASTGATGIPARFSGSGSGTEFTLTITSLQSEDFAVYFCQQYNDWPWTF
GQGTKVEIK;
k.
QAVVTQESALTTSPGETVTLTCRSSTGAVTTSNYANWVQEKPDHLFTGL
IGSTNNRAPGVPARFSGSLIGDKAALIITGAQTEDEARYFCALWYSNHL
VFGGGTKLTVL;
or
l.
QAVVIQESALTTSPGETVTLTCRSSTGAVTTSNYASWVQEKPDHLFIGL
IGGTNNRAPGVPARFSGSLIGDKAALTITGAQTEDEAIYFCALWYSNHL
VFGGGTKLTVL.

52. The engineered cell of claim 48, wherein the antibody comprises:
a. a V_{H} according to the amino acid sequence of EVOLVESGGGLVQPGGSLRLSCAASGFTFSDYYMYWVRQAPGKGLE WVSTISDGGSYTYYPDSVKGRFTISRDNAKNNLYLQMNSLRAEDTAV YYCARGYYYGLFTYWGQGTLVTVSS or a sequence at least 90% identical thereto, and a V_{L} according to the amino acid sequence of DIQMTQSPSSLSASVGDRVTITCRASQDVNTGLNWYQQKPGQAPKLLI YWASSRQSGVPSRFSGSGSGTDFTLTISSLQPEDFATYYCQQHYSTPYT FGQGTKLEIK or a sequence at least 90% identical thereto;
b. a V_{H} according to the amino acid sequence of EVOLVESGGGLVKPGGSLKLSCAASGFTFSDYYMYWVRQTPEKRLE WVATISDGGSYTYYPDSVKGRFTISRDNAKNNLYLQMSSLKSEDTAM FYCARGYYYGLFTYWGQGTLVTVSA or a sequence at least 90% identical thereto, and a V_{L} according to the amino acid sequence of DIVMTQSHKFMSTSVGDRVSITCKASQDVNTGVAWYQQKPGQSPKLL IYWASTRHTGVPDRFTGSGSGTDYILTISSVQAEDLALYYCQQHYSTP YTFGGGTKLEIK or a sequence at least 90% identical thereto;
c. a V_{H} according to the amino acid sequence of EVQLQQSGPDLVKPGASVRMSCKASGYTFTNYVMHWVKQRPGQGLE WIGYISPYNDGTKYNEKFKGKATLTSDKSSSTAYMELNSLTSEDSAVY YCAGGDYSYAMDYWGQGTSVTVSS or a sequence at least 90% identical thereto, and a V_{L} according to the amino acid sequence of DIQMTQSPASLSVSVGETVTITCRVSENIYSNLAWYHQKQGKSPQLLV YAATNLADGVPSRFSGSKSGTQYSLKISSLQSEDFGSYYCQHFWGTLT FGAGTKLELK or a sequence at least 90% identical thereto;
d. a V_{H} according to the amino acid sequence of EVQLQQSGPDLVKPGASVKMSCKASGYTFTHYVMHWVKQKPGQGL EWIGYISPYNDGIKYNEKFKGKATLTSDKSSSTAYMELSSLTSEDSAVY YCAGGDYSYAMDYWGQGTSVTVSS or a sequence at least 90% identical thereto, and a V_{L} according to the amino acid sequence of DIQMTQSPASLSVSVGETVTITCRVSENIYSNLAWYQQKQGKSPQLLV YAATNLADGVSSRFSGSVSGTQYSLKINSLQSEDFGSYYCQHFWGTLT FGAGTKLELK or a sequence at least 90% identical thereto;
e. a V_{H} according to the amino acid sequence of EVOLVESGGGLVKPGGSLKLSCAASGFTFSDYYIYWVRQTPEKRLEW VATISDGGSYTYYPDSVKGRFTISRDNAKNNLYLQMSSLKSEDTAMY YCARGYYYGLFTYWGQGTLVTVSA or a sequence at least 90% identical thereto, and a V_{L} according to the amino acid sequence of DIVMTQSHKFMSTSVGDRVSITCKASQDVSTGVAWYROKPGRSPKLLI YWASTRHTGVPDRFTGSGSGTDYILTISSVQAEDLALYYCQQHYSTPY TFGGGTKLEIK or a sequence at least 90% identical thereto;
f. a V_{H} according to the amino acid sequence of QVQLKQSGPSLVQPSQSLSITCTVSGFSLTTYGVHWVRQSPGKGLEWL GVIWRGGSTDYNAGFMSRLSITKDNSKSQVFFKMNSLQADDTAIYYC AKNSYYYGGNYYFDYWGQGTTLTVSS or a sequence at least 90% identical thereto, and a V_{L} according to the amino acid sequence of DVVMTQTPFSLPVSLGDQASISCRSSQSLVHSNGNTHLHWYLQKPGQS PKLLIYKVSNRFSGVPDRFSGSGSGTDFTLKISRVEAEDLGVYFCSQST HVPPTFGGGTKLEIK or a sequence at least 90% identical thereto;
g. a V_{H} according to the amino acid sequence of QVQLKQSGPSLVQPSQSLSINCTVSGFSLTTYGVHWLRQSPGKGLEWL GVIWRGGSTDYNAAFMSRLSITKDNSKSQVFFKMNSLQADDTAIYYC AKPYYVYGAMDYWGQGTSVTVSS or a sequence at least 90% identical thereto, and a V_{L} according to the amino acid sequence of DVLMTQTPLSLPVSLGDQASISCRSSESIVHSNGNTYLEWFLQKPGQSP KLLIYKVSNRFSGVPDRFSGSGSGTDFTLKISRVEAEDLGVYYCFQGSH VPLTFGAGTKLEIK or a sequence at least 90% identical thereto;
h. a V_{H} according to the amino acid sequence of QVQLQESGPGLVKPSETLSLTCAVSGGSLSSDYWTWIRQPAGKGLEWI GRIFATGTTNYNPSLKSRVTMSIDTSKNQFSLKLNSVTAADTAVYYCA RDDWYFDLWGRGTLVTVSS or a sequence at least 90% identical thereto, and a V_{L} according to the amino acid sequence of EIVMTQSPATLSVSPGERATLSCRASQSVSNNLAWYQERPGQAPRLLI YGASTRTTGIPARFSGSGSGTEFILTISSLQPEDFAIYYCQQYNEWPWTF GQGTKVEIK or a sequence at least 90% identical thereto;
i. a V_{H} according to the amino acid sequence of QVQLQESGPGLVKPSETLSLTCTVSGGSISSDYWSWIRQPAGKRLEWI GRIFTSGSTNYNPSLKSRVTMSVDTSKNQFSLKLNSLTAADTAVYYCA RDDWYFDLWGRGTLVTVSS or a sequence at least 90% identical thereto, and a V_{L} according to the amino acid sequence of EIVMTQSPATLSVSPGERATLSCRASQSVSNNLAWYQQKPGQAPRFLI YGASTRATGMPARFSGSGSGTEFTLTISSLQSEDFAVYFCQQYNEWPW TFGQGTKVEIK or a sequence at least 90% identical thereto;
j. a V_{H} according to the amino acid sequence of QVQLQESGPGLVKPSETLSLTCTVSGGSISRDYWSWVRQPAGKGLEWI GRIFTNGVTNYNPSLKSRVTMSVDTSKTQFSLKLSSVTAADTAVYFCA RDDWYLDLWGRGTLVTVSS or a sequence at least 90% identical thereto, and a V_{L} according to the amino acid sequence of EIVMTQSPATLSVSPGERATLSCRASQSVSNNLAWYQQTPGQAPRFLI YGASTGATGIPARFSGSGSGTEFTLTITSLQSEDFAVYFCQQYNDWPW TFGQGTKVEIK or a sequence at least 90% identical thereto;
k. a V_{H} according to the amino acid sequence of QVQMKQSGPGLVQPSQSLSITCTVSGFSLTTYGVHWVRQSPGKGLEW LGVIWSGGSTYYNAAFISRLSISKDNSKSQVIFKMNSLQVDDTAIYYCA RWDGYSFYYGMDYWGQGTSVTVSS or a sequence at least 90% identical thereto, and a V_{L} according to the amino acid sequence of QAVVTQESALTTSPGETVTLTCRSSTGAVTTSNYANWVQEKPDHLFT GLIGSTNNRAPGVPARFSGSLIGDKAALIITGAQTEDEARYFCALWYSN HLVFGGGTKLTV_{L} or a sequence at least 90% identical thereto; or
l. a V_{H} according to the amino acid sequence of QVQLKQSGPGLVQPSQSLSITCTVSGFSLTSYGVHWVRQSPGKGLEWL GVIWSGGSTDYNAAFISRLSISKDNSKSQVFFKMNSLQADDTAIYYCA RWDGDSFYYAMDYWGQGTSVTVSS or a sequence at least 90% identical thereto, and a V_{L} according to the amino acid sequence of QAVVIQESALTTSPGETVTLTCRSSTGAVTTSNYASWVQEKPDHLFIGL IGGTNNRAPGVPARFSGSLIGDKAALTITGAQTEDEAIYFCALWYSNHL VFGGGTKLTV_{L} or a sequence at least 90% identical thereto.

53. The engineered cell of claim 47, wherein the cell comprises a T cell, an NK cell, or an NKT cell.

54. The engineered cell of claim 53, wherein the T cell is CD4+, CD8+, CD3+ pan T cells, or any combination thereof.

55. A kit comprising the engineered cell according to any one of claims 47-54; a syringe, needle, or applicator for administration of the engineered cell to a subject; and instructions for use.

56. A method of detecting the presence of a cancer in a sample, the method comprising contacting the sample with the monoclonal antibody according to any one of claims 1-15 or the multi-specific antibody according to any one of claims 25-30; and detecting the presence or absence of an antibody-antigen complex, thereby detecting the presence of IL-10 in the sample.

57. The method of claim 56 wherein contacting comprises immunohistochemistry.

58. The method of claim 57, wherein immunohistochemistry comprises precipitation, immunofluorescence, western blot, or ELISA.

59. The method of claim 56, wherein the sample is whole blood, a blood component, a body fluid, a biopsy, a tissue, serum or one or more cells.

60. The method of claim 56, wherein the sample comprises a normal sample or a cancerous sample.

61. The method of claim 59, wherein the one or more cells comprise an in vitro culture.

62. The method of claim 59, wherein the one or more cells comprise IL-10-expressing cells.

63. The method of claim 56, wherein the sample is an in vitro sample.

64. The method of claim 56, further comprising the step of obtaining a sample from a subject.

65. The method of claim 56, wherein the cancer expresses IL-10.

66. The method of claim 65, wherein IL-10 comprises full-length IL-10.

67. The method of claim 56, wherein the cancer comprises a solid tumor.

68. The method of 67, wherein the solid tumor comprises a melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor.

69. A method of treating cancer in a subject, the method comprising
contacting a sample obtained from a subject with a monoclonal antibody according to any one of claims 1-15 or the multispecific antibody according to any one of claims 25-30;
detecting the presence or absence of an antibody-antigen complex, wherein the presence of an antibody-antigen complex indicates the presence of cancer in the subject; and
administering to the subject an anticancer agent, thereby treating cancer in the subject.

70. The method of claim 69, wherein contacting comprises immunohistochemistry.

71. The method of claim 70, wherein immunohistochemistry comprises immunoprecipitation, immunofluorescence, western blot, ELISA.

72. The method of claim 69, wherein the sample is whole blood, a blood component, a body fluid, a biopsy, a tissue, serum or one or more cells.

73. The method of claim 69, wherein the sample comprises a normal sample or a cancerous sample.

74. The method of claim 72, wherein the one or more cells comprise an in vitro culture.

75. The method of claim 72, wherein the one or more cells comprise IL-10-expressing cells.

76. The method of claim 69, wherein the sample is an in vitro sample.

77. The method of claim 69, further comprising the step of obtaining a sample from a subject.

78. The method of claim 69, wherein the cancer expresses IL-10.

79. The method of claim 78, wherein IL-10 comprises full-length IL-10.

80. The method of claim 69, wherein the cancer comprises a solid tumor.

81. The method of 80, wherein the solid tumor comprises a melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor.

82. A method of treating cancer in a subject, the method comprising administering to a subject in need thereof a monoclonal antibody according to any one of claims 1-15, a multispecific antibody according to any one of claims 25-30, a nanoparticle according to any one of claims 41-46, or an engineered cell according to any one of claims 47-54.

83. The method of claim 82, wherein the cancer expresses IL-10.

84. The method of claim 83, wherein IL-10 comprises full-length IL-10.

85. The method of claim 82, wherein the composition is administered locally, such as intratumorally.

86. The method of claim 82, wherein the cancer comprises a solid tumor.

87. The method of 86, wherein the solid tumor comprises a melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor.

88. The method of claim 82, further comprising administering to the subject an anti-cancer agent.

89. A method of decreasing metastasis in a subject, the method comprising administering to a subject in need thereof a monoclonal antibody according to any one of claims 1-15, a multispecific antibody according to any one of claims 25-30, a nanoparticle according to any one of claims 41-46, or an engineered cell according to any one of claims 47-54.

90. The method of claim 89, wherein the subject is afflicted with a cancer.

91. The method of claim 90, wherein the cancer expresses IL-10.

92. The method of claim 91, wherein IL-10 comprises full-length IL-10.

93. The method of claim 89, wherein the composition is administered locally, such as intratumorally.

94. The method of claim 90, wherein the cancer comprises a solid tumor.

95. The method of claim 94, wherein the solid tumor comprises a melanoma, a breast tumor, a lung tumor, a small cell lung cancer tumor, a prostate tumor, an ovarian tumor, a sarcoma, hepatocellular carcinoma, a rectal tumor, a pancreatic tumor, a liver tumor, a biliary tract tumor, or a colon tumor.
